(19) United States
(12) Patent Application Publication
(10) Pub. No.: US 2014/0038889 A1
(43) Pub. Date: Feb. 6, 2014

(54) Modulators Of The GPR119 Receptor And The Treatment Of Disorders Related Thereto

(71) Applicant: Robert M. Jones, San Diego, CA (US)

(72) Inventor: Robert M. Jones, San Diego, CA (US)

(73) Assignee: ARENA PHARMACEUTICALS, INC., San Diego, CA (US)

(21) Appl. No.: 14/113,085

(22) PCT Filed: Apr. 20, 2012
(86) PCT No.: PCT/US12/34415

§ 371 Date: Oct. 21, 2013

Related U.S. Application Data
(60) Provisional application No. 61/478,283, filed on Apr. 22, 2011.

(51) Int. Cl. C07D 401/14; A61K 45/06; A61K 31/506

(52) U.S. Cl. CPC C07D 401/14; A61K 31/506; A61K 45/06
USPC 514/6.5; 544/319; 514/269

(57) ABSTRACT

The present invention relates to the GPR119 agonist, 1-methylcyclopropyl 4-(5-fluoro-6-(2-methyl-6-(methylsulfonyl)pyridin-3-yloxy)pyrimidin-4-yloxy)piperidine-1-carboxylate (Compound 1): and pharmaceutically acceptable salts, solvates, and hydrates thereof, that are useful as single pharmaceutical agents or in combination with one or more additional pharmaceutical agents, such as, a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, in the treatment of for example, a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; type 2 diabetes; obesity; and complications related thereto.

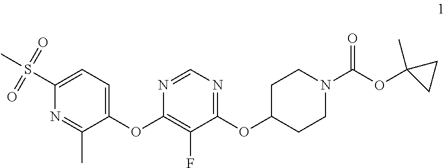 [SMILES: Cc1nc(S(C)(=O)=O)ccc1Oc1ncnc(OC2CCN(C(=O)OC3(C)CC3)CC2)c1F]

### FIELD OF THE INVENTION

The present invention relates to the GPR119 agonist, 1-methylcyclopropyl 4-(5-fluoro-6-(2-methyl-6-(methylsulfonyl)pyridin-3-yloxy)pyrimidin-4-yloxy)piperidine-1-carboxylate (Compound 1) and pharmaceutically acceptable salts, solvates, and hydrates thereof, that are useful as single pharmaceutical agents or in combination with one or more additional pharmaceutical agents, such as, a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, in the treatment of, for example, a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; type 2 diabetes; obesity; and complications related thereto.

### BACKGROUND OF THE INVENTION

A. Diabetes Mellitus

Diabetes mellitus is a serious disease afflicting over 100 million people worldwide. In the United States, there are more than 12 million diabetics, with 600,000 new cases diagnosed each year.

Diabetes mellitus is a diagnostic term for a group of disorders characterized by abnormal glucose homeostasis resulting in elevated blood sugar. There are many types of diabetes, but the two most common are type 1 (also referred to as insulin-dependent diabetes mellitus or IDDM) and type 2 (also referred to as non-insulin-dependent diabetes mellitus or NIDDM).

The etiology of the different types of diabetes is not the same; however, everyone with diabetes has two things in common: overproduction of glucose by the liver and little or no ability to move glucose out of the blood into the cells where it becomes the body's primary fuel.

People who do not have diabetes rely on insulin, a hormone made in the pancreas, to move glucose from the blood into the cells of the body. However, people who have diabetes either do not produce insulin or can not efficiently use the insulin they produce; therefore, they can not move glucose into their cells. Glucose accumulates in the blood creating a condition called hyperglycemia, and over time, can cause serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

About 5% to 10% of the people who have diabetes have IDDM. These individuals do not produce insulin and therefore must inject insulin to keep their blood glucose levels normal. IDDM is characterized by low or undetectable levels of endogenous insulin production caused by destruction of the insulin-producing beta-cells of the pancreas, the characteristic that most readily distinguishes IDDM from NIDDM. IDDM, once termed juvenile-onset diabetes, strikes young and older adults alike.

Approximately 90% to 95% of people with diabetes have type 2 (or NIDDM). NIDDM subjects produce insulin, but the cells in their bodies are insulin resistant: the cells do not respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production (Rotwein, R. et al., N. Engl. J. Med. 308, 65-71 (1983)). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious or even clinically unapparent, making diagnosis difficult.

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important contributors to the disease process (Rimoin, D. L., et. al. Emery and Rimoin's Principles and Practice of Medical Genetics 3^{rd} Ed. 1:1401-1402 (1996)).

Many people with NIDDM have sedentary lifestyles and are obese: they weigh approximately 20% more than the recommended weight for their height and build. Furthermore, obesity is characterized by hyperinsulinemia and insulin resistance, a feature shared with NIDDM, hypertension and atherosclerosis.

The patient with diabetes faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions (Perry, I. J., et al., BMJ 310, 560-564 (1995)).

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

B. Obesity

Obesity and diabetes are among the most common human health problems in industrialized societies. In industrialized countries a third of the population is at least 20% overweight. In the United States, the percentage of obese people has increased from 25% at the end of the 1970's, to 33% at the beginning the 1990's. Obesity is one of the most important risk factors for NIDDM. Definitions of obesity differ, but in general, a subject weighing at least 20% more than the recommended weight for his/her height and build is considered obese. The risk of developing NIDDM is tripled in subjects 30% overweight, and three-quarters with NIDDM are overweight.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increased insulin secretion balances insulin resistance and protects patients from hyperglycemia (Le Stunff, et al., Diabetes 43, 696-702 (1989)). However, after several decades, β cell function deteriorates and non-insulin-dependent diabetes develops in about 20% of the obese population (Pederson, P. Diab. Metab. Rev. 5, 505-509 (1989)) and (Brancati, F. L., et al., Arch. Intern. Med. 159, 957-963 (1999)). Given its high prevalence in modern societies, obesity has thus become the leading risk factor for NIDDM (Hill, J. O., et al., Science 280, 1371-1374 (1998)). However, the factors which predispose a fraction of patients to alteration of insulin secretion in response to fat accumulation remain unknown.

Whether someone is classified as overweight or obese can be determined by a number of different methods, such as, on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see table below). There are problems with this definition, such as, it does not take into account the proportion of body mass that is muscle in relation to fat (adipose tissue). To account for this, alternatively, obesity can be defined on the basis of body fat content: greater than 25% and 30% in males and females, respectively.

CLASSIFICATION OF WEIGHT
BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases associated with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight.

C. Atherosclerosis

Atherosclerosis is a complex disease characterized by inflammation, lipid accumulation, cell death and fibrosis. Atherosclerosis is characterized by cholesterol deposition and monocyte infiltration into the subendothelial space, resulting in foam cell formation. Thrombosis subsequent to atherosclerosis leads to myocardial infarction and stroke. Atherosclerosis is the leading cause of mortality in many countries, including the United States. (See, e.g., Ruggeri, Nat Med (2002) 8:1227-1234; Arehart et al., Circ Res, Circ. Res. (2008) 102:986-993.)

D. Osteoporosis

Osteoporosis is a disabling disease characterized by the loss of bone mass and microarchitectural deterioration of skeletal structure leading to compromised bone strength, which predisposes a patient to increased risk of fragility fractures. Osteoporosis affects more than 75 million people in Europe, Japan and the United States, and causes more than 2.3 million fractures in Europe and the United States alone. In the United States, osteoporosis affects at least 25% of all post-menopausal white women, and the proportion rises to 70% in women older than 80 years. One in three women older than 50 years will have an osteoporotic fracture that causes a considerable social and financial burden on society. The disease is not limited to women; older men also can be affected. By 2050, the worldwide incidence of hip fracture is projected to increase by 310% in men and 240% in women. The combined lifetime risk for hip, forearm, and vertebral fractures presenting clinically is around 40%, equivalent to the risk for cardiovascular disease. Osteoporotic fractures therefore cause substantial mortality, morbidity, and economic cost. With an ageing population, the number of osteoporotic fractures and their costs will at least double in the next 50 years unless effective preventive strategies are developed. (See, e.g., Atik et al., Clin Orthop Relat Res (2006) 443:19-24; Raisz, J Clin Invest (2005) 115:3318-3325; and World Health Organization Technical Report Series 921 (2003), Prevention and Management of Osteoporosis.)

E. Inflammatory Bowel Disease (IBD)

Inflammatory bowel disease (IBD) is the general name for diseases that cause inflammation in the intestines and includes, e.g. Crohn's disease (CD), ulcerative colitis (UC), ulcerative proctitis. U.S. medical costs of inflammatory bowel disease for 1990 have been estimated to be $1.4 to $1.8 billion. Lost productivity has been estimated to have added an additional $0.4 to $0.8 billion, making the estimated cost of inflammatory bowel disease $1.8 to $2.6 billion. (See, e.g., Pearson, Nursing Times (2004) 100:86-90; Hay et al., J Clin Gastroenterol (1992) 14:309-317; Keighley et al., Ailment Pharmacol Ther (2003) 18:66-70.)

Enteritis refers to inflammation of the intestine, especially the small intestine, a general condition that can have any of numerous different causes. Enterocolitis refers to inflammation of the small intestine and colon.

CD is an inflammatory process that can affect any portion of the digestive tract, but is most commonly seen in the last part of the small intestine otherwise called the (terminal) ileum and cecum. Altogether this area is also known as the ileocecal region. Other cases may affect one or more of: the colon only, the small bowel only (duodenum, jejunum and/or ileum), the anus, stomach or esophagus. In contrast with UC, CD usually does not affect the rectum, but frequently affects the anus instead. The inflammation extends deep into the lining of the affected organ. The inflammation can cause pain and can make the intestines empty frequently, resulting in diarrhea. CD may also be called enteritis. Granulomatous colitis is another name for CD that affects the colon. Ileitis is CD of the ileum which is the third part of the small intestine. Crohn's colitis is CD affecting part or all of the colon.

UC is an inflammatory disease of the large intestine, commonly called the colon. UC causes inflammation and ulceration of the inner lining of the colon and rectum. The inflammation of UC is usually most severe in the rectal area with severity diminishing (at a rate that varies from patient to patient) toward the cecum, where the large and small intestine join. Inflammation of the rectum is called proctitis. Inflammation of the sigmoid colon (located just above the rectum) is called sigmoiditis. Inflammation involving the entire colon is termed pancolitis. The inflammation causes the colon to empty frequently resulting in diarrhea. As the lining of the colon is destroyed ulcers form releasing mucus, pus and blood. Ulcerative proctitis is a form of UC that affects only the rectum.

F. GPR119

GPR119 is a G protein-coupled receptor (GPR119; e.g., human GPR119, GenBank® Accession No. AAP72125 and alleles thereof; e.g., mouse GPR119, GenBank® Accession No. AY288423 and alleles thereof) and is selectively expressed on pancreatic beta-cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo; see, e.g., International Applications WO04/065380 and WO04/076413, and EP1338651. In the literature, GPR119 has also been referred to as RUP3 (see, International Application WO00/31258) and as Glucose-Dependent Insulinotropic Receptor GDIR (see, Jones, et. al. Expert Opin. Ther. Patents (2009), 19(10): 1339-1359).

GPR119 agonists also stimulate the release of Glucose-dependent Insulinotropic Polypeptide (GIP), Glucagon-Like Peptide-1 (GLP-1), and at least one other L-cell peptide, Peptide YY (PYY) (Jones, et. al. Expert Opin. Ther. Patents (2009), 19(10): 1339-1359); for specific references related to GPR119 agonists and the release of:

GIP, see Shah, Current Opinion in Drug Discovery & Development, (2009) 12:519-532; Jones, et al., Ann. Rep. Med. Chem., (2009) 44:149-170; WO2007/120689; and WO2007/120702;

GLP-1, see Shah, Current Opinion in Drug Discovery & Development, (2009) 12:519-532; Jones, et al., Ann. Rep. Med. Chem., (2009) 44:149-170; Schwartz et. al., Cell Metabolism, 2010, 11:445-447; and WO2006/076231; and

PYY, see Schwartz et. al., Cell Metabolism, 2010, 11:445-447; and WO2009/126245.

As mentioned above, GPR119 agonists enhance incretin release and therefore can be used in treatment of disorders related to the incretins, such as, GIP, GLP-1, and PYY. However, a number of the incretins, such as, GIP and GLP-1, are substrates for the enzyme dipeptidyl peptidase-IV (DPP-IV). Jones and co-workers (Jones, et al., Ann. Rep. Med. Chem., (2009) 44:149-170) have demonstrated that a combined administration of a GPR119 agonist, (2-Fluoro-4-methanesulfonyl-phenyl)-{6-[4-(3-isopropyl-[1,2,4]oxadiazol-5-yl)-piperidin-1-yl]-5-nitro-pyrimidin-4-yl}-amine (see, compound B111 in WO2004/065380), and a DPP-IV inhibitor acutely increased plasma GLP-1 levels and improved glucose tolerance to a significantly greater degree than either agent alone.

G. Glucose-dependent Insulinotropic Polypeptide (GIP) Glucose-dependent insulinotropic polypeptide (GIP, also known as gastric inhibitory polypeptide) is a peptide incretin hormone of 42 amino acids that is released from duodenal endocrine K cells after meal ingestion. The amount of GIP released is largely dependent on the amount of glucose consumed. GIP has been shown to stimulate glucose-dependent insulin secretion in pancreatic beta-cells. GIP mediates its actions through a specific G protein-coupled receptor, namely GIPR.

As GIP contains an alanine at position 2, it is an excellent substrate for DPP-IV, an enzyme regulating the degradation of GIP. Full-length GIP(1-42) is rapidly converted to bioinactive GIP(3-42) within minutes of secretion from endocrine K cells. Inhibition of DPP-IV has been shown to augment GIP bioactivity. (See, e.g., Drucker, Cell Metab (2006) 3:153-165; McIntosh et al., Regul Pept (2005) 128:159-165; Deacon, Regul Pept (2005) 128:117-124; and Ahren et al., Endocrinology (2005) 146:2055-2059.). Analysis of full length bioactive GIP, for example in blood, can be carried out using N-terminal-specific assays (see, e.g., Deacon et al., J Clin Endocrinol Metab (2000) 85:3575-3581).

Recently, GIP has been shown to promote bone formation. GIP has been shown to activate osteoblastic receptors, resulting in increases in collagen type I synthesis and alkaline phosphatase activity, both associated with bone formation. GIP has been shown to inhibit osteoclast activity and differentiation in vitro. GIP administration has been shown to prevent the bone loss due to ovariectomy. GIP receptor (GIPR) knockout mice evidence a decreased bone size, lower bone mass, altered bone microarchitecture and biochemical properties, and altered parameters for bone turnover, especially in bone formation. (See, e.g., Thong et al., Am J Physiol Endocrinol Metab (2007) 292:E543-E548; Bollag et al., Endocrinology (2000) 141:1228-1235; Bollag et al., Mol Cell Endocrinol (2001) 177:35-41; Xie et al., Bone (2005) 37:759-769; and Tsukiyama et al., Mol Endocrinol (2006) 20:1644-1651.)

The usefulness of GIP for maintaining or increasing bone density or formation has been acknowledged by the United States Patent and Trademark Office by issuance of U.S. Pat. No. 6,410,508 for the treatment of reduced bone mineralization by administration of GIP peptide. However, current GIP peptide agonists suffer from a lack of oral bioavailability, negatively impacting patient compliance. An attractive alternative approach is to develop an orally active composition for increasing an endogenous level of GIP activity.

H. Glucagon-Like Peptide-1 (GLP-1)

Glucagon-like peptide-1 (GLP-1) is an incretin hormone derived from the posttranslational modification of proglucagon and secreted by gut endocrine cells. GLP-1 mediates its actions through a specific G protein-coupled receptor (GPCR), namely GLP-1R. GLP-1 is best characterized as a hormone that regulates glucose homeostasis. GLP-1 has been shown to stimulate glucose-dependent insulin secretion and to increase pancreatic beta cell mass. GLP-1 has also been shown to reduce the rate of gastric emptying and to promote satiety. The efficacy of GLP-1 peptide agonists in controlling blood glucose in type 2 diabetics has been demonstrated in several clinical studies [see, e.g., Nauck et al., Drug News Perspect (2003) 16:413-422], as has its efficacy in reducing body mass [Zander et al., Lancet (2002) 359:824-830].

GLP-1 receptor agonists are additionally useful in protecting against myocardial infarction and against cognitive and neurodegenerative disorders. GLP-1 has been shown to be cardioprotective in a rat model of myocardial infarction [Bose et al., Diabetes (2005) 54:146-151], and GLP-1R has been shown in rodent models to be involved in learning and neuroprotection [During et al., Nat. Med. (2003) 9:1173-1179; and Greig et al., Ann N Y Acad Sci (2004) 1035:290-315].

Certain disorders such as type 2 diabetes are characterized by a deficiency in GLP-1 [see, e.g., Nauck et al., Diabetes (2004) 53 Suppl 3:S190-196].

Current GLP-1 peptide agonists suffer from a lack of oral bioavailability. Alternative routes of administration are necessary, which negatively impacts patient compliance. Efforts to develop orally bioavailable non-peptidergic, small-molecule agonists of GLP-1R have so far been unsuccessful [Mentlein, Expert Opin Investig Drugs (2005) 14:57-64]. An attractive alternative approach is to develop an orally active composition for increasing an endogenous level of GLP-1 in the blood.

I. Peptide YY (PYY)

Peptide YY (PYY) is a 36 amino acid peptide originally isolated in 1980 from porcine intestine (Tatemoto et al., Nature (1980) 285:417-418). PYY is secreted from enteroendocrine L-cells within both the large and small intestine. It has been shown that in rat and human gut concentrations of immunoreactive PYY are low in duodenum and jejunum, high in ileum and colon, and highest in rectum (Lundberg et al., PNAS USA (1982) 79:4471-4475; Adrian et al., Gastroenterol. (1985) 89:1070-1077; Ekblad et al., Peptides (2002) 23:251-261; Ueno et al., Regul Pept (2008) 145:12-16). PYY expression in rat has also been reported to extend to alpha-cells of the islets of Langerhans and to cells in the medulla oblongata (Ekblad et al., Peptides (2002) 23:251-261). PYY is released into the circulation as PYY₁₋₃₆ and PYY₃₋₃₆ (Eberlein et al., Peptides (1989) 10:797-803). PYY₃₋₃₆ is generated from PYY₁₋₃₆ by cleavage of the N-terminal Tyr and Pro residues by DPP-IV. PYY₃₋₃₆ is the predominant form of PYY in human postprandial plasma (Grandt et al., Regul. Pept. (1994) 51:151-159). PYY₁₋₃₆ and PYY₃₋₃₆ have been reported to have comparable agonist activity at NPY Y2 receptor (Y2R), a G protein-coupled receptor (Parker et al., Br. J. Pharmacol. (2008) 153:420-431); however, PYY₃₋₃₆ has been reported to be a high-affinity Y2R selective agonist (Keire et al., Am. J. Physiol. Gastrointest. Liver Physiol. (2000) 279:G126-G131). PYY was subsequently reported to reduce high-fat food intake in rats after peripheral administration (Okada et al., Endocrinology Supplement (1993) 180) and to cause weight loss in mice after peripheral administration (Morley et al., Life Sciences (1987) 41:2157-2165).

Peripheral administration of PYY₃₋₃₆ has been reported to markedly reduce food intake and weight gain in rats, to decrease appetite and food intake in humans, and to decrease food intake in mice, but not in Y2R-null mice, which was said to suggest that the food intake effect requires the Y2R. In human studies, infusion of PYY₃₋₃₆ was found to significantly decrease appetite and reduce food intake by 33% over 24 hours. Infusion of PYY₃₋₃₆ to reach the normal postprandial circulatory concentrations of the peptide led to peak serum levels of PYY₃₋₃₆ within 15 minutes, followed by a rapid decline to basal levels within 30 minutes. It was reported that there was significant inhibition of food intake in the 12-hour period following the PYY₃₋₃₆ infusion, but essentially no effect on food intake in the 12-hour to 24-hour period. In a rat study, repeated administration of PYY₃₋₃₆ intraperitoneally (injections twice daily for 7 days) reduced cumulative food intake (Batterham et al., Nature (2002) 418:650-654; Renshaw et al., Current Drug Targets (2005) 6:171-179).

Peripheral administration of PYY₃₋₃₆ has been reported to reduce food intake, body weight gain and glycemic indices in diverse rodent models of metabolic diseases of both sexes (Pittner et al., Int. J. Obes. Relat. Metab. Disord. (2004) 28:963-971). It has been reported that blockade of Y2R with the specific antagonist BIIE-246 attenuates the effect of peripherally administered endogenous and exogenous PYY₃₋₃₆ for reducing food intake (Abbott et al., Brain Res (2005) 1043:139-144). It has been reported that peripheral administration of a novel long-acting selective Y2R polyethylene glycol-conjugated peptide agonist reduces food intake and improves glucose metabolism (glucose disposal, plasma insulin and plasma glucose) in rodents (Ortiz et al., JPET (2007) 323:692-700; Lamb et al., J. Med. Chem. (2007) 50:2264-2268). It has been reported that PYY ablation in mice leads to the development of hyperinsulinemia and obesity (Boey et al., Diabetologia (2006) 49:1360-1370). It has been reported that peripheral administration of a long-acting, potent and highly selective Y2R agonist inhibits food intake and promotes fat metabolism in mice (Balasubramaniam et al., Peptides (2007) 28:235-240).

There is evidence that agents which stimulate PYY synthesis in vivo can confer protection against diet-induced and genetic obesity and can improve glucose tolerance (Boey et al., Neuropeptides (2008) 42:19-30).

It has been reported that Y2R agonists such as PYY₁₋₃₆ and PYY₃₋₃₆ can confer protection against epileptic seizures, such as against kainate seizures (El Bahh et al., Eur. J. Neurosci. (2005) 22:1417-1430; Woldbye et al., Neurobiology of Disease (2005) 20:760-772).

It has been reported that Y2R agonists such as PYY₁₋₃₆ and PYY₃₋₃₆ act as proabsorbtive (or anti-secretory) hormones, increasing upon intravenous administration the absorption of both water and sodium in various parts of the bowel (Bilchik et al., Gastroenterol. (1993) 105:1441-1448; Liu et al., J. Surg. Res. (1995) 58:6-11; Nightingale et al., Gut (1996) 39:267-272; Liu et al., Am Surg (1996) 62:232-236; Balasubramaniam et al., J. Med. Chem. (2000) 43:3420-3427). It has been reported that Y2R agonists such as PYY analogues inhibit secretion and promote absorption and growth in the intestinal epithelium (Balasubramaniam et al., J. Med. Chem. (2000) 43:3420-3427). It has been reported that PYY promotes intestinal growth in normal rats (Gomez et al., Am. J. Physiol. (1995) 268:G71-G81). It has been reported that Y2R agonists such as PYY₁₋₃₆ and PYY₃₋₃₆ inhibit bowel motility and work to prevent diarrhea (EP1902730; also see Cox, Peptides (2007) 28:345-351).

It has been reported that Y2R agonists such as PYY₁₋₃₆ and PYY₃₋₃₆ can confer protection against inflammatory bowel disease such as UC and CD (WO 03/105763). It has been reported that PYY-deficient mice exhibit an osteopenic phenotype, i.e. that PYY can increase bone mass and/or can confer protection against loss of bone mass (e.g., decreases loss of bone mass) (Wortley et al., Gastroenterol. (2007) 133:1534-1543). It has been reported that PYY₃₋₃₆ can confer protection in rodent models of pancreatitis (Vona-Davis et al., Peptides (2007) 28:334-338).

It has been reported that angiogenesis is impaired in Y2R-deficient mice (Lee et al., Peptides (2003) 24:99-106), i.e. that agonists of Y2R such as PYY₁₋₃₆ and PYY₃₋₃₆ promote angiogenesis. It has been reported that wound healing is impaired in Y2R-deficient mice (Ekstrand et al., PNAS USA (2003) 100:6033-6038), i.e. that agonists of Y2R such as PYY₁₋₃₆ and PYY₃₋₃₆ promote wound healing. It has been reported that ischemic angiogenesis is impaired in Y2R-deficient mice (Lee et al., J. Clin. Invest. (2003) 111:1853-1862), i.e. that agonists of Y2R such as PYY₁₋₃₆ and PYY₃₋₃₆ promotes revascularization and restoration of function of ischemic tissue. It has been reported that agonists of Y2R such as PYY₁₋₃₆ and PYY₃₋₃₆ mediate increases in collateral-dependent blood flow in a rat model of peripheral arterial disease (Cruze et al., Peptides (2007) 28:269-280).

It has been reported that PYY and Y2R agonists such as PYY₃₋₃₆ can suppress tumor growth in the cases of, e.g., pancreatic cancer such as pancreatic ductal adenocarcinoma, breast cancer such as breast infiltrative ductal adenocarcinoma, colon cancer such as colon adenocarcinoma and Barrett's adenocarcinoma (Liu et al., Surgery (1995) 118:229-236; Liu et al., J. Surg. Res. (1995) 58:707-712; Grise et al., J. Surg. Res. (1999) 82:151-155; Tseng et al., Peptides (2002) 23:389-395; McFadden et al., Am. J. Surg. (2004) 188:516-519).

It has been reported that stimulation of Y2R such as by PYY₃₋₃₆ leads to an increase in plasma adiponectin (Ortiz et al., JPET (2007) 323:692-700). Adiponectin is an adipokine with potent anti-inflammatory properties (Ouchi et al., Clin Chim Acta (2007) 380:24-30; Tilg et al., Nat. Rev. Immunol. (2006) 6:772-783). Adiponectin exerts anti-atherogenic effects by targeting vascular endothelial cells and macrophages and insulin-sensitizing effects, predominantly in muscle and liver (Kubota et al., J. Biol. Chem. (2002) 277:25863-25866; Maeda et al., Nat. Med. (2002) 8:731-737). Low adiponectin levels have been reported to be associated with atherogenic lipoproteins in dyslipidemia (elevated triglycerides, small dense LDL cholesterol, low HDL cholesterol) (Marso et al., Diabetes Care (2008), 31(5), 989-994). Adiponectin has been implicated in high density lipoprotein (HDL) assembly (Oku et al., FEBS Letters (2007) 581:5029-5033). Adiponectin has been found to ameliorate the abnormalities of metabolic syndrome, including insulin resistance, hyperglycemia, and dyslipidemia, in a mouse model of obesity-linked metabolic syndrome associated with decreased adiponectin levels (Hara et al., Diabetes Care (2006) 29:1357-1362). Adiponectin has been reported to stimulate angiogenesis in response to tissue ischemia (Shibata et al., J. Biol. Chem. (2004) 279:28670-28674). Adiponectin has been reported to prevent cerebral ischemic injury through endothelial nitric oxide synthase-dependent mechanisms (Nishimura et al., Circulation (2008) 117:216-223).

Adiponectin has been reported to confer protection against myocardial ischemia-reperfusion injury (Shibata et al., Nat Med (2005) 11:1096-1103; Tao et al., Circulation (2007) 115:1408-1416). Adiponectin has been reported to confer protection against myocardial ischemia-reperfusion injury via AMP-activated protein kinase, Akt, and nitric oxide (Gonon et al., Cardiovasc Res. (2008) 78:116-122). Adiponectin has been reported to confer protection against the development of systolic dysfunction following myocardial infarction, through its abilities to suppress cardiac hypertrophy and interstitial fibrosis, and protect against myocyte and capillary loss (Shibata et al., J. Mol. Cell. Cardiol. (2007) 42:1065-1074). Adiponectin has been reported to confer protection against inflammatory lung disease; adiponectin-deficient mice exhibit an emphysema-like phenotype (Summer et al., Am J. Physiol. Lung Cell Mol. Physiol. (Mar. 7, 2008)). Adiponectin has been reported to confer protection against allergic airway inflammation and airway hyperresponsiveness such as may be associated with asthma (Shore et al., J. Allergy Clin. Immunol (2006) 118:389-395). Adiponectin has been suggested to confer protection against pulmonary arterial hypertension by virtue of its insulin-sensitizing effects (Hansmann et al., Circulation (2007) 115:1275-1284). Adiponectin has been reported to ameliorate obesity-related hypertension, with said amelioration of hypertension being associated in part with upregulated prostacyclin expression (Ohashi et al., Hypertension (2006) 47:1108-1116). Adiponectin has been reported to decrease tumor necrosis factor (TNF)-α-induced expression of the adhesion molecules VCAM-1, E-selectin and ICAM-1 in human aortic endothelial cells (HAECs) (Ouchi et al., Circulation (1999) 100:2473-2476) and to inhibit production of TNF-α in macrophages (Yokota et al., Blood (2000) 96:1723-1732). Adiponectin has been reported to confer protection against restenosis after vascular intervention (Matsuda et al., J Biol Chem (2002) 277:37487-37491). The central role of TNF-α in inflammation has been demonstrated by the ability of agents that block the action of TNF-α to treat a range of inflammatory conditions. TNF-α-mediated inflammatory conditions encompass rheumatoid arthritis, inflammatory bowel disease such as Crohn's disease, ankylosing spondylitis, psoriasis, ischemic brain injury, cardiac allograft rejection, asthma, and the like (Bradley, J Pathol (2008) 214:149-160). See, e.g., Yamamoto et al., Clinical Science (2002) 103:137-142; Behre, Scand J Clin Lab Invest (2007) 67:449-458; Guerre-Millo, Diabetes & Metabolism (2008) 34:12-18; Parker et al., Br. J. Pharmacol. (2008) 153:420-431.

### SUMMARY OF THE INVENTION

The present invention is drawn to 1-methylcyclopropyl 4-(5-fluoro-6-(2-methyl-6-(methylsulfonyl)pyridin-3-yloxy)pyrimidin-4-yloxy)piperidine-1-carboxylate (Compound 1) and pharmaceutically acceptable salts, solvates, and hydrates thereof, which bind to and modulate the activity of a GPCR, referred to herein as GPR119, and uses thereof.

One aspect of the present invention pertains to compounds selected from 1-methylcyclopropyl 4-(5-fluoro-6-(2-methyl-6-(methylsulfonyl)pyridin-3-yloxy)pyrimidin-4-yloxy)piperidine-1-carboxylate (Compound 1):

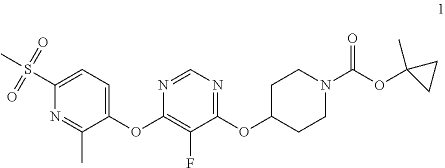 [SMILES: Cc1nc(S(C)(=O)=O)ccc1Oc1ncnc(OC2CCN(C(=O)OC3(C)CC3)CC2)c1F]

and pharmaceutically acceptable salts, solvates, and hydrates thereof.

One aspect of the present invention pertains to compositions comprising a compound of the present invention.

One aspect of the present invention pertains to compositions comprising a compound of the present invention and a pharmaceutically acceptable carrier.

One aspect of the present invention pertains to methods for preparing a composition comprising the step of admixing a compound of the present invention and a pharmaceutically acceptable carrier.

One aspect of the present invention pertains to compositions obtained by a method of the present invention.

One aspect of the present invention pertains to pharmaceutical products selected from: a pharmaceutical composition, a formulation, a dosage form, a combined preparation, a twin pack, and a kit; comprising a compound of the present invention.

One aspect of the present invention pertains to compositions comprising: a first pharmaceutical agent selected from a compound of the present invention; and a second pharmaceutical agent.

One aspect of the present invention pertains to methods for preparing a composition comprising: a first pharmaceutical agent selected from a compound of the present invention; and a second pharmaceutical agent.

One aspect of the present invention pertains to methods for preparing a composition comprising the step of admixing a first pharmaceutical agent selected from a compound of the present invention; and a second pharmaceutical agent.

One aspect of the present invention pertains to compositions comprising: a first pharmaceutical agent selected from a compound of the present invention; a second pharmaceutical agent; and a pharmaceutically acceptable carrier.

One aspect of the present invention pertains to methods for preparing a composition comprising the step of admixing a first pharmaceutical agent selected from a compound of the present invention; a second pharmaceutical agent; and a pharmaceutically acceptable carrier.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention; and a second pharmaceutical agent.

One aspect of the present invention pertains to methods for modulating the activity of a GPR119 receptor, comprising administering to an individual in need thereof, a therapeutically effective amount of: a compound, a composition, or a pharmaceutical product of the present invention.

One aspect of the present invention pertains to methods for modulating the activity of a GPR119 receptor, comprising prescribing to an individual in need thereof, a therapeutically effective amount of: a compound, a composition, or a pharmaceutical product of the present invention.

One aspect of the present invention pertains to methods for agonizing a GPR119 receptor, comprising administering to an individual in need thereof, a therapeutically effective amount of: a compound, a composition, or a pharmaceutical product of the present invention.

One aspect of the present invention pertains to methods for agonizing a GPR119 receptor, comprising prescribing to an individual in need thereof, a therapeutically effective amount of: a compound, a composition, or a pharmaceutical product of the present invention.

One aspect of the present invention pertains to methods for increasing the secretion of an incretin in an individual, comprising administering to the individual in need thereof, a therapeutically effective amount of: a compound, a composition, or a pharmaceutical product of the present invention.

One aspect of the present invention pertains to methods for increasing the secretion of an incretin in an individual, comprising prescribing to the individual in need thereof, a therapeutically effective amount of: a compound, a composition, or a pharmaceutical product of the present invention.

One aspect of the present invention pertains to methods for increasing a blood incretin level in an individual, comprising administering to the individual in need thereof, a therapeutically effective amount of: a compound, a composition, or a pharmaceutical product of the present invention.

One aspect of the present invention pertains to methods for increasing a blood incretin level in an individual, comprising prescribing to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual; comprising administering to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of: a compound, a composition, or a pharmaceutical product of the present invention.

One aspect of the present invention pertains to uses of a compound, a composition, or a pharmaceutical product of the present invention; in the manufacture of a medicament for modulating the activity of a GPR119 receptor in an individual.

One aspect of the present invention pertains to uses of a compound, a composition, or a pharmaceutical product of the present invention; in the manufacture of a medicament for agonizing a GPR119 receptor in an individual.

One aspect of the present invention pertains to uses of a compound, a composition, or a pharmaceutical product of the present invention; in the manufacture of a medicament for increasing the secretion of an incretin in an individual.

One aspect of the present invention pertains to uses of a compound, a composition, or a pharmaceutical product of the present invention; in the manufacture of a medicament for increasing a blood incretin level in an individual.

One aspect of the present invention pertains to uses of a compound, a composition, or a pharmaceutical product of the present invention; in the manufacture of a medicament for the treating a disorder in an individual, wherein the disorder is selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

One aspect of the present invention pertains to compounds, compositions, and pharmaceutical products of the present invention for use in a method of treatment of the human or animal body by therapy.

One aspect of the present invention pertains to compounds, compositions, and pharmaceutical products of the present invention for use in a method of modulating the activity of a GPR119 receptor in an individual.

One aspect of the present invention pertains to compounds, compositions, and pharmaceutical products of the present invention for use in a method of agonizing a GPR119 receptor in an individual.

One aspect of the present invention pertains to compounds, compositions, and pharmaceutical products of the present invention for use in a method of increasing the secretion of an incretin in an individual.

One aspect of the present invention pertains to compounds, compositions, and pharmaceutical products of the present invention for use in a method of increasing a blood incretin level in an individual.

One aspect of the present invention pertains to compounds, compositions, and pharmaceutical products of the present invention compounds, compositions, and pharmaceutical products of the present invention for use in a method of treatment of a disorder in an individual, wherein the disorder is selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

One aspect of the present invention pertains to pharmaceutical products selected from: a pharmaceutical composition, a formulation, a dosage form, a combined preparation, a twin pack, and a kit; comprising a compound of the present invention; for use in a method of treatment of the human or animal by therapy.

One aspect of the present invention pertains to pharmaceutical products selected from: a pharmaceutical composition, a formulation, a dosage form, a combined preparation, a twin pack, and a kit; comprising a compound of the present invention; for use in a method of modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for use in a method of agonizing a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for use in a method of increasing the secretion of an incretin in an individual.

One aspect of the present invention pertains to pharmaceutical products selected from: a pharmaceutical composition, a formulation, a dosage form, a combined preparation, a twin pack, and a kit; comprising a compound of the present invention; for use in a method of increasing a blood incretin level in an individual.

One aspect of the present invention pertains to pharmaceutical products selected from: a pharmaceutical composition, a formulation, a dosage form, a combined preparation, a twin pack, and a kit; comprising a compound of the present invention for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

One aspect of the present invention pertains to methods for modulating the activity of a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for modulating the activity of a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual; comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to uses of a first pharmaceutical agent selected from a compound of the present invention, in combination with a second pharmaceutical agent in the manufacture of a medicament for modulating the activity of a GPR119 receptor in an individual.

One aspect of the present invention pertains to uses of a first pharmaceutical agent in combination with a second pharmaceutical agent selected from a compound of the present invention, in the manufacture of a medicament for modulating the activity of a GPR119 receptor in an individual.

One aspect of the present invention pertains to uses of a first pharmaceutical agent selected from a compound of the present invention, in combination with a second pharmaceutical agent in the manufacture of a medicament for agonizing a GPR119 receptor in an individual.

One aspect of the present invention pertains to uses of a first pharmaceutical agent in combination with a second pharmaceutical agent selected from a compound of the present invention, in the manufacture of a medicament for agonizing a GPR119 receptor in an individual.

One aspect of the present invention pertains to uses of a first pharmaceutical agent selected from a compound of the present invention, in combination with a second pharmaceutical agent in the manufacture of a medicament for increasing the secretion of an incretin in an individual.

One aspect of the present invention pertains to uses of a first pharmaceutical agent in combination with a second pharmaceutical agent selected from a compound of the present invention, in the manufacture of a medicament for increasing the secretion of an incretin in an individual.

One aspect of the present invention pertains to uses of a first pharmaceutical agent selected from a compound of the present invention, in combination with a second pharmaceutical agent in the manufacture of a medicament for increasing a blood incretin level in an individual.

One aspect of the present invention pertains to uses of a first pharmaceutical agent in combination with a second pharmaceutical agent selected from a compound of the present invention, in the manufacture of a medicament for increasing a blood incretin level in an individual.

One aspect of the present invention pertains to uses of a first pharmaceutical agent selected from a compound of the present invention, in combination with a second pharmaceutical agent, in the manufacture of a medicament for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

One aspect of the present invention pertains to uses of a first pharmaceutical agent in combination with a second pharmaceutical agent selected from a compound of the present invention, in the manufacture of a medicament for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

One aspect of the present invention pertains to first pharmaceutical agents selected from a compound according of the present invention, for use in combination with a second pharmaceutical agent for use in a method of treatment of the human or animal body by therapy.

One aspect of the present invention pertains to first pharmaceutical agents selected from a compound according of the present invention, for use in combination with a second pharmaceutical agent for use in a method of modulating the activity of a GPR119 receptor in an individual.

One aspect of the present invention pertains to first pharmaceutical agents selected from a compound according of the present invention, for use in combination with a second pharmaceutical agent for use in a method of agonizing a GPR119 receptor in an individual.

One aspect of the present invention pertains to first pharmaceutical agents selected from a compound according of the present invention, for use in combination with a second pharmaceutical agent for use in a method of increasing the secretion of an incretin in an individual.

One aspect of the present invention pertains to first pharmaceutical agents selected from a compound according of the present invention, for use in combination with a second pharmaceutical agent for use in a method of increasing a blood incretin level in an individual.

One aspect of the present invention pertains to first pharmaceutical agents selected from a compound according of the present invention, for use in combination with a second pharmaceutical agent for use in a method of treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

One aspect of the present invention pertains to first pharmaceutical agents for use in combination with a second pharmaceutical agent selected from a compound of the present invention, for use in a method of treatment of the human or animal body by therapy.

One aspect of the present invention pertains to first pharmaceutical agents for use in combination with a second pharmaceutical agent selected from a compound of the present invention, for use in a method of modulating the activity of a GPR119 receptor in an individual.

One aspect of the present invention pertains to first pharmaceutical agents for use in combination with a second pharmaceutical agent selected from a compound of the present invention, for use in a method of agonizing a GPR119 receptor in an individual.

One aspect of the present invention pertains to first pharmaceutical agents for use in combination with a second pharmaceutical agent selected from a compound of the present invention, for use in a method of increasing the secretion of an incretin in an individual.

One aspect of the present invention pertains to first pharmaceutical agents for use in combination with a second pharmaceutical agent selected from a compound of the present invention, for use in a method of increasing a blood incretin level in an individual.

One aspect of the present invention pertains to first pharmaceutical agents for use in combination with a second pharmaceutical agent selected from a compound of the present invention, for use in a method of treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of the human or animal body by therapy.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of agonizing a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing the secretion of an incretin in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing a blood incretin level in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

One aspect of the present invention pertains to methods for preparing a pharmaceutical product of the present invention comprising: mixing the compound with a first pharmaceutically acceptable carrier to prepare a compound dosage form, mixing the second pharmaceutical agent with a second pharmaceutically acceptable carrier to prepare a second pharmaceutical agent dosage form, and providing the compound dosage form and the second pharmaceutical agent dosage form in a combined dosage form for simultaneous, separate, or sequential use.

One aspect of the present invention pertains to compositions comprising a compound of the present invention and a second pharmaceutical agent.

One aspect of the present invention pertains to methods for preparing a composition comprising the step of admixing a compound of the present invention and a second pharmaceutical agent.

One aspect of the present invention pertains to pharmaceutical products selected from: a pharmaceutical composition, a formulation, a dosage form, a combined preparation, a twin pack, and a kit; comprising a compound of the present invention and a second pharmaceutical agent.

One aspect of the present invention pertains to methods for increasing the secretion of an incretin in an individual, or for increasing a blood incretin level in an individual, comprising administering to said individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity; in an individual; comprising administering to said individual in need thereof, a therapeutically effective amount of: a compound, a composition, or a pharmaceutical product of the present invention.

One aspect of the present invention pertains to uses of a compound or a composition of the present invention in the manufacture of a medicament for increasing the secretion of an incretin in an individual, or for increasing a blood incretin level in an individual.

One aspect of the present invention pertains to uses of a compound or a composition of the present invention in the manufacture of a medicament for treating a disorder in an individual, wherein said disorder is selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity.

One aspect of the present invention pertains to compounds, compositions, and pharmaceutical products of the present invention for use in a method of treating the human or animal by therapy.

One aspect of the present invention pertains to compounds, compositions, and pharmaceutical products of the present invention for use in a method of increasing the secretion of an incretin in an individual or increasing a blood incretin level in an individual.

One aspect of the present invention pertains to compounds, compositions, and pharmaceutical products of the present invention for use in a method of treating a disorder in an individual, wherein said disorder is selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity.

One aspect of the present invention pertains to uses of a pharmaceutical agent in combination with a compound of the present invention, in the manufacture of a medicament for increasing the secretion of an incretin in an individual, or for increasing a blood incretin level in an individual.

One aspect of the present invention pertains to uses of a pharmaceutical agent in combination with a compound of the present invention, in the manufacture of a medicament for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity.

One aspect of the present invention pertains to pharmaceutical agents for use in combination with a compound or a composition of the present invention for use in a method of treating the human or animal by therapy.

One aspect of the present invention pertains to pharmaceutical agents for use in combination with a compound or a composition of the present invention for increasing the secretion of an incretin in an individual, or for increasing a blood incretin level in an individual.

One aspect of the present invention pertains to pharmaceutical agents for use in combination with a compound or a composition of the present invention for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity; in an individual.

These and other aspects of the invention disclosed herein will be set forth in greater detail as the patent disclosure proceeds.

### BRIEF DESCRIPTION OF THE DRAWINGS

FIG. 1 shows the effects of Compound 1 on glucose excursion in male 129SVE mice.

FIG. 2 shows the effects of Compound 1 on glucose excursion reduction in male 129SVE mice.

FIG. 3 shows the effects of Compound 1 on GIP release in male 129SVE mice.

FIG. 4 shows the effects of Compound 1 on glucose excursion in ZDF rats.

FIG. 5 shows the effects of Compound 1 on glucose excursion reduction in ZDF rats.

FIG. 6 shows the effects of Compound 1 on gastric emptying in SD rats.

### DETAILED DESCRIPTION OF THE INVENTION

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. Accordingly, all combinations of uses and medical indications described herein specifically embraced by the present invention just as if each and every subcombination of uses and medical indications was individually and explicitly recited herein.

The present disclosure includes all isotopes of atoms occurring in the present compounds, salts and crystalline forms thereof. Isotopes include those atoms having the same atomic number but different mass numbers. By way of general example, and without limitation, isotopes of hydrogen include ²H (deuterium) and ³H (tritium). Isotopes of carbon include ¹³C and ¹⁴C.

### DEFINITIONS

For clarity and consistency, the following definitions will be used throughout this patent document.

The term “agonist” as used herein refers to a moiety that interacts with and activates a G-protein-coupled receptor, for instance a GPR119-receptor, and can thereby initiate a physiological or pharmacological response characteristic of that receptor. For example, an agonist may activate an intracellular response upon binding to a receptor, or enhance GTP binding to a membrane.

The term “antagonist” as used herein refers to a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

The term “composition” refers to a compound, including but not limited to, salts, solvates, and hydrates of a compound of the present invention, in combination with at least one additional component.

The term “GPR119” as used herein includes the human amino acid sequences found in GeneBank accession number AY288416, and naturally-occurring allelic variants thereof, and mammalian orthologs thereof. A preferred human GPR119 for use in screening and testing of the compounds of the invention is provided in the nucleotide sequence of Seq. ID. No:1 and the corresponding amino acid sequence in Seq. ID. No:2 found in PCT Application No. WO2005/007647.

The term “in need of treatment” and the term “in need thereof” when referring to treatment are used interchangeably and refer to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” refers to any animal, including mammals, preferably mice, rats, other rodents, rabbits, dogs, cats, swine, cattle, sheep, horses, or primates, and most preferably humans.

The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. Preferably, the baseline intracellular response is inhibited in the presence of the inverse agonist by at least 30%, more preferably by at least 50% and most preferably by at least 75%, as compared with the baseline response in the absence of the inverse agonist.

The term “modulate or modulating” refers to an increase or decrease in the amount, quality, response or effect of a particular activity, function or molecule.

The term “partial agonist” refers to an agent (e.g., ligand, candidate compound) that by virtue of binding to a GPCR activates the GPCR so as to elicit an intracellular response mediated by the GPCR, albeit to a lesser extent or degree than does a full agonist.

The term “pharmaceutical composition” refers to a composition comprising at least one active ingredient, such as Compound 1 or a salt, solvate, or hydrate thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) preventing the disease, for example, preventing a disease, condition or disorder in an individual that may be predisposed to the disease, condition or disorder but does not yet experience or display the pathology or symptomatology of the disease;

(2) inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) ameliorating the disease, for example, ameliorating a disease, condition or disorder in an individual that is experiencing or displaying the pathology or symptomatology of the disease, condition or disorder (i.e., reversing the pathology and/or symptomatology).

The term “weight management” as used herein means controlling body weight and in the context of the present invention is directed toward weight loss and the maintenance of weight loss (also called weight maintenance herein). In addition to controlling body weight, weight management includes controlling parameters related to body weight, for example, BMI, percent body fat, and waist circumference. For example, weight management for an individual who is overweight or obese can refer to losing weight with the goal of keeping weight in a healthier range. Also, for example, weight management for an individual who is overweight or obese can include losing body fat or waist circumference with or without the loss of body weight.

The term “maintenance of weight loss” or “weight maintenance” as used herein includes preventing, reducing, or controlling weight gain after weight loss. It is well known that weight gain often occurs after weight loss. Weight loss can occur, for example, from dieting, exercising, illness, drug treatment, surgery, or any combination of these methods, but often an individual that has lost weight will regain some or all of the lost weight. Therefore, weight maintenance in an individual who has lost weight can include preventing weight gain after weight loss, reducing the amount of weight gained after weight loss, controlling weight gain after weight loss, or slowing the rate of weight gain after weight loss.

### Compounds

The novel compound 1-methylcyclopropyl 4-(5-fluoro-6-(2-methyl-6-(methylsulfonyl)pyridin-3-yloxy)pyrimidin-4-yloxy)piperidine-1-carboxylate (Compound 1) is a potent and selective agonist of GPR119. Compound 1 was prepared according to Scheme 1, below. (See Example 1).

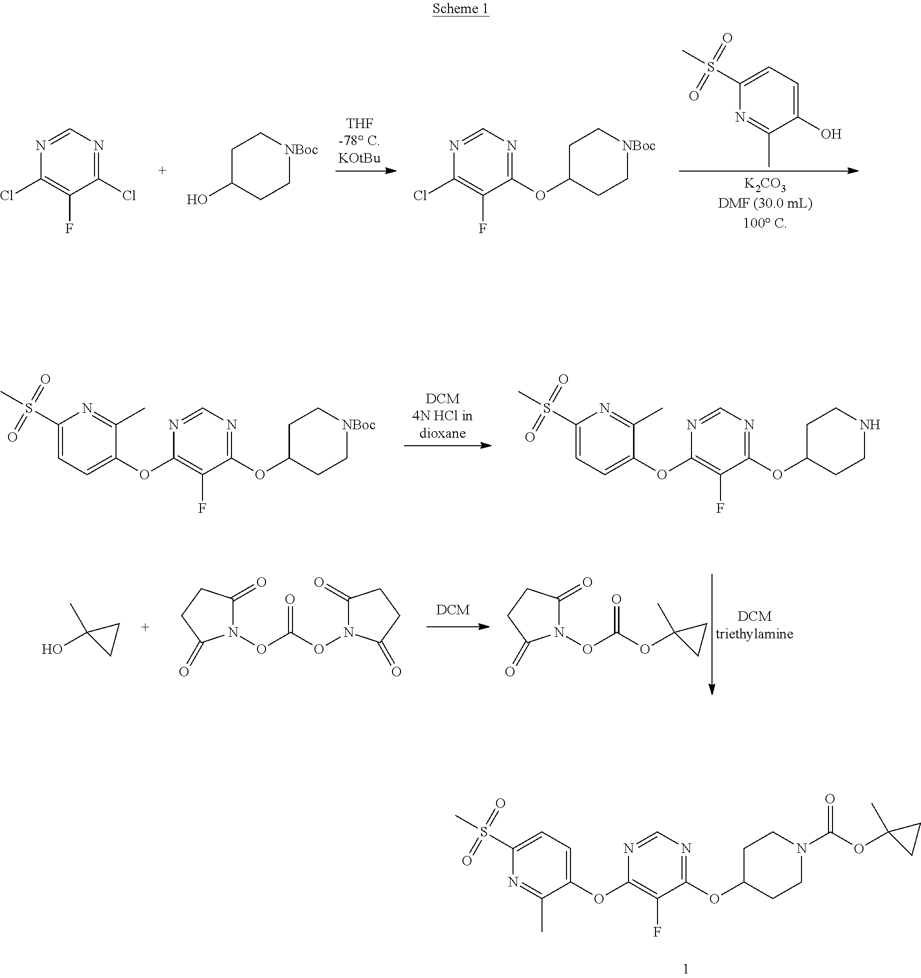

An oral glucose tolerance test (oGTT) demonstrated that Compound 1 significantly reduced plasma glucose levels in male 129SVE mice at a dose of 10 mg/kg. (See Example 2). Compound 1 also stimulated the release of GIP in male 129SVE mice. (See Example 3). Furthermore, using the HTRF® assay, Compound 1 was determined to be a full agonist of GPR119, with an EC₅₀ value of 14.9 nM. (See Example 4).

One aspect of the present invention pertains to compounds selected from 1-methylcyclopropyl 4-(5-fluoro-6-(2-methyl-6-(methylsulfonyl)pyridin-3-yloxy)pyrimidin-4-yloxy)piperidine-1-carboxylate (Compound 1):

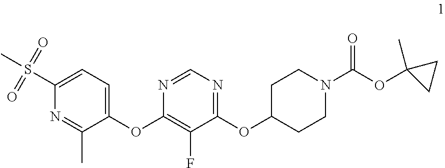 [SMILES: Cc1nc(S(C)(=O)=O)ccc1Oc1ncnc(OC2CCN(C(=O)OC3(C)CC3)CC2)c1F]

and pharmaceutically acceptable salts, solvates, and hydrates thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Some embodiments of the present invention include every combination of one or more uses or medical indications either specifically disclosed herein or specifically disclosed in any reference recited herein just as if each and every combination was individually and explicitly recited.

Some embodiments of the present invention include every combination of one or more pharmaceutical agents, such as a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, and the like, either specifically disclosed herein or specifically disclosed in any reference recited herein just as if each and every combination was individually and explicitly recited.

Some embodiments of the present invention include every combination of one or more embodiments pertaining to the compounds of the present invention in combination with every combination of one or more embodiments pertaining to the uses and medical indications of the present invention just as if each and every combination was individually and explicitly recited herein.

Some embodiments of the present invention include every combination of one or more embodiments pertaining to the compounds of the present invention in combination with every combination of one or more pharmaceutical agents, such as a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, and the like, either specifically disclosed herein or specifically disclosed in any reference recited herein just as if each and every combination was individually and explicitly recited.

Some embodiments of the present invention include every combination of one or more embodiments pertaining to the compounds of the present invention in combination with every combination of one or more embodiments pertaining to the uses and medical indications of the present invention just as if each and every combination was individually and explicitly recited herein, in combination with every combination of one or more pharmaceutical agents, such as a DPP-IV inhibitor, a biguanide, an alpha-glucosidase inhibitor, an insulin analogue, a sulfonylurea, an SGLT2 inhibitor, a meglitinide, a thiazolidinedione, or an anti-diabetic peptide analogue, and the like, either specifically disclosed herein or specifically disclosed in any reference recited herein just as if each and every combination was individually and explicitly recited.

One aspect of the present invention pertains to compounds, compositions, and pharmaceutical products of the present invention for use in a method of treatment of the human or animal body by therapy.

One aspect of the present invention pertains to compounds, compositions, and pharmaceutical products of the present invention for use in a method of modulating the activity of a GPR119 receptor in an individual.

One aspect of the present invention pertains to compounds, compositions, and pharmaceutical products of the present invention for use in a method of agonizing a GPR119 receptor in an individual.

One aspect of the present invention pertains to compounds, compositions, and pharmaceutical products of the present invention for use in a method of increasing the secretion of an incretin in an individual.

One aspect of the present invention pertains to compounds, compositions, and pharmaceutical products of the present invention for use in a method of increasing a blood incretin level in an individual.

One aspect of the present invention pertains to compounds, compositions, and pharmaceutical products of the present invention compounds, compositions, and pharmaceutical products of the present invention for use in a method of treatment of a disorder in an individual, wherein the disorder is selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

### Pharmaceutical Products, Methods, and Uses

One aspect of the present invention pertains to pharmaceutical products selected from: a pharmaceutical composition, a formulation, a dosage form, a combined preparation, a twin pack, and a kit; comprising a compound of the present invention.

One aspect of the present invention pertains to methods for preparing a composition comprising the step of admixing a compound of the present invention and a pharmaceutically acceptable carrier.

One aspect of the present invention pertains to methods for preparing a composition comprising: a first pharmaceutical agent selected from a compound of the present invention; and a second pharmaceutical agent.

One aspect of the present invention pertains to methods for preparing a composition comprising the step of admixing a first pharmaceutical agent selected from a compound of the present invention; a second pharmaceutical agent; and a pharmaceutically acceptable carrier.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention; and a second pharmaceutical agent.

One aspect of the present invention pertains to methods for modulating the activity of a GPR119 receptor, comprising administering to an individual in need thereof, a therapeutically effective amount of: a compound, a composition, or a pharmaceutical product of the present invention.

One aspect of the present invention pertains to methods for modulating the activity of a GPR119 receptor, comprising prescribing to an individual in need thereof, a therapeutically effective amount of: a compound, a composition, or a pharmaceutical product of the present invention.

One aspect of the present invention pertains to methods for agonizing a GPR119 receptor, comprising administering to an individual in need thereof, a therapeutically effective amount of: a compound, a composition, or a pharmaceutical product of the present invention.

One aspect of the present invention pertains to methods for agonizing a GPR119 receptor, comprising prescribing to an individual in need thereof, a therapeutically effective amount of: a compound, a composition, or a pharmaceutical product of the present invention.

One aspect of the present invention pertains to methods for increasing the secretion of an incretin in an individual, comprising administering to the individual in need thereof, a therapeutically effective amount of: a compound, a composition, or a pharmaceutical product of the present invention.

One aspect of the present invention pertains to methods for increasing the secretion of an incretin in an individual, comprising prescribing to the individual in need thereof, a therapeutically effective amount of: a compound, a composition, or a pharmaceutical product of the present invention.

One aspect of the present invention pertains to methods for increasing a blood incretin level in an individual, comprising administering to the individual in need thereof, a therapeutically effective amount of: a compound, a composition, or a pharmaceutical product of the present invention.

One aspect of the present invention pertains to methods for increasing a blood incretin level in an individual, comprising prescribing to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual; comprising administering to the individual in need thereof, a therapeutically effective amount of: a compound, a composition, or a pharmaceutical product of the present invention.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of: a compound, a composition, or a pharmaceutical product of the present invention.

One aspect of the present invention pertains to uses of a compound, a composition, or a pharmaceutical product of the present invention; in the manufacture of a medicament for modulating the activity of a GPR119 receptor in an individual.

One aspect of the present invention pertains to uses of a compound, a composition, or a pharmaceutical product of the present invention; in the manufacture of a medicament for agonizing a GPR119 receptor in an individual.

One aspect of the present invention pertains to uses of a compound, a composition, or a pharmaceutical product of the present invention; in the manufacture of a medicament for increasing the secretion of an incretin in an individual.

One aspect of the present invention pertains to uses of a compound, a composition, or a pharmaceutical product of the present invention; in the manufacture of a medicament for increasing a blood incretin level in an individual.

One aspect of the present invention pertains to uses of a compound, a composition, or a pharmaceutical product of the present invention; in the manufacture of a medicament for the treating a disorder in an individual, wherein the disorder is selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

One aspect of the present invention pertains to pharmaceutical products selected from: a pharmaceutical composition, a formulation, a dosage form, a combined preparation, a twin pack, and a kit; comprising a compound of the present invention; for use in a method of treatment of the human or animal by therapy.

One aspect of the present invention pertains to pharmaceutical products selected from: a pharmaceutical composition, a formulation, a dosage form, a combined preparation, a twin pack, and a kit; comprising a compound of the present invention; for use in a method of modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for use in a method of agonizing a GPR119 receptor in an individual.

One aspect of the present invention pertains to pharmaceutical products selected from: a pharmaceutical composition, a formulation, a dosage form, a combined preparation, a twin pack, and a kit; comprising a compound of the present invention; for use in a method of increasing the secretion of an incretin in an individual.

One aspect of the present invention pertains to pharmaceutical products selected from: a pharmaceutical composition, a formulation, a dosage form, a combined preparation, a twin pack, and a kit; comprising a compound of the present invention; for use in a method of increasing a blood incretin level in an individual.

One aspect of the present invention pertains to pharmaceutical products selected from: a pharmaceutical composition, a formulation, a dosage form, a combined preparation, a twin pack, and a kit; comprising a compound of the present invention for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

One aspect of the present invention pertains to methods for modulating the activity of a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising prescribing to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual, comprising prescribing to the individual in need thereof, a therapeutically effective amount of a first pharmaceutical agent selected from a compound of the present invention, in combination with a therapeutically effective amount of a second pharmaceutical agent.

One aspect of the present invention pertains to methods for modulating the activity of a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for agonizing a GPR119 receptor, comprising administering to an individual in need thereof, a therapeutically effective amount of a first pharmaceutical agent selected from a compound of the present invention, in combination with a therapeutically effective amount of a second pharmaceutical agent.

One aspect of the present invention pertains to methods for increasing the secretion of an incretin in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for increasing a blood incretin level in an individual, comprising administering to the individual in need thereof, a therapeutically effective amount of a first pharmaceutical agent selected from a compound of the present invention, in combination with a therapeutically effective amount of a second pharmaceutical agent.

One aspect of the present invention pertains to methods for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual; comprising administering to the individual in need thereof, a therapeutically effective amount of a first pharmaceutical agent selected from a compound of the present invention, in combination with a therapeutically effective amount of a second pharmaceutical agent.

In some embodiments, the first pharmaceutical agent and the second pharmaceutical agent are administered simultaneously, separately, or sequentially.

In some embodiments, the first pharmaceutical agent and the second pharmaceutical agent are administered simultaneously.

In some embodiments, the first pharmaceutical agent and the second pharmaceutical agent are administered separately.

In some embodiments, the first pharmaceutical agent and the second pharmaceutical agent are administered sequentially.

One aspect of the present invention pertains to uses of a first pharmaceutical agent selected from a compound of the present invention, in combination with a second pharmaceutical agent in the manufacture of a medicament for modulating the activity of a GPR119 receptor in an individual.

One aspect of the present invention pertains to uses of a first pharmaceutical agent in combination with a second pharmaceutical agent selected from a compound of the present invention, in the manufacture of a medicament for modulating the activity of a GPR119 receptor in an individual.

One aspect of the present invention pertains to uses of a first pharmaceutical agent selected from a compound of the present invention, in combination with a second pharmaceutical agent in the manufacture of a medicament for agonizing a GPR119 receptor in an individual.

One aspect of the present invention pertains to uses of a first pharmaceutical agent in combination with a second pharmaceutical agent selected from a compound of the present invention, in the manufacture of a medicament for agonizing a GPR119 receptor in an individual.

One aspect of the present invention pertains to uses of a first pharmaceutical agent selected from a compound of the present invention, in combination with a second pharmaceutical agent in the manufacture of a medicament for increasing the secretion of an incretin in an individual.

One aspect of the present invention pertains to uses of a first pharmaceutical agent in combination with a second pharmaceutical agent selected from a compound of the present invention, in the manufacture of a medicament for increasing the secretion of an incretin in an individual.

One aspect of the present invention pertains to uses of a first pharmaceutical agent selected from a compound of the present invention, in combination with a second pharmaceutical agent in the manufacture of a medicament for increasing a blood incretin level in an individual.

One aspect of the present invention pertains to uses of a first pharmaceutical agent in combination with a second pharmaceutical agent selected from a compound of the present invention, in the manufacture of a medicament for increasing a blood incretin level in an individual.

One aspect of the present invention pertains to uses of a first pharmaceutical agent selected from a compound of the present invention, in combination with a second pharmaceutical agent, in the manufacture of a medicament for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

One aspect of the present invention pertains to uses of a first pharmaceutical agent in combination with a second pharmaceutical agent selected from a compound of the present invention, in the manufacture of a medicament for the treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity.

In some embodiments, the treatment comprises administering the first pharmaceutical agent and the second pharmaceutical agent simultaneously, separately, or sequentially.

In some embodiments, the treatment comprises administering the first pharmaceutical agent and the second pharmaceutical agent simultaneously.

In some embodiments, the treatment comprises administering the first pharmaceutical agent and the second pharmaceutical agent separately.

In some embodiments, the treatment comprises administering the first pharmaceutical agent and the second pharmaceutical agent sequentially.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of the human or animal body by therapy.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of modulating the activity of a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of agonizing a GPR119 receptor in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing the secretion of an incretin in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of increasing a blood incretin level in an individual.

One aspect of the present invention pertains to pharmaceutical products selected from: a pharmaceutical composition, a formulation, a dosage form, a combined preparation, a twin pack, and a kit; comprising a first pharmaceutical agent selected from a compound of the present invention, and a second pharmaceutical agent; for use in a method of treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

In some embodiments, the method comprises administering the first pharmaceutical agent and the second pharmaceutical agent simultaneously, separately, or sequentially.

In some embodiments, the method comprises administering the first pharmaceutical agent and the second pharmaceutical agent simultaneously.

In some embodiments, the method comprises administering the first pharmaceutical agent and the second pharmaceutical agent separately.

In some embodiments, the method comprises administering the first pharmaceutical agent and the second pharmaceutical agent sequentially.

In some embodiments, the pharmaceutical product comprises a pharmaceutical composition.

In some embodiments, the pharmaceutical product comprises a formulation.

In some embodiments, the pharmaceutical product comprises a dosage form.

In some embodiments, the pharmaceutical product comprises a combined preparation,

In some embodiments, the pharmaceutical product comprises a twin pack.

In some embodiments, the pharmaceutical product comprises a kit.

One aspect of the present invention pertains to methods for preparing a pharmaceutical product of the present invention comprising: mixing the compound with a first pharmaceutically acceptable carrier to prepare a compound dosage form, mixing the second pharmaceutical agent with a second pharmaceutically acceptable carrier to prepare a second pharmaceutical agent dosage form, and providing the compound dosage form and the second pharmaceutical agent dosage form in a combined dosage form for simultaneous, separate, or sequential use.

In some embodiments, the first pharmaceutically acceptable carrier and the second pharmaceutically acceptable carrier are different pharmaceutically acceptable carriers.

In some embodiments, the different pharmaceutically acceptable carriers are suitable for administration by the same route or different routes.

In some embodiments, the first pharmaceutically acceptable carrier and the second pharmaceutically acceptable carrier are substantially the same pharmaceutically acceptable carriers.

In some embodiments, the substantially the same pharmaceutically acceptable carriers are suitable for administration by the same route.

In some embodiments, the substantially the same pharmaceutically acceptable carriers are suitable for oral administration.

In some embodiments, the incretin is GLP-1.

In some embodiments, the incretin is GIP.

In some embodiments, the incretin is PYY.

In some embodiments, the disorder is a GPR119-receptor-related disorder.

In some embodiments, the disorder is selected from: a condition ameliorated by increasing the secretion of an incretin, and a condition ameliorated by increasing a blood incretin level; and the incretin is GLP-1.

In some embodiments, the disorder is selected from: a condition ameliorated by increasing the secretion of an incretin, and a condition ameliorated by increasing a blood incretin level; and the incretin is GIP.

In some embodiments, the disorder is selected from: a condition ameliorated by increasing the secretion of an incretin, and a condition ameliorated by increasing a blood incretin level; and the incretin is PYY.

In some embodiments, the disorder is a condition ameliorated by increasing the secretion of an incretin; and the incretin is GLP-1.

In some embodiments, the disorder is a condition ameliorated by increasing the secretion of an incretin; and the incretin is GIP.

In some embodiments, the disorder is a condition ameliorated by increasing the secretion of an incretin; and the incretin is PYY.

In some embodiments, the disorder is a condition ameliorated by increasing a blood incretin level; and the incretin is GLP-1.

In some embodiments, the disorder is a condition ameliorated by increasing a blood incretin level; and the incretin is GIP.

In some embodiments, the disorder is a condition ameliorated by increasing a blood incretin level; and the incretin is PYY.

In some embodiments, the disorder is a condition characterized by low bone mass.

In some embodiments, the disorder is a condition characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

In some embodiments, the disorder is osteoporosis.

In some embodiments, the disorder is a neurological disorder.

In some embodiments, the disorder is a neurological disorder selected from: stroke and Parkinsonism.

In some embodiments, the disorder is a metabolic-related disorder.

In some embodiments, the disorder is a metabolic-related disorder selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

In some embodiments, the disorder is type 2 diabetes.

In some embodiments, the disorder is hyperglycemia.

In some embodiments, the disorder is hyperlipidemia.

In some embodiments, the disorder is hypertriglyceridemia.

In some embodiments, the disorder is type 1 diabetes.

In some embodiments, the disorder is dyslipidemia.

In some embodiments, the disorder is syndrome X.

In some embodiments, the disorder is obesity.

In some embodiments, the first pharmaceutical agent and the second pharmaceutical agent are provided in amounts which give a synergistic effect in treating the disorder.

In some embodiments, the amount of the first pharmaceutical agent alone is substantially therapeutically ineffective at treating the disorder.

In some embodiments, the amount of the second pharmaceutical agent alone is substantially therapeutically ineffective at treating the disorder.

In some embodiments, the first pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the first pharmaceutical agent is selected from: a DPP-IV inhibitor, a biguanide, an alpha-glucosidase inhibitor, a sulfonylurea, an SGLT2 inhibitor, and a meglitinide.

In some embodiments, the first pharmaceutical agent is a DPP-IV inhibitor.

In some embodiments, the first pharmaceutical agent is a biguanide.

In some embodiments, the first pharmaceutical agent is an alpha-glucosidase inhibitor.

In some embodiments, the first pharmaceutical agent is a sulfonylurea.

In some embodiments, the first pharmaceutical agent is an SGLT2 inhibitor.

In some embodiments, the first pharmaceutical agent is a meglitinide.

In some embodiments, the first pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.
In some embodiments, the first pharmaceutical agent is a biguanide selected from the following biguanides and pharmaceutically acceptable salts, solvates, and hydrates thereof:

(phenylethyl)biguanide;

dimethylbiguanide;

butylbiguanide; and

1-(p-chlorophenyl)-5-isopropylbiguanide.

In some embodiments, the first pharmaceutical agent is an alpha-glucosidase inhibitor selected from the following alpha-glucosidase inhibitors and pharmaceutically acceptable salts, solvates, and hydrates thereof:
(2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
(2R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the first pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the first pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the first pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

In some embodiments, the second pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the second pharmaceutical agent is selected from: a DPP-IV inhibitor, a biguanide, an alpha-glucosidase inhibitor, a sulfonylurea, an SGLT2 inhibitor, and a meglitinide.

In some embodiments, the second pharmaceutical agent is a DPP-IV inhibitor.

In some embodiments, the second pharmaceutical agent is a biguanide.

In some embodiments, the second pharmaceutical agent is an alpha-glucosidase inhibitor.

In some embodiments, the second pharmaceutical agent is a sulfonylurea.

In some embodiments, the second pharmaceutical agent is an SGLT2 inhibitor.

In some embodiments, the second pharmaceutical agent is a meglitinide.

In some embodiments, the second pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;

2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-ylboronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.

In some embodiments, the second pharmaceutical agent is a biguanide selected from the following biguanides and pharmaceutically acceptable salts, solvates, and hydrates thereof:

(phenylethyl)biguanide;

dimethylbiguanide;

butylbiguanide; and

1-(p-chlorophenyl)-5-isopropylbiguanide.

In some embodiments, the second pharmaceutical agent is an alpha-glucosidase inhibitor selected from the following alpha-glucosidase inhibitors and pharmaceutically acceptable salts, solvates, and hydrates thereof:
(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
(2R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the second pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the second pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the second pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

One aspect of the present invention pertains to methods for weight management, comprising administering to an individual in need thereof, a first pharmaceutical agent selected from a compound of the present invention; and a second pharmaceutical agent.

In some embodiments, the weight management comprises weight loss. In some embodiments, the weight management comprises maintenance of weight loss. In some embodiments, the weight management further comprises a reduced-calorie diet. In some embodiments, the weight management further comprises a program of regular exercise. In some embodiments, the weight management further comprises both a reduced-calorie diet and a program of regular exercise.

In some embodiments, the individual in need of weight management is a patient with an initial body mass of index ≧40 kg/m²; ≧39 kg/m²; ≧38 kg/m²; ≧37 kg/m²; ≧36 kg/m²; ≧35 kg/m²; ≧34 kg/m²; ≧33 kg/m²; ≧32 kg/m²; ≧31 kg/m²; ≧30 kg/m²; ≧29 kg/m²; ≧28 kg/m²; ≧27 kg/m²; ≧26 kg/m²; ≧25 kg/m²; ≧24 kg/m²; ≧23 kg/m²; ≧22 kg/m²; ≧21 kg/m²; or ≧20 kg/m²; and the patient optionally has at least one or at least two weight related comorbid condition(s).

In some embodiments, the comorbid condition(s) when present are selected from: hypertension, dyslipidemia, cardiovascular disease, glucose intolerance, and sleep apnea.

### Indications

In the context of the present invention, a compound as described herein, or a composition or pharmaceutical composition thereof, can be utilized for modulating the activity of GPR119-receptor-related diseases, conditions and/or disorders as described herein.

In some embodiments, modulating the activity includes the treatment of a GPR119-receptor-related disorder. In some embodiments, the GPR119-receptor-related disorder is a condition ameliorated by increasing a blood incretin level. In some embodiments, the GPR119-receptor-related disorder is a condition characterized by low bone mass. In some embodiments, the GPR119-receptor-related disorder is a neurological disorder. In some embodiments, the GPR119-receptor-related disorder is a metabolic-related disorder. In some embodiments, the GPR119-receptor-related disorder is type 2 diabetes. In some embodiments, the GPR119-receptor-related disorder is obesity.

Some embodiments of the present invention include every combination of one or more conditions characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

In some embodiments, the neurological disorder selected from: stroke and Parkinsonism.

Some embodiments of the present invention include every combination of one or more metabolic-related disorders selected from: type 1 diabetes, type 2 diabetes mellitus, and conditions associated therewith, such as, but not limited to, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertriglyceridemia, insulin resistance, impaired glucose metabolism, erectile dysfunction, skin and connective tissue disorders, foot ulcerations and UC, endothelial dysfunction and impaired vascular compliance.

Some embodiments of the present invention include every combination of one or more metabolic-related disorders selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

In some embodiments, the disorder is type 2 diabetes. In some embodiments, the disorder is hyperglycemia. In some embodiments, the disorder is hyperlipidemia. In some embodiments, the disorder is hypertriglyceridemia. In some embodiments, the disorder is type 1 diabetes. In some embodiments, the disorder is dyslipidemia. In some embodiments, the disorder is syndrome X. In some embodiments, the disorder is obesity. In some embodiments, the disorder is metabolic syndrome.

The term “metabolic syndrome” as used herein, refers to a set of risk factors that make a patient more susceptible to cardiovascular disease and/or type 2 diabetes. An individual is referred to having metabolic syndrome if the individual simultaneously has three or more of the following five risk factors as set forth by the American Heart Association and the National Heart, Lung, and Blood Institute: (1) elevated waist circumference: men—equal to or greater than 40 inches (102 cm), women—equal to or greater than 35 inches (88 cm); (2) elevated triglycerides: equal to or greater than 150 mg/dL; (3) reduced HDL (“good”) cholesterol: men—less than 40 mg/dL, women—less than 50 mg/dL; (4) elevated blood pressure: equal to or greater than 130/85 mm Hg; and (5) elevated fasting glucose: equal to or greater than 100 mg/dL.

### Compositions and Formulations

In any of the embodiments that recites the terms “first pharmaceutical agent selected from a compound of the present invention” and “second pharmaceutical agent”, it is appreciated that the term “second pharmaceutical agent” may in some aspects be further limited to a pharmaceutical agent that is not Compound 1 or a pharmaceutically acceptable salt, solvate, or hydrate thereof, and may refer to a pharmaceutical agent that is not detectable or has an EC₅₀ that is greater than a value selected from: 50 μM, 10 μM, 1 μM, and 0.1 μM in a GPR119 receptor activity assay as described in Example 4.

In any of the embodiments that recites the terms “first pharmaceutical agent” and “second pharmaceutical agent selected from a compound of the present invention”, it is appreciated that the term “first pharmaceutical agent” may in some aspects be further limited to a pharmaceutical agent that is not Compound 1 or a pharmaceutically acceptable salt, solvate, or hydrate thereof, and may refer to a pharmaceutical agent that is not detectable or has an EC₅₀ that is greater than a value selected from: 50 μM, 10 μM, 1 μM, and 0.1 μM in a GPR119 receptor activity assay as described in Example 4.

One aspect of the present invention pertains to compositions comprising a compound of the present invention.

One aspect of the present invention pertains to compositions comprising a compound of the present invention and a pharmaceutically acceptable carrier.

One aspect of the present invention pertains to compositions obtained by a method of the present invention.

One aspect of the present invention pertains to compositions comprising: a first pharmaceutical agent selected from a compound of the present invention; and a second pharmaceutical agent.

One aspect of the present invention pertains to compositions comprising: a first pharmaceutical agent selected from a compound of the present invention; a second pharmaceutical agent; and a pharmaceutically acceptable carrier.

In some embodiments, the first pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the first pharmaceutical agent is selected from: a DPP-IV inhibitor, a biguanide, an alpha-glucosidase inhibitor, a sulfonylurea, an SGLT2 inhibitor, and a meglitinide.

In some embodiments, the first pharmaceutical agent is a DPP-IV inhibitor.

In some embodiments, the first pharmaceutical agent is a biguanide.

In some embodiments, the first pharmaceutical agent is an alpha-glucosidase inhibitor.

In some embodiments, the first pharmaceutical agent is a sulfonylurea.

In some embodiments, the first pharmaceutical agent is an SGLT2 inhibitor.

In some embodiments, the first pharmaceutical agent is a meglitinide.

In some embodiments, the first pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.

In some embodiments, the first pharmaceutical agent is a biguanide selected from the following biguanides and pharmaceutically acceptable salts, solvates, and hydrates thereof:

(phenylethyl)biguanide;

dimethylbiguanide;

butylbiguanide; and
1-(p-chlorophenyl)-5-isopropylbiguanide.

In some embodiments, the first pharmaceutical agent is an alpha-glucosidase inhibitor selected from the following alpha-glucosidase inhibitors and pharmaceutically acceptable salts, solvates, and hydrates thereof:
(2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
(2R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the first pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the first pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the first pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

In some embodiments, the second pharmaceutical agent is selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue.

In some embodiments, the second pharmaceutical agent is selected from: a DPP-IV inhibitor, a biguanide, an alpha-glucosidase inhibitor, a sulfonylurea, an SGLT2 inhibitor, and a meglitinide.

In some embodiments, the second pharmaceutical agent is a DPP-IV inhibitor.

In some embodiments, the second pharmaceutical agent is a biguanide.

In some embodiments, the second pharmaceutical agent is an alpha-glucosidase inhibitor.

In some embodiments, the second pharmaceutical agent is a sulfonylurea.

In some embodiments, the second pharmaceutical agent is an SGLT2 inhibitor.

In some embodiments, the second pharmaceutical agent is a meglitinide.

In some embodiments, the second pharmaceutical agent is a DPP-IV inhibitor selected from the following DPP-IV inhibitors and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;

8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3S)-3-(1H-1,2,4-triazol-1-ylmethyl)cyclopentylamino]acetyl]pyrrolidine-2(S)-carbonitrile;

1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
(2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.

In some embodiments, the second pharmaceutical agent is a biguanide selected from the following biguanides and pharmaceutically acceptable salts, solvates, and hydrates thereof:

(phenylethyl)biguanide;

dimethylbiguanide;

butylbiguanide; and

1-(p-chlorophenyl)-5-isopropylbiguanide.

In some embodiments, the second pharmaceutical agent is an alpha-glucosidase inhibitor selected from the following alpha-glucosidase inhibitors and pharmaceutically acceptable salts, solvates, and hydrates thereof:
(2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal;
(2R,3R,4R,5S)-1-(2-hydroxyethyl)-2-(hydroxymethyl)piperidine-3,4,5-triol; and
(1S,2S,3R,4S,5S)-5-(1,3-dihydroxypropan-2-ylamino)-1-(hydroxymethyl)cyclohexane-1,2,3,4-tetraol.

In some embodiments, the second pharmaceutical agent is a sulfonylurea selected from the following sulfonylureas and pharmaceutically acceptable salts, solvates, and hydrates thereof:
N-(4-(N-(cyclohexylcarbamoyl)sulfamoyl)phenethyl)-5-methylpyrazine-2-carboxamide);
5-chloro-N-(4-(N-(cyclohexylcarbamoyl)sulfamoyl)phenethyl)-2-methoxybenzamide; and
3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide.

In some embodiments, the second pharmaceutical agent is an SGLT2 inhibitor selected from the following SGLT2 inhibitors and pharmaceutically acceptable salts, solvates, and hydrates thereof:
(2S,3R,4R,5S,6R)-2-(4-chloro-3-(4-ethoxybenzyl)phenyl)-6-(hydroxymethyl)tetrahydro-2H-pyran-3,4,5-triol;
ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate; and
ethyl ((2R,3S,4S,5R,6S)-3,4,5-trihydroxy-6-(2-(4-methoxybenzyl)phenoxy)tetrahydro-2H-pyran-2-yl)methyl carbonate.

In some embodiments, the second pharmaceutical agent is a meglitinide selected from the following meglitinides and pharmaceutically acceptable salts, solvates, and hydrates thereof:
(S)-2-ethoxy-4-(2-(3-methyl-1-(2-(piperidin-1-yl)phenyl)butylamino)-2-oxoethyl)benzoic acid;
(R)-2-((1r,4R)-4-isopropylcyclohexanecarboxamido)-3-phenylpropanoic acid; and
(S)-2-benzyl-4-((3aR,7aS)-1H-isoindol-2(3H,3aH,4H,5H,6H,7H,7aH)-yl)-4-oxobutanoic acid.

One aspect of the present invention pertains to compositions, methods, pharmaceutical products, uses, compounds, and pharmaceutical agents of the present invention, wherein said pharmaceutical agent or said second pharmaceutical agent is an inhibitor of DPP-IV selected from the following inhibitor of DPP-IV and pharmaceutically acceptable salts, solvates, and hydrates thereof: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate, hydrate or physiologically functional derivative thereof can be used as active ingredients in pharmaceutical compositions, specifically as GPR119 receptor modulators. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt, solvate, or hydrate of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active compound. A representative amount in a powder or tablet may contain from 0.5% to about 90% of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents.

Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

Compounds of the present invention may also be administered via a rapid dissolving or a slow release composition, wherein the composition includes a biodegradable rapid dissolving or slow release carrier (such as a polymer carrier and the like) and a compound of the invention. Rapid dissolving or slow release carriers are well known in the art and are used to form complexes that capture therein an active compound(s) and either rapidly or slowly degrade/dissolve in a suitable environment (e.g., aqueous, acidic, basic, etc). Such particles are useful because they degrade/dissolve in body fluids and release the active compound(s) therein. The particle size of a compound of the present invention, carrier or any excipient used in such a composition may be optimally adjusted using techniques known to those of ordinary skill in the art.

Particle size can play an important role in formulation. Reducing the size of the particles can be used to modify the physical characteristics. Particle size reduction increases both the number of particles and the amount of surface area per unit of volume. The increased surface area can improve the rate of solvation and therefore solubility. In addition, particle size reduction can improve gastrointestinal absorption for less soluble compounds. Particle size reduction can be obtained by any of the methods know in the art, for example, precipitation/crystallization, comminution (size reduction by a mechanical process), and the like, see for example Remington, The Science and Practice of Pharmacy, 20^{th} Edition, 2000, Lippincott Williams & Wilkins, (Editors: Gennaro et al.).

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds of the present invention which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent.

The compounds of this invention may form solvates with standard low molecular weight solvents using methods known to the skilled artisan. Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the GPR119 receptor modulators are utilized as active ingredients in pharmaceutical compositions, these are not intended for use in humans only, but in non-human mammals as well. Recent advances in the area of animal health-care mandate that consideration be given for the use of active agents, such as GPR119 receptor modulators, for the treatment of a GPR119 receptor-associated disease or disorder in companionship animals (e.g., cats, dogs, etc.) and in livestock animals (e.g., horses, cows, chickens, fish, etc.) Those of ordinary skill in the art are readily credited with understanding the utility of such compounds in such settings.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to salts described herein, it embraces pharmaceutically acceptable solvates and/or hydrates of such salts.

It will be apparent to those skilled in the art that the dosage forms described herein may comprise, as the active component, either a compound described herein or a pharmaceutically acceptable salt or as a pharmaceutically acceptable solvate or hydrate thereof. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present invention pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Polymorphs and Pseudopolymorphs

Polymorphism is the ability of a substance to exist as two or more crystalline phases that have different arrangements and/or conformations of the molecules in the crystal lattice. Polymorphs show the same properties in the liquid or gaseous state but they behave differently in the solid state.

Besides single-component polymorphs, drugs can also exist as salts and other multicomponent crystalline phases. For example, solvates and hydrates may contain an API host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily.

By way of example, Stahly recently published a polymorph screen of 245 compounds consisting of a “wide variety of structural types” that revealed about 90% of the compounds exhibited multiple solid forms. Overall, approximately half the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026.)

### Combination Therapy

A compound of the invention can be administered as the sole active pharmaceutical agent (i.e., mono-therapy), or it can be used in combination with one or more pharmaceutical agents (i.e., combination-therapy), such as pharmaceutical agents, such as, known anti-diabetic agents, either administered together or separately for the treatment of the diseases, conditions, and disorders described herein. Therefore, another aspect of the present invention includes methods of treatment of a metabolic related disorder, including a weight-related disorder, such as obesity, comprising administering to an individual in need thereof a therapeutically effective amount of Compound 1 or a pharmaceutically acceptable salt, solvate, or hydrate thereof, in combination with one or more pharmaceutical agents, such as anti-diabetic agents, as described herein.

In accordance with the present invention, the combination can be used by mixing the respective active components, Compound 1 or a pharmaceutically acceptable salt, solvate, or hydrate thereof and a pharmaceutical agent, either together or independently optionally with a physiologically acceptable carrier, excipient, binder, diluent, etc., as described herein, and administering the mixture or mixtures either orally or non-orally as a pharmaceutical composition(s). When a compound of the present invention is administered as a combination therapy with another active compound, the compound of the present invention and the pharmaceutical agent can be formulated as separate pharmaceutical compositions given at the same time or at different times; or the compound of the present invention and the pharmaceutical agent can be formulated together as a single unit dosage.

One aspect of the present invention pertains to first pharmaceutical agents selected from a compound according of the present invention, for use in combination with a second pharmaceutical agent for use in a method of treatment of the human or animal body by therapy.

One aspect of the present invention pertains to first pharmaceutical agents selected from a compound according of the present invention, for use in combination with a second pharmaceutical agent for use in a method of modulating the activity of a GPR119 receptor in an individual.

One aspect of the present invention pertains to first pharmaceutical agents selected from a compound according of the present invention, for use in combination with a second pharmaceutical agent for use in a method of agonizing a GPR119 receptor in an individual.

One aspect of the present invention pertains to first pharmaceutical agents selected from a compound according of the present invention, for use in combination with a second pharmaceutical agent for use in a method of increasing the secretion of an incretin in an individual.

One aspect of the present invention pertains to first pharmaceutical agents selected from a compound according of the present invention, for use in combination with a second pharmaceutical agent for use in a method of increasing a blood incretin level in an individual.

One aspect of the present invention pertains to first pharmaceutical agents selected from a compound according of the present invention, for use in combination with a second pharmaceutical agent for use in a method of treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

One aspect of the present invention pertains to first pharmaceutical agents for use in combination with a second pharmaceutical agent selected from a compound of the present invention, for use in a method of treatment of the human or animal body by therapy.

One aspect of the present invention pertains to first pharmaceutical agents for use in combination with a second pharmaceutical agent selected from a compound of the present invention, for use in a method of modulating the activity of a GPR119 receptor in an individual.

One aspect of the present invention pertains to first pharmaceutical agents for use in combination with a second pharmaceutical agent selected from a compound of the present invention, for use in a method of agonizing a GPR119 receptor in an individual.

One aspect of the present invention pertains to first pharmaceutical agents for use in combination with a second pharmaceutical agent selected from a compound of the present invention, for use in a method of increasing the secretion of an incretin in an individual.

One aspect of the present invention pertains to first pharmaceutical agents for use in combination with a second pharmaceutical agent selected from a compound of the present invention, for use in a method of increasing a blood incretin level in an individual.

One aspect of the present invention pertains to first pharmaceutical agents for use in combination with a second pharmaceutical agent selected from a compound of the present invention, for use in a method of treatment of a disorder selected from: a GPR119-receptor-related disorder; a condition ameliorated by increasing the secretion of an incretin; a condition ameliorated by increasing a blood incretin level; a condition characterized by low bone mass; a neurological disorder; a metabolic-related disorder; and obesity; in an individual.

In some embodiments, the method comprises administering the first pharmaceutical agent and the second pharmaceutical agent simultaneously, separately, or sequentially.

In some embodiments, the method comprises administering the first pharmaceutical agent and the second pharmaceutical agent simultaneously.

In some embodiments, the method comprises administering the first pharmaceutical agent and the second pharmaceutical agent separately.

In some embodiments, the method comprises administering the first pharmaceutical agent and the second pharmaceutical agent sequentially.

In some embodiments, the first pharmaceutical agent and the second pharmaceutical agent are provided in amounts which give a synergistic effect in treating the disorder.

In some embodiments, the amount of the first pharmaceutical agent alone is substantially therapeutically ineffective at treating the disorder.

In some embodiments, the amount of the second pharmaceutical agent alone is substantially therapeutically ineffective at treating the disorder.

Suitable pharmaceutical agents that can be used in combination with the compounds of the present invention include anti-obesity agents such as apolipoprotein-B secretion/microsomal triglyceride transfer protein (apo-B/MTP) inhibitors; MCR-4 agonists, cholecystokinin-A (CCK-A) agonists; serotonin and norepinephrine reuptake inhibitors (for example, sibutramine); sympathomimetic agents; β3 adrenergic receptor agonists; dopamine agonists (for example, bromocriptine); melanocyte-stimulating hormone receptor analogues; cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide]; melanin concentrating hormone antagonists; leptin (the OB protein); leptin analogues; leptin receptor agonists; galanin antagonists; lipase inhibitors (such as tetrahydrolipstatin, i.e., orlistat); anorectic agents (such as a bombesin agonist); neuropeptide-Y antagonists; thyromimetic agents; dehydroepiandrosterone or an analogue thereof; glucocorticoid receptor agonists or antagonists; orexin receptor antagonists; urocortin binding protein antagonists; glucagon-like peptide-1 (GLP-1) receptor agonists; ciliary neurotrophic factors (such as Axokine™ available from Regeneron Pharmaceuticals, Inc., Tarrytown, N.Y. and Procter & Gamble Company, Cincinnati, Ohio); human agouti-related proteins (AGRP); ghrelin receptor antagonists; histamine 3 receptor (H3R) antagonists or inverse agonists; neuromedin U receptor agonists; noradrenergic anorectic agents (for example, phentermine, mazindol and the like); and appetite suppressants (for example, bupropion).

Other anti-obesity agents, including the agents set forth infra, are well known, or will be readily apparent in light of the instant disclosure, to one of ordinary skill in the art. In some embodiments, the anti-obesity agents are selected from the group consisting of orlistat, sibutramine, bromocriptine, ephedrine, leptin, and pseudoephedrine. In a further embodiment, compounds of the present invention and combination therapies are administered in conjunction with exercise and/or a calorie-controlled diet.

It is understood that the scope of combination-therapy of the compounds of the present invention with anti-obesity agents, anorectic agents, appetite suppressant and related agents is not limited to those listed above, but includes in principle any combination with any pharmaceutical agent or pharmaceutical composition useful for the treatment of overweight and obese individuals.

It is understood that the scope of combination-therapy of the compounds of the present invention with other pharmaceutical agents is not limited to those listed herein, supra or infra, but includes in principle any combination with any pharmaceutical agent or pharmaceutical composition useful for the treatment of diseases, conditions or disorders that are linked to metabolic related disorders.

Some embodiments of the present invention include methods of treatment of a disease, disorder, condition or complication thereof as described herein, comprising administering to an individual in need of such treatment a therapeutically effective amount or dose of a compound of the present invention in combination with at least one pharmaceutical agent selected from the group consisting of: sulfonylureas (for example, tolbutamide (Orinase); acetohexamide (Dymelor); tolazamide (Tolinase); chlorpropamide (Diabinese); glipizide (Glucotrol); glyburide (Diabeta, Micronase, Glynase); glimepiride (Amaryl); gliclazide (Diamicron); and sulfonylureas known in the art); meglitinides (for example, repaglinide (Prandin), nateglinide (Starlix), mitiglinide, and other meglitinides known in the art); biguanides (for example, phenformin, metformin, buformin, and biguanides known in the art); α-glucosidase inhibitors (for example, acarbose, miglitol, and α-glucosidase inhibitors known in the art); thiazolidinediones—peroxisome proliferators-activated receptor-γ (i.e., PPAR-γ) agonists (for example, rosiglitazone (Avandia), pioglitazone (Actos), troglitazone (Rezulin), rivoglitazone, ciglitazone, and thiazolidinediones known in the art); insulin and insulin analogues; anti-diabetic peptide analogues (for example, exenatide, liraglutide, taspoglutide, and anti-diabetic peptides analogues know in the art); HMG-CoA reductase inhibitors (for example, rosuvastatin, pravastatin and its sodium salt, simvastatin, lovastatin, atorvastatin, fluvastatin, cerivastatin, rosuvastatin, pitavastatin, pravastatin, and other HMG-CoA reductase inhibitors known in the art); cholesterol-lowering drugs (for example, fibrates that include: bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and other fibrates known in the art; bile acid sequestrants which include: cholestyramine, colestipol and the like; and niacin); antiplatelet agents (for example, aspirin and adenosine diphosphate receptor antagonists that include: clopidogrel, ticlopidine and the like); angiotensin-converting enzyme inhibitors (for example, captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and other angiotensin converting enzyme inhibitors known in the art); angiotensin II receptor antagonists [for example, losartan (and the potassium salt form), and other angiotensin II receptor antagonists known in the art; adiponectin; squalene synthesis inhibitors {for example, (S)-α-[bis[2,2-dimethyl-1-oxopropoxy)methoxy]phosphinyl]-3-phenoxybenzenebutanesulfonic acid, mono potassium salt (BMS-188494) and other squalene synthesis inhibitors known in the art}; and the like. In some embodiments, compounds of the present invention and the pharmaceutical agents are administered separately. In further embodiments, compounds of the present invention and the pharmaceutical agents are administered simultaneously.

Suitable pharmaceutical agents that can be used in conjunction with compounds of the present invention include, but are not limited to: amylin agonists (for example, pramlintide); insulin secretagogues (for example, GLP-1 agonists, exendin-4, and insulinotropin (NN2211)); acyl CoA cholesterol acetyltransferase inhibitors (for example, ezetimibe, eflucimibe, and other acyl CoA cholesterol acetyltransferase inhibitors known in the art); cholesterol absorption inhibitors (for example, ezetimibe, pamaqueside and other cholesterol absorption inhibitors known in the art); cholesterol ester transfer protein inhibitors (for example, CP-529414, JTT-705, CETi-1, and other cholesterol ester transfer protein inhibitors known in the art); microsomal triglyceride transfer protein inhibitors (for example, implitapide, and other microsomal triglyceride transfer protein inhibitors known in the art); cholesterol modulators (for example, NO-1886, and other cholesterol modulators known in the art); bile acid modulators (for example, GT103-279 and other bile acid modulators known in the art); insulin signaling pathway modulators; inhibitors of protein tyrosine phosphatases (PTPases); non-small molecule mimetics and inhibitors of glutamine-fructose-6-phosphate amidotransferase (GFAT); compounds influencing a dysregulated hepatic glucose production; inhibitors of glucose-6-phosphatase (G6 Pase); inhibitors of fructose-1,6-bisphosphatase (F-1,6-BPase); inhibitors of glycogen phosphorylase (GP); glucagon receptor antagonists; inhibitors of phosphoenolpyruvate carboxykinase (PEPCK); pyruvate dehydrogenase kinase (PDHK) inhibitors; insulin sensitivity enhancers; insulin secretion enhancers; inhibitors of gastric emptying; α₂-adrenergic antagonists; retinoid X receptor (RXR) agonists; and DPP-IV inhibitors; and the like.

### Tripartite Combinations

Some aspects of the present invention include compounds of the present invention that can be employed in any of the methods, pharmaceutical products, uses, compounds, and pharmaceutical agents, as described herein, in combination with two distinct pharmaceutical agents.

In some embodiments, the two distinct pharmaceutical agents are selected from any of the pharmaceutical agents, or classes of pharmaceutical agents described herein. In some embodiments, the two distinct pharmaceutical agents are selected from: a DPP-IV inhibitor, a biguanide, an alpha-glucosidase inhibitor, an insulin analogue, a sulfonylurea, an SGLT2 inhibitor, a meglitinide, a thiazolidinedione, and an anti-diabetic peptide analogue. In some embodiments, the two distinct pharmaceutical agents include every combination selected from pharmaceutical agents of the following group: a DPP-IV inhibitor, a biguanide, an alpha-glucosidase inhibitor, a sulfonylurea, and an SGLT2 inhibitor.

Some embodiments of the present invention include every combination of one or more compounds selected from compounds of the following group and pharmaceutically acceptable salts, solvates, and hydrates thereof: a DPP-IV inhibitor selected from: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3(R)-aminopiperidin-1-yl]-3-methyl-2,4-dioxo-1,2,3,4-tetrahydropyrimidin-1-ylmethyl]benzonitrile; 8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl) ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5difluoropiperidin-2-one; (R)-2-((6-(3-aminopiperidin-1-yl)-3-methyl-2,4-dioxo-3,4-dihydropyrimidin-1(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile; (2S)-1-{[2-(5-methyl-2-phenyl-oxazol-4-yl)-ethylamino]-acetyl}-pyrrolidine-2-carbonitrile; (2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine; a biguanide selected from: phenformin ((phenylethyl)biguanide); metformin (dimethylbiguanide); buformin (butylbiguanide); and proguanil (1-(p-chlorophenyl)-5-isopropylbiguanide); an α-glucosidase inhibitor selected from: acarbose ((2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal); miglitol ((2R,3R,4R,5S)-1-(2-hydroxyethyl)-2-(hydroxymethyl)piperidine-3,4,5-triol); and voglibose ((1S,2S,3R,4S,5S)-5-(1,3-dihydroxypropan-2-ylamino)-1-(hydroxymethyl)cyclohexane-1,2,3,4-tetraol); an insulin analogue selected from: NPH insulin (also known as Humulin N, Novolin N, NPH Lletin II, and insulin isophane); insulin lispro (28B-L-lysine-29B-L-proline-insulin, wherein insulin is human insulin); insulin aspart (28B-L-aspartic acid-insulin, wherein insulin is human insulin); and insulin glulisine (3B-L-lysine-29B-L-glutamic acid-insulin, wherein insulin is human insulin); a sulfonylurea selected from: tolbutamide (Orinase, N-(butylcarbamoyl)-4-methylbenzenesulfonamide); acetohexamide (Dymelor, 4-acetyl-N-(cyclohexylcarbamoyl)benzenesulfonamide); tolazamide (Tolinase, N-(azepan-1-ylcarbamoyl)-4-methylbenzenesulfonamide); chlorpropamide (Diabinese, 4-chloro-N-(propylcarbamoyl)benzenesulfonamide); glipizide (Glucotrol, N-(4-(N-(cyclohexylcarbamoyl)sulfamoyl)phenethyl)-5-methylpyrazine-2-carboxamide); glibenclamide, also known as glyburide (Diabeta, Micronase, Glynase, 5-chloro-N-(4-(N-(cyclohexylcarbamoyl)sulfamoyl)phenethyl)-2-methoxybenzamide); glimepiride (Amaryl, 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide); and gliclazide (Diamicron, N-(hexahydrocyclopenta[c]pyrrol-2(1H)-ylcarbamoyl)-4-methylbenzenesulfonamide); an SGLT2 inhibitor selected from: dapagliflozin ((2S,3R,4R,5S,6R)-2-(4-chloro-3-(4-ethoxybenzyl)phenyl)-6-(hydroxymethyl)tetrahydro-2H-pyran-3,4,5-triol); remogliflozin (ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate); ASP1941, canagliflozin ((2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol); ISIS 388626; sergliflozin (ethyl ((2R,3S,4S,5R,6S)-3,4,5-trihydroxy-6-(2-(4-methoxybenzyl)phenoxy)tetrahydro-2H-pyran-2-yl)methyl carbonate), AVE2268 ((2R,3S,4S,5R,6S)-2-(hydroxymethyl)-6-(2-(4-methoxybenzyl)thiophen-3-yloxy)tetrahydro-2H-pyran-3,4,5-triol), BI10773, CSG453; and LX4211; a meglitinide selected from: repaglinide (Prandin, (S)-2-ethoxy-4-(2-(3-methyl-1-(2-(piperidin-1-yl)phenyl)butylamino)-2-oxoethyl)benzoic acid); nateglinide (Starlix, (R)-2-((1r,4R)-4-isopropylcyclohexanecarboxamido)-3-phenylpropanoic acid); and mitiglinide ((S)-2-benzyl-4-((3aR,7aS)-1H-isoindol-2(3H,3aH,4H,5H,6H,7H,7aH)-yl)-4-oxobutanoic acid); a thiazolidinedione selected from: rosiglitazone (Avandia, 5-(4-(2-(methyl(pyridin-2-yl)amino)ethoxy)benzyl)thiazolidine-2,4-dione); pioglitazone (Actos, 5-(4-(2-(5-ethylpyridin-2-yl)ethoxy)benzyl)thiazolidine-2,4-dione); troglitazone (Rezulin, 5-(4-((6-hydroxy-2,5,7,8-tetramethylchroman-2-yl)methoxy)benzyl)thiazolidine-2,4-dione); rivoglitazone (5-(4-((6-methoxy-1-methyl-1H-benzo[d]imidazol-2-yl)methoxy)benzyl)thiazolidine-2,4-dione); and ciglitazone (5-(4-((1-methylcyclohexyl)methoxy)benzyl)thiazolidine-2,4-dione); and an anti-diabetic peptide analogue selected from: exenatide; liraglutide; and taspoglutide.

In some embodiments, the two distinct pharmaceutical agents include every combination selected from pharmaceutical agents of the following group: sitagliptin, vildagliptin, saxagliptin, alogliptin, linagliptin, phenformin, metformin, buformin, acarbose, miglitol, voglibose, tolbutamide, acetohexamide, tolazamide, chlorpropamide, glipizide, glibenclamide, glimepiride, gliclazide, dapagliflozin, remogliflozin, and sergliflozin.

### Dipeptidyl Peptidase-IV Inhibitors

Dipeptidyl peptidase-IV (DPP-IV, EC 3.4.14.5) exhibits catalytic activity against a broad range of peptide substrates that includes peptide hormones, neuropeptides, and chemokines. The incretins glucagon-like peptide 1 (GLP-1), and glucose-dependent insulinotropic polypeptide (GIP), which stimulate glucose-dependent insulin secretion and otherwise promote blood glucose homeostasis, are rapidly cleaved by DPP-IV at the position-2 alanine leading to inactivation of their biological activity. Peptide YY (PYY) is a gut peptide that has been implicated in modulating satiety (Chaudhri et al., Annu Rev Physiol (2008) 70:239-255). PYY is released into the circulation as PYY₁₋₃₆ and PYY₃₋₃₆ (Eberlein et al., Peptides (1989) 10:797-803). PYY₃₋₃₆ is generated from PYY₁₋₃₆ by cleavage of the N-terminal Tyr and Pro residues by DPP-IV. Both pharmacological and genetic attenuation of DPP-IV activity is associated with enhanced incretin action, increased insulin, and lower blood glucose in vivo. Genetic attenuation of DPP-IV activity has been shown to provide resistance to obesity and to improve insulin sensitivity. DPP-IV inhibitors have shown to be useful as therapeutics, for example, oral administration of vildagliptin (1-[2-(3-hydroxyadamant-1-ylamino)acetyl]pyrrolidine-2(S)-carbonitrile) or sitagliptin (3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one) to human patients suffering with type 2 diabetes has been found to reduce fasting glucose and postprandial glucose excursion in association with significantly reduced HbA1 levels. For reviews on the application of DPP-IV inhibitors for the treatment of type 2 diabetes, reference is made to the following publications: (1) H.-U. Demuth, et al., “Type 2 diabetes-therapy with DPP-IV inhibitors,” Biochim. Biophys. Acta, 1751: 33-44 (2005), and (2) K. Augustyns, et al., “Inhibitors of proline-specific dipeptidyl peptidases: DPP-IV inhibitors as a novel approach for the treatment of type 2 diabetes,” Expert Opin. Ther. Patents, 15: 1387-1407 (2005).

Accordingly, suitable pharmaceutical agents include DPP-IV inhibitors that can be used in conjunction with compounds of the present invention either dosed separately or together. DPP-IV inhibitors are well-known in the art or can be readily identified and their in vitro biological activity determined using any number of methods available, for example, O'Brien, M., Daily, B., Schurria, M., “Assay for DPPIV activity using a homogeneous, luminescent method,” Cell Notes, Issue 11, 2005; see also the DPPIV-Glo™ Protease Assay Technical Bulletin #TB339.

Examples of DPP-IV inhibitors are described in Villhauer et al., J. Med. Chem. (2003) 46:2774-2789, for LAF237; Ahren et al., J. Clin. Endocrinol. Metab. (2004) 89:2078-2084; Villhauer et al., J. Med. Chem. (2002) 45:2362-2365 for NVP-DPP728; Ahren et al., Diabetes Care (2002) 25:869-875 for NVP-DPP728; Peters et al., Bioorg. Med. Chem. Lett. (2004) 14:1491-1493; Caldwell et al., Bioorg. Med. Chem. Lett. (2004) 14:1265-1268; Edmondson et al., Bioorg. Med. Chem. Lett. (2004) 14:5151-5155; and Abe et al., J. Nat Prod. (2004) 67:999-1004.

Specific examples of DPP-IV inhibitors include, but are not limited to, dipeptide derivatives or dipeptide mimetics such as alanine-pyrrolidide, isoleucine-thiazolidide, and the pseudosubstrate N-valyl prolyl, O-benzoyl hydroxylamine, as described, for example, in U.S. Pat. No. 6,303,661.

Some embodiments of the present invention include every combination of one or more DPP-IV inhibitors selected from the DPP-IV inhibitors found in U.S. Pat. Nos. 6,869,947, 6,867,205, 6,861,440, 6,849,622, 6,812,350, 6,803,357, 6,800,650, 6,727,261, 6,716,843, 6,710,040, 6,706,742, 6,645,995, 6,617,340, 6,699,871, 6,573,287, 6,432,969, 6,395,767, 6,380,398, 6,303,661, 6,242,422, 6,166,063, 6,100,234, and 6,040,145.

Some embodiments of the present invention include every combination of one or more DPP-IV inhibitors selected from the DPP-IV inhibitors found in U.S. Pat. Nos. 2,005,059724, 2005059716, 2005043292, 2005038020, 2005032804, 2005004205, 2004259903, 2004259902, 2004259883, 2004254226, 2004242898, 2004229926, 2004180925, 2004176406, 2004138214, 2004116328, 2004110817, 2004106656, 2004097510, 2004087587, 2004082570, 2004077645, 2004072892, 2004063935, 2004034014, 2003232788, 2003225102, 2003216450, 2003216382, 2003199528, 2003195188, 2003162820, 2003149071, 2003134802, 2003130281, 2003130199, 2003125304, 2003119750, 2003119738, 2003105077, 2003100563, 2003087950, 2003078247, 2002198205, 2002183367, 2002103384, 2002049164, and 2002006899.

Some embodiments of the present invention include every combination of one or more DPP-IV inhibitors selected from the DPP-IV inhibitors found in International Patent Application Publication Nos. WO2005/087235, WO2005/082348, WO2005/082849, WO2005/079795, WO2005/075426, WO2005/072530, WO2005/063750, WO2005/058849, WO2005/049022, WO2005/047297, WO2005/044195, WO2005/042488, WO2005/040095, WO2005/037828, WO2005/037779, WO2005/034940, WO2005/033099, WO2005/032590, WO2005/030751, WO2005/030127, WO2005/026148, WO2005/025554, WO2005/023762, WO2005/020920, WO05/19168, WO05/12312, WO05/12308, WO05/12249, WO05/11581, WO05/09956, WO05/03135, WO05/00848, WO05/00846, WO04/112701, WO04/111051, WO04/111041, WO04/110436, WO04/110375, WO04/108730, WO04/104216, WO04/104215, WO04/103993, WO04/103276, WO04/99134, WO04/96806, WO04/92128, WO04/87650, WO04/87053, WO04/85661, WO04/85378, WO04/76434, WO04/76433, WO04/71454, WO04/69162, WO04/67509, WO04/64778, WO04/58266, WO04/52362, WO04/52850, WO04/50022, WO04/50658, WO04/48379, WO04/46106, WO04/43940, WO04/41820, WO04/41795, WO04/37169, WO04/37181, WO04/33455, WO04/32836, WO04/20407, WO04/18469, WO04/18468, WO04/18467, WO04/14860, WO04/09544, WO04/07468, WO04/07446, WO04/04661, WO04/00327, WO03/106456, WO03/104229, WO03/101958, WO03/101448, WO03/99279, WO03/95425, WO03/84940, WO03/82817, WO03/80633, WO03/74500, WO03/72556, WO03/72528, WO03/68757, WO03/68748, WO03/57666, WO03/57144, WO03/55881, WO03/45228, WO03/40174, WO03/38123, WO03/37327, WO03/35067, WO03/35057, WO03/24965, WO03/24942, WO03/22871, WO03/15775, WO03/04498, WO03/04496, WO03/02530, WO03/02596, WO03/02595, WO03/02593, WO03/02553, WO03/02531, WO03/00181, WO03/00180, WO03/00250, WO02/83109, WO02/83128, WO02/76450, WO02/68420, WO02/62764, WO02/55088, WO02/51836, WO02/38541, WO02/34900, WO02/30891, WO02/30890, WO02/14271, WO02/02560, WO01/97808, WO01/96295, WO01/81337, WO01/81304, WO01/68603, WO01/55105, WO01/52825, WO01/34594, WO00/71135, WO00/69868, WO00/56297, WO00/56296, WO00/34241, WO00/23421, WO00/10549, WO99/67278, WO99/62914, WO99/61431, WO99/56753, WO99/25719, WO99/16864, WO98/50066, WO98/50046, WO98/19998, WO98/18763, WO97/40832, WO95/29691, WO95/15309, WO93/10127, WO93/08259, and WO91/16339.

Some embodiments of the present invention include every combination of one or more DPP-IV inhibitors selected from the DPP-IV inhibitors found in Patent Publication Nos. EP1517907, EP1513808, EP1492777, EP1490335, EP1489088, EP1480961, EP1476435, EP1476429, EP1469873, EP1465891, EP1463727, EP1461337, EP1450794, EP1446116, EP1442049, EP1441719, EP1426366, EP1412357, EP1406873, EP1406872, EP1406622, EP1404675, EP1399420, EP1399471, EP1399470, EP1399469, EP1399433, EP1399154, EP1385508, EP1377288, EP1355886, EP1354882, EP1338592, EP1333025, EP1304327, EP1301187, EP1296974, EP1280797, EP1282600, EP1261586, EP1258476, EP1254113, EP1248604, EP1245568, EP1215207, EP1228061, EP1137635, EP1123272, EP1104293, EP1082314, EP1050540, EP1043328, EP0995440, EP0980249, EP0975359, EP0731789, EP0641347, EP0610317, EP0528858, CA2466870, CA2433090, CA2339537, CA2289125, CA2289124, CA2123128, DD296075, DE19834591, DE19828113, DE19823831, DE19616486, DE10333935, DE10327439, DE10256264, DE10251927, DE10238477, DE10238470, DE10238243, DE10143840, FR2824825, FR2822826, JP2005507261, JP2005505531, JP2005502624, JP2005500321, JP2005500308, JP2005023038, JP2004536115, JP2004535445, JP2004535433, JP2004534836, JP2004534815, JP2004532220, JP2004530729, JP2004525929, JP2004525179, JP2004522786, JP2004521149, JP2004503531, JP2004315496, JP2004244412, JP2004043429, JP2004035574, JP2004026820, JP2004026678, JP2004002368, JP2004002367, JP2003535898, JP2003535034, JP2003531204, JP2003531191, JP2003531118, JP2003524591, JP2003520849, JP2003327532, JP2003300977, JP2003238566, JP2002531547, JP2002527504, JP2002517401, JP2002516318, JP2002363157, JP2002356472, JP2002356471, JP2002265439, JP2001510442, JP2000511559, JP2000327689, JP2000191616, JP1998182613, JP1998081666, JP1997509921, JP1995501078, and JP1993508624.

In some embodiments, the DPP-IV inhibitor has an IC₅₀ of less than about 10 μM, less than about 1 μM, less than about 100 nM, less than about 75 nM, less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM, in any one of the DPP-IV inhibition assays known in the art, including in the references disclosed herein. In some embodiments, the DPP-IV inhibitor has an IC₅₀ of less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM, in any one of the DPP-IV inhibition assays known in the art, including in the references disclosed herein.

In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 10-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 100-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 10-fold. In some embodiments, the DPP-IV inhibitor is a selective DPP-IV inhibitor, wherein the selective DPP-IV inhibitor has a selectivity for human plasma DPP-IV over one or more of PPCE, DPP-II, DPP-8 and DPP-9 of at least about 1000-fold.

In some embodiments, the DPP-IV inhibitor is orally active.

In some embodiments, the DPP-IV inhibitor is an inhibitor of human DPP-IV.

Some embodiments of the present invention include every combination of one or more compounds selected from compounds of the following group and pharmaceutically acceptable salts, solvates, and hydrates thereof: 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one; 1-[2-(3-hydroxyadamant-1-ylamino)acetyl]pyrrolidine-2(S)-carbonitrile; (1S,3S,5S)-2-[2(S)-amino-2-(3-hydroxyadamantan-1-yl)acetyl]-2-azabicyclo[3.1.0]hexane-3-carbonitrile; 2-[6-[3(R)-aminopiperidin-1-yl]-3-methyl-2,4-dioxo-1,2,3,4-tetrahydropyrimidin-1-ylmethyl]benzonitrile; 8-[3(R)-aminopiperidin-1-yl]-7-(2-butynyl)-3-methyl-1-(4-methylquinazolin-2-ylmethyl)xanthine; 1-[N-[3(R)-pyrrolidinyl]glycyl]pyrrolidin-2(R)-yl boronic acid; 4(S)-fluoro-1-[2-[(1R,3S)-3-(1H-1,2,4-triazol-1-ylmethyl)cyclopentylamino]acetyl]pyrrolidine-2(S)-carbonitrile; 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one; (2S,4S)-2-cyano-4-fluoro-1-[(2-hydroxy-1,1-dimethyl)ethylamino]acetylpyrrolidine; 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione; 1-((3S,4S)-4-amino-1-(4-(3,3-difluoropyrrolidin-1-yl)-1,3,5-triazin-2-yl)pyrrolidin-3-yl)-5,5-difluoropiperidin-2-one; (R)-2-((6-(3-aminopiperidin-1-yl)-3-methyl-2,4-dioxo-3,4-dihydropyrimidin-1(2H)-yl)methyl)-4-fluorobenzonitrile; 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide; ((2S,4S)-4-(4-(3-methyl-1-phenyl-1H-pyrazol-5-yl)piperazin-1-yl)pyrrolidin-2-yl)(thiazolidin-3-yl)methanone; (2S,4S)-1-[2-[(4-ethoxycarbonylbicyclo[2.2.2]oct-1-yl)amino]acetyl]-4-fluoropyrrolidine-2-carbonitrile; 6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione; 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile; (2S)-1-{[2-(5-methyl-2-phenyl-oxazol-4-yl)-ethylamino]-acetyl}-pyrrolidine-2-carbonitrile; (2S)-1-{[1,1-dimethyl-3-(4-pyridin-3-yl-imidazol-1-yl)-propylamino]-acetyl}-pyrrolidine-2-carbonitrile; (3,3-difluoropyrrolidin-1-yl)-((2S,4S)-4-(4-(pyrimidin-2-yl)piperazin-1-yl)pyrrolidin-2-yl)methanone; (2S,4S)-1-[(2S)-2-amino-3,3-bis(4-fluorophenyl)propanoyl]-4-fluoropyrrolidine-2-carbonitrile; (2S,5R)-5-ethynyl-1-{N-(4-methyl-1-(4-carboxy-pyridin-2-yl)piperidin-4-yl)glycyl}pyrrolidine-2-carbonitrile; and (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.

Sitagliptin phosphate (Januvia, MK-0431, dihydrogenphosphate salt of 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one) is marketed by Merck & Co. for once-daily oral treatment of type 2 diabetes. Januvia was first launched in Mexico followed by commercialization in the U.S. In 2007, the product was approved by the European Medicines Evaluation Agency (EMEA) and is currently available in the U.K., Germany and Spain. In 2009, Januvia was approved and launched in Japan. In addition, Merck has also filed for approval of Januvia in the U.S. as an adjunct to diet and exercise and in combination with other therapies to improve glycemic control in the treatment of diabetes. The compound, 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one, and pharmaceutically acceptable salts thereof are disclosed in international patent publication WO2003/004498. Some embodiments of the present invention include every combination of one or more compounds selected from compounds disclosed in WO2003/004498 and pharmaceutically acceptable salts, solvates, and hydrates thereof. In some embodiments, the DPP-IV inhibitor is selected from 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one, and pharmaceutically acceptable salts, solvates, and hydrates thereof:

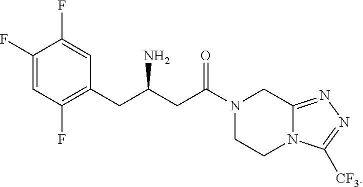 [SMILES: N[C@@H](CC(=O)N1CCn2c(nnc2C(F)(F)F)C1)Cc1cc(F)c(F)cc1F]

In some embodiments, the DPP-IV inhibitor is 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate:

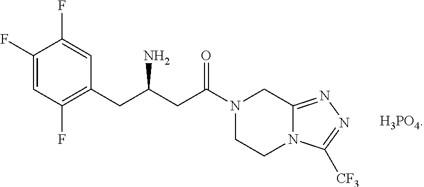

The crystalline form of 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate salt monohydrate is disclosed in international patent publication WO2005/003135. In some embodiments, the DPP-IV inhibitor is crystalline 3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one phosphate monohydrate.

Vildagliptin (Galvus®, LAF-237, 1-[2-(3-hydroxyadamant-1-ylamino)acetyl]pyrrolidine-2(S)-carbonitrile) is another DPP-IV inhibitor and was first commercialized in Brazil and Mexico by Novartis for oral, once-daily treatment of type 2 diabetes. In 2008, a marketing authorization application (MAA) was approved in the E.U. for this indication and launch took place in the U.K. in March, 2008. An approvable letter has been received for the regulatory application filed in the U.S. Vildagliptin was approved in Japan in 2010. The compound, 1-[2-(3-hydroxyadamant-1-ylamino)acetyl]pyrrolidine-2(S)-carbonitrile, is disclosed in international patent publication WO2000/034241. Some embodiments of the present invention include every combination of one or more compounds selected from compounds disclosed in WO2000/034241 and pharmaceutically acceptable salts, solvates, and hydrates thereof. In some embodiments, the DPP-IV inhibitor is selected from 1-[2-(3-hydroxyadamant-1-ylamino)acetyl]pyrrolidine-2(S)-carbonitrile, and pharmaceutically acceptable salts, solvates, and hydrates thereof:

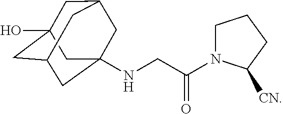 [SMILES: N#C[C@@H]1CCCN1C(=O)CNC12CC3CC(CC(O)(C3)C1)C2]

Certain salts of the compound, 1-[2-(3-hydroxyadamant-1-ylamino)acetyl]pyrrolidine-2(S)-carbonitrile, are disclosed in international patent publication WO2007/019255. In some embodiments, the DPP-IV inhibitor is 1-[2-(3-hydroxyadamant-1-ylamino)acetyl]pyrrolidine-2(S)-carbonitrile HCl:

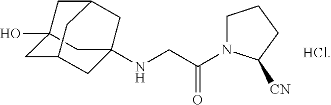 [SMILES: N#C[C@@H]1CCCN1C(=O)CNC12CC3CC(CC(O)(C3)C1)C2]

Saxagliptin (Onglyza™, BMS-477118, (1S,3S,5S)-2-[2(S)-amino-2-(3-hydroxyadamantan-1-yl)acetyl]-2-azabicyclo[3.1.0]hexane-3-carbonitrile) is another DPP-IV inhibitor, which was launched in 2009 by AstraZeneca and Bristol-Myers Squibb in the U.S. for the treatment of type 2 diabetes. In 2009, the product was approved in the E.U. for the treatment of type 2 diabetes independently or in combination with metformin. Phase 3 clinical studies are ongoing in Japan for the treatment of type 2 diabetes. The compound, (1S,3S,5S)-2-[2(S)-amino-2-(3-hydroxyadamantan-1-yl)acetyl]-2-azabicyclo[3.1.0]hexane-3-carbonitrile, is disclosed in international patent publication WO2001/068603. Some embodiments of the present invention include every combination of one or more compounds selected from compounds disclosed in WO2001/068603 and pharmaceutically acceptable salts, solvates, and hydrates thereof. In some embodiments, the DPP-IV inhibitor is selected from (1S,3S,5S)-2-[2(S)-amino-2-(3-hydroxyadamantan-1-yl)acetyl]-2-azabicyclo[3.1.0]hexane-3-carbonitrile, and pharmaceutically acceptable salts, solvates, and hydrates thereof:

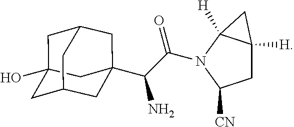

Takeda has filed for regulatory approval of the DPP-IV inhibitor, alogliptin (SYR-322, 2-[6-[3(R)-aminopiperidin-1-yl]-3-methyl-2,4-dioxo-1,2,3,4-tetrahydropyrimidin-1-ylmethyl]benzonitrile) in Japan and the U.S for the once-daily, oral treatment of type 2 diabetes. The compound, 2-[6-[3(R)-aminopiperidin-1-yl]-3-methyl-2,4-dioxo-1,2,3,4-tetrahydropyrimidin-1-ylmethyl]benzonitrile, and pharmaceutically acceptable salts thereof are disclosed in international patent publication WO2005/095381. Some embodiments of the present invention include every combination of one or more compounds selected from compounds disclosed in WO2005/095381 and pharmaceutically acceptable salts, solvates, and hydrates thereof. In some embodiments, the DPP-IV inhibitor is selected from 2-[6-[3(R)-aminopiperidin-1-yl]-3-methyl-2,4-dioxo-1,2,3,4-tetrahydropyrimidin-1-ylmethyl]benzonitrile, and pharmaceutically acceptable salts, solvates, and hydrates thereof:

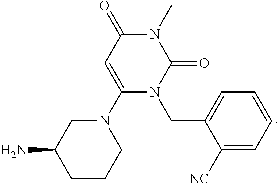 [SMILES: Cn1c(=O)cc(N2CCC[C@@H](N)C2)n(Cc2ccccc2C#N)c1=O]

The crystalline form of 2-[6-[3(R)-aminopiperidin-1-yl]-3-methyl-2,4-dioxo-1,2,3,4-tetrahydropyrimidin-1-ylmethyl]benzonitrile is disclosed in international patent publication WO2007/035372. In some embodiments, the DPP-IV inhibitor is 2-[6-[3(R)-aminopiperidin-1-yl]-3-methyl-2,4-dioxo-1,2,3,4-tetrahydropyrimidin-1-ylmethyl]benzonitrile benzoate:

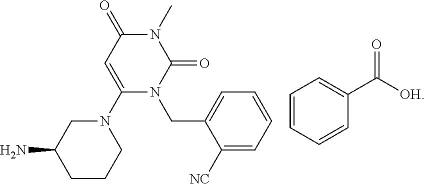

Linagliptin (BI-1356, Ondero®, 8-[3(R)-aminopiperidin-1-yl]-7-(2-butynyl)-3-methyl-1-(4-methylquinazolin-2-ylmethyl)xanthine) is a DPP-IV inhibitor in phase 3 clinical development at Boehringer Ingelheim to evaluate its potential as add-on therapy to metformin for the treatment of type 2 diabetes. The compound, 8-[3(R)-aminopiperidin-1-yl]-7-(2-butynyl)-3-methyl-1-(4-methylquinazolin-2-ylmethyl)xanthine, is disclosed in international patent publication WO2004/018468. Some embodiments of the present invention include every combination of one or more compounds selected from compounds disclosed in WO2004/018468 and pharmaceutically acceptable salts, solvates, and hydrates thereof. In some embodiments, the DPP-IV inhibitor is selected from 8-[3(R)-aminopiperidin-1-yl]-7-(2-butynyl)-3-methyl-1-(4-methylquinazolin-2-ylmethyl)xanthine, and pharmaceutically acceptable salts, solvates, and hydrates thereof:

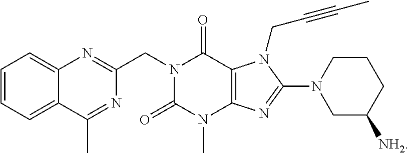 [SMILES: CC#CCn1c(N2CCC[C@@H](N)C2)nc2c1c(=O)n(Cc1nc(C)c3ccccc3n1)c(=O)n2C]

Certain polymorphs of the compound, 8-[3(R)-aminopiperidin-1-yl]-7-(2-butynyl)-3-methyl-1-(4-methylquinazolin-2-ylmethyl)xanthine, are disclosed in international patent publication WO2007/128721. In some embodiments, the DPP-IV inhibitor is a crystalline form of 8-[3(R)-aminopiperidin-1-yl]-7-(2-butynyl)-3-methyl-1-(4-methylquinazolin-2-ylmethyl)xanthine.

Dutogliptin (PHX-1149, 1-[N-[3(R)-pyrrolidinyl]glycyl]pyrrolidin-2(R)-yl boronic acid) is a DPP-IV inhibitor in phase 3 clinical trials by Phenomix and Forest for the oral, once-daily treatment of type 2 diabetes. The compound, 1-[N-[3(R)-pyrrolidinyl]glycyl]pyrrolidin-2(R)-yl boronic acid, and pharmaceutically acceptable salts thereof are disclosed in international patent publication WO2005/047297. Some embodiments of the present invention include every combination of one or more compounds selected from compounds disclosed in WO2005/047297 and pharmaceutically acceptable salts, solvates, and hydrates thereof. In some embodiments, the DPP-IV inhibitor is selected from 1-[N-[3(R)-pyrrolidinyl]glycyl]pyrrolidin-2(R)-ylboronic acid, and pharmaceutically acceptable salts, solvates, and hydrates thereof:

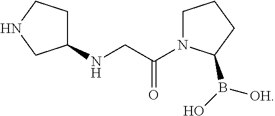 [SMILES: O=C(CN[C@@H]1CCNC1)N1CCC[C@H]1B(O)O]

The crystalline form of 1-[N-[3(R)-pyrrolidinyl]glycyl]pyrrolidin-2(R)-ylboronic acid tartrate is disclosed in international patent publication WO2008/027273. In some embodiments, the DPP-IV inhibitor is 1-[N-[3(R)-pyrrolidinyl]glycyl]pyrrolidin-2(R)-ylboronic acid tartrate:

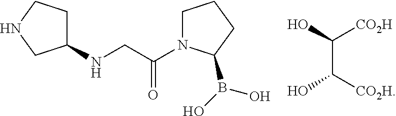 [SMILES: O=C(CN[C@@H]1CCNC1)N1CCC[C@H]1B(O)O ; O=C(O)[C@H](O)[C@@H](O)C(=O)O]

Melogliptin (GRC-8200, 4(S)-fluoro-1-[2-[(1R,3S)-3-(1H-1,2,4-triazol-1-ylmethyl)cyclopentylamino]acetyl]pyrrolidine-2(S)-carbonitrile) is a DPP-IV inhibitor currently undergoing phase 2 clinical trials by Glenmark Pharmaceuticals and Merck KGaA for the treatment of type 2 diabetes. The compound, 4(S)-fluoro-1-[2-[(1R,3S)-3-(1H-1,2,4-triazol-1-ylmethyl)cyclopentylamino]acetyl]pyrrolidine-2(S)-carbonitrile, is disclosed in international patent publication WO2006/040625. Some embodiments of the present invention include every combination of one or more compounds selected from compounds disclosed in WO2006/040625 and pharmaceutically acceptable salts, solvates, and hydrates thereof. In some embodiments, the DPP-IV inhibitor is selected from 4(S)-fluoro-1-[2-[(1R,3S)-3-(1H-1,2,4-triazol-1-ylmethyl)cyclopentylamino]acetyl]pyrrolidine-2(S)-carbonitrile, and pharmaceutically acceptable salts, solvates, and hydrates thereof:

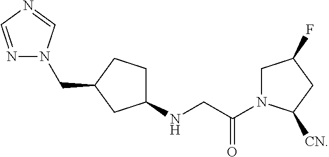 [SMILES: N#C[C@@H]1C[C@H](F)CN1C(=O)CN[C@@H]1CC[C@H](Cn2cncn2)C1]

Carmegliptin (R-1579, 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one) is a DPP-IV inhibitor. The compound, 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one, is disclosed in international patent publication WO2005/000848. Some embodiments of the present invention include every combination of one or more compounds selected from compounds disclosed in WO2005/000848 and pharmaceutically acceptable salts, solvates, and hydrates thereof. In some embodiments, the DPP-IV inhibitor is selected from 1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one, and pharmaceutically acceptable salts, solvates, and hydrates thereof:

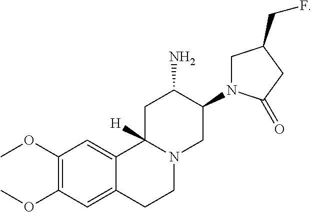 [SMILES: COc1cc2c(cc1OC)[C@@H]1C[C@H](N)[C@@H](N3C[C@@H](CF)CC3=O)CN1CC2]

Taisho disclosed (2S,4S)-2-cyano-4-fluoro-1-[(2-hydroxy-1,1-dimethyl) ethylamino]acetylpyrrolidine, a DPP-IV inhibitor in US patent publication US2007/0112059. Some embodiments of the present invention include every combination of one or more compounds selected from compounds disclosed in US2007/0112059 and pharmaceutically acceptable salts, solvates, and hydrates thereof. In some embodiments, the DPP-IV inhibitor is selected from (2S,4S)-2-cyano-4-fluoro-1-[(2-hydroxy-1,1-dimethyl)ethylamino]acetylpyrrolidine, and pharmaceutically acceptable salts, solvates, and hydrates thereof:

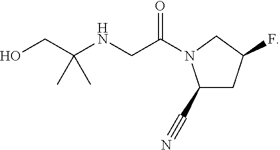 [SMILES: CC(C)(CO)NCC(=O)N1C[C@@H](F)C[C@H]1C#N]

Sanofi-Aventis disclosed a series of substituted bicyclic 8-pyrrolidineoxanthine derivatives as DPP-IV inhibitors in US publication US2007/0167468. Some embodiments of the present invention include every combination of one or more compounds selected from compounds disclosed in US publication US2007/0167468 and pharmaceutically acceptable salts, solvates, and hydrates thereof. In some embodiments, the DPP-IV inhibitor is selected from 8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione, and pharmaceutically acceptable salts, solvates, and hydrates thereof:

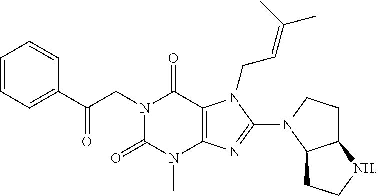 [SMILES: CC(C)=CCn1c(N2CC[C@H]3NCC[C@H]32)nc2c1c(=O)n(CC(=O)c1ccccc1)c(=O)n2C]

Pfizer disclosed a series of 3-amino-pyrrolidine-4-lactam derivatives as DPP-IV inhibitors in international patent publication WO2007/148185. Some embodiments of the present invention include every combination of one or more compounds selected from compounds disclosed in WO2007/148185 and pharmaceutically acceptable salts, solvates, and hydrates thereof. One such compound is 1-((3S,4S)-4-amino-1-(4-(3,3-difluoropyrrolidin-1-yl)-1,3,5-triazin-2-yl)pyrrolidin-3-yl)-5,5difluoropiperidin-2-one. In some embodiments, the DPP-IV inhibitor is selected from 1-((3S,4S)-4-amino-1-(4-(3,3-difluoropyrrolidin-1-yl)-1,3,5-triazin-2-yl)pyrrolidin-3-yl)-5,5difluoropiperidin-2-one, and pharmaceutically acceptable salts, solvates, and hydrates thereof:

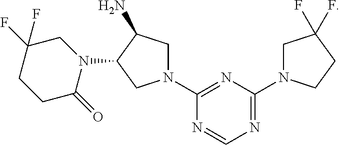 [SMILES: N[C@H]1CN(c2ncnc(N3CCC(F)(F)C3)n2)C[C@@H]1N1CC(F)(F)CCC1=O]

Syrrx disclosed a series of substituted pyrimidine-2,4(1H,3H)-dione derivatives as DPP-IV inhibitors in international patent publication WO2005/095381. Some embodiments of the present invention include every combination of one or more compounds selected from compounds disclosed in WO2005/095381 and pharmaceutically acceptable salts, solvates, and hydrates thereof. One such compound is (R)-2-(6-(3-aminopiperidin-1-yl)-3-methyl-2,4-dioxo-3,4-dihydropyrimidin-1(2H)-yl)methyl)-4-fluorobenzonitrile. In some embodiments, the DPP-IV inhibitor is selected from (R)-2-((6-(3-aminopiperidin-1-yl)-3-methyl-2,4-dioxo-3,4-dihydropyrimidin-1(2H)-yl)methyl)-4-fluorobenzonitrile, and pharmaceutically acceptable salts, solvates, and hydrates thereof:

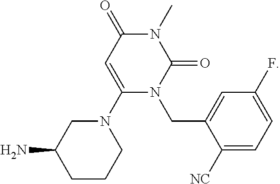 [SMILES: Cn1c(=O)cc(N2CCC[C@@H](N)C2)n(Cc2cc(F)ccc2C#N)c1=O]

Various crystalline forms of (R)-2-((6-(3-aminopiperidin-1-yl)-3-methyl-2,4-dioxo-3,4-dihydropyrimidin-1(2H)-yl)methyl)-4-fluorobenzonitrile succinic acid salt are disclosed in international patent publication WO2008/067465. One embodiment of the present invention pertains to any one or more crystalline forms of (R)-2-(6-(3-aminopiperidin-1-yl)-3-methyl-2,4-dioxo-3,4-dihydropyrimidin-1(2H)-yl)methyl)-4-fluorobenzonitrile succinic acid salt as described in international patent publication WO2008/067465. In some embodiments, the DPP-IV inhibitor is crystalline (R)-2-((6-(3-aminopiperidin-1-yl)-3-methyl-2,4-dioxo-3,4-dihydropyrimidin-1(2H)-yl)methyl)-4-fluorobenzonitrile succinic acid salt:

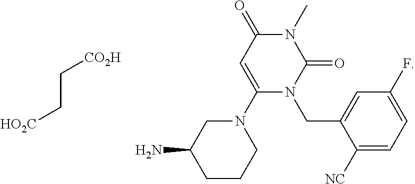 [SMILES: O=C(O)CCC(=O)O ; Cn1c(=O)cc(N2CCC[C@@H](N)C2)n(Cc2cc(F)ccc2C#N)c1=O]

Alantos disclosed a series of substituted 2-cyano-pyrrolidine derivatives as DPP-IV inhibitors in international patent publication WO2006/116157. Some embodiments of the present invention include every combination of one or more compounds selected from compounds disclosed in WO2006/116157 and pharmaceutically acceptable salts, solvates, and hydrates thereof. One such compound is 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide. In some embodiments, the DPP-IV inhibitor is selected from 5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide, and pharmaceutically acceptable salts, solvates, and hydrates thereof:

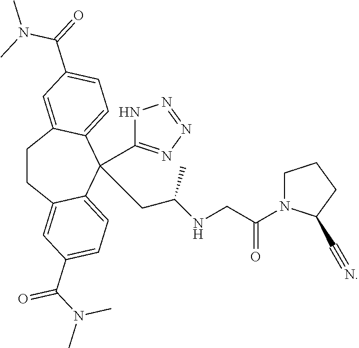 [SMILES: C[C@@H](CC1(c2nnn[nH]2)c2ccc(C(=O)N(C)C)cc2CCc2cc(C(=O)N(C)C)ccc21)NCC(=O)N1CCC[C@H]1C#N]

Mitsubishi disclosed a series of 2,4-disubstituted pyrrolidine derivatives as DPP-IV inhibitors in international patent publication WO2002/0014271. Some embodiments of the present invention include every combination of one or more compounds selected from compounds disclosed in WO2002/0014271 and pharmaceutically acceptable salts, solvates, and hydrates thereof. One such compound is ((2S,4S)-4-(4-(3-methyl-1-phenyl-1H-pyrazol-5-yl)piperazin-1-yl)pyrrolidin-2-yl)(thiazolidin-3-yl)methanone. In some embodiments, the DPP-IV inhibitor is selected from ((2S,4S)-4-(4-(3-methyl-1-phenyl-1H-pyrazol-5-yl)piperazin-1-yl)pyrrolidin-2-yl)(thiazolidin-3-yl)methanone, and pharmaceutically acceptable salts, solvates, and hydrates thereof:

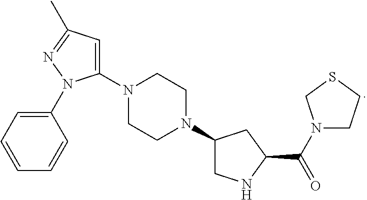 [SMILES: Cc1cc(N2CCN([C@@H]3CN[C@H](C(=O)N4CCSC4)C3)CC2)n(-c2ccccc2)n1]

Various crystalline forms of ((2S,4S)-4-(4-(3-methyl-1-phenyl-1H-pyrazol-5-yl)piperazin-1-yl)pyrrolidin-2-yl)(thiazolidin-3-yl)methanone salts are disclosed in international patent publication WO2006/088129 and US publication 2009/0216016. One embodiment of the present invention pertains to any one or more crystalline forms of ((2S,4S)-4-(4-(3-methyl-1-phenyl-1H-pyrazol-5-yl)piperazin-1-yl)pyrrolidin-2-yl)(thiazolidin-3-yl)methanone salt as described in international patent publication WO2006/088129 and US publication 2009/0216016. In some embodiments, the DPP-IV inhibitor is crystalline ((2S,4S)-4-(4-(3-methyl-1-phenyl-1H-pyrazol-5-yl)piperazin-1-yl)pyrrolidin-2-yl)(thiazolidin-3-yl)methanone 2.5 hydrobromide salt:

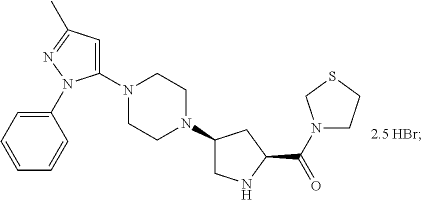 [SMILES: Cc1cc(N2CCN([C@@H]3CN[C@H](C(=O)N4CCSC4)C3)CC2)n(-c2ccccc2)n1]

or a mono or a dihydrate thereof. In some embodiments, the DPP-IV inhibitor is crystalline ((2S,4S)-4-(4-(3-methyl-1-phenyl-1H-pyrazol-5-yl)piperazin-1-yl)pyrrolidin-2-yl)(thiazolidin-3-yl)methanone di-hydrobromide salt.

Kyorin disclosed a series of pyrrolidinecarbonitrile derivatives as DPP-IV inhibitors in international patent publication WO2008/114857 and US publication US2008/0146818. Some embodiments of the present invention include every combination of one or more compounds selected from compounds disclosed in WO2008/114857 and US publication US2008/0146818, and pharmaceutically acceptable salts, solvates, and hydrates thereof. One such compound is (2S,4S)-1-[2-[(4-ethoxycarbonylbicyclo[2.2.2]oct-1-yl)amino]acetyl]-4-fluoropyrrolidine-2-carbonitrile. In some embodiments, the DPP-IV inhibitor is selected from (2S,4S)-1-[2-[(4-ethoxycarbonylbicyclo[2.2.2]oct-1-yl)amino]acetyl]-4-fluoropyrrolidine-2-carbonitrile, and pharmaceutically acceptable salts, solvates, and hydrates thereof:

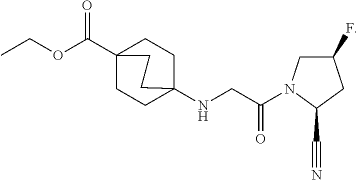 [SMILES: CCOC(=O)C12CCC(NCC(=O)N3C[C@@H](F)C[C@H]3C#N)(CC1)CC2]

Dainippon Sumitomo disclosed a series of bicyclic pyrrole derivatives as DPP-IV inhibitors in international patent publication WO2006/068163 and US publication US2009/0192129. Some embodiments of the present invention include every combination of one or more compounds selected from compounds disclosed in WO2006/068163 and US publication US2009/0192129 and pharmaceutically acceptable salts, solvates, and hydrates thereof. One such compound is (6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione. In some embodiments, the DPP-IV inhibitor is selected from (6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione, and pharmaceutically acceptable salts, solvates, and hydrates thereof:

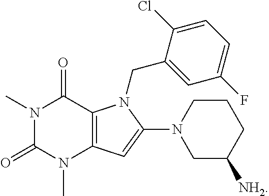 [SMILES: Cn1c(=O)c2c(cc(N3CCC[C@@H](N)C3)n2Cc2cc(F)ccc2Cl)n(C)c1=O]

Dainippon Sumitomo disclosed 2-([6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl]methyl)-4-fluorobenzonitrile as a DPP-IV inhibitor in international patent publication WO2009/084497. In some embodiments, the DPP-IV inhibitor is selected from 2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile, and pharmaceutically acceptable salts, solvates, and hydrates thereof:

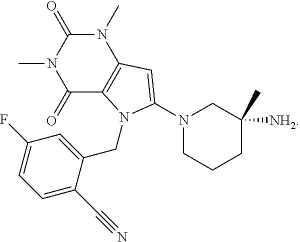 [SMILES: Cn1c(=O)c2c(cc(N3CCC[C@@](C)(N)C3)n2Cc2cc(F)ccc2C#N)n(C)c1=O]

Hoffmann-La Roche disclosed a series of N-substituted pyrrolidine derivatives as DPP-IV inhibitors in international patent publication WO03/037327. Some embodiments of the present invention include every combination of one or more compounds selected from compounds disclosed in WO03/037327 and pharmaceutically acceptable salts, solvates, and hydrates thereof. One such compound is (2S)-1-{[2-(5-methyl-2-phenyl-oxazol-4-yl)-ethylamino]-acetyl}-pyrrolidine-2-carbonitrile. In some embodiments, the DPP-IV inhibitor is selected from (2S)-1-{[2-(5-methyl-2-phenyl-oxazol-4-yl)-ethylamino]-acetyl}-pyrrolidine-2-carbonitrile, and pharmaceutically acceptable salts, solvates, and hydrates thereof:

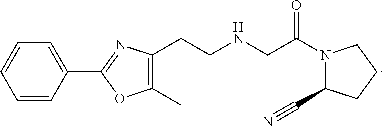 [SMILES: Cc1oc(-c2ccccc2)nc1CCNCC(=O)N1CCC[C@H]1C#N]

Various crystalline forms of (2S)-1-{[2-(5-methyl-2-phenyl-oxazol-4-yl)-ethylamino]-acetyl}-pyrrolidine-2-carbonitrile methanesulfonic acid salt are disclosed in international patent publication WO2006/100181. In some embodiments, the DPP-IV inhibitor is (2S)-1-{[2-(5-methyl-2-phenyl-oxazol-4-yl)-ethylamino]-acetyl}-pyrrolidine-2-carbonitrile methanesulfonic acid salt (i.e., mesylate):

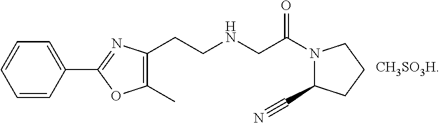 [SMILES: Cc1oc(-c2ccccc2)nc1CCNCC(=O)N1CCC[C@H]1C#N]

Other compounds disclosed by Hoffmann-La Roche in international patent publication WO03/037327 include (2S)-1-{[1,1-dimethyl-3-(4-pyridin-3-yl-imidazol-1-yl)-propylamino]-acetyl}-pyrrolidine-2-carbonitrile, and pharmaceutically acceptable salts thereof, such as the methanesulfonic acid salt. In some embodiments, the DPP-IV inhibitor is selected from (2S)-1-{[1,1-dimethyl-3-(4-pyridin-3-yl-imidazol-1-yl)-propylamino]-acetyl}-pyrrolidine-2-carbonitrile, and pharmaceutically acceptable salts, solvates, and hydrates thereof:

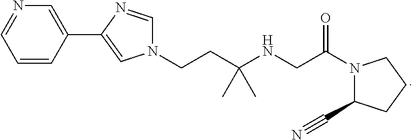 [SMILES: CC(C)(CCn1cnc(-c2cccnc2)c1)NCC(=O)N1CCC[C@H]1C#N]

In some embodiments, the DPP-IV inhibitor is (2S)-1-{[1,1-dimethyl-3-(4-pyridin-3-yl-imidazol-1-yl)-propylamino]-acetyl}-pyrrolidine-2-carbonitrile methanesulfonic acid:

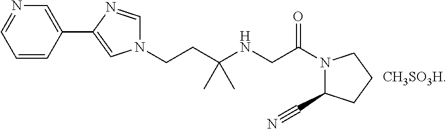 [SMILES: CC(C)(CCn1cnc(-c2cccnc2)c1)NCC(=O)N1CCC[C@H]1C#N]

Various crystalline forms of (2S)-1-{[1,1-dimethyl-3-(4-pyridin-3-yl-imidazol-1-yl)-propylamino}-acetyl]-pyrrolidine-2-carbonitrile fumaric acid salt are disclosed in international patent publication WO2007/071576. In some embodiments, the DPP-IV inhibitor is (2S)-1-{[1,1-dimethyl-3-(4-pyridin-3-yl-imidazol-1-yl)-propylamino]-acetyl}-pyrrolidine-2-carbonitrile fumaric acid salt (i.e., fumarate):

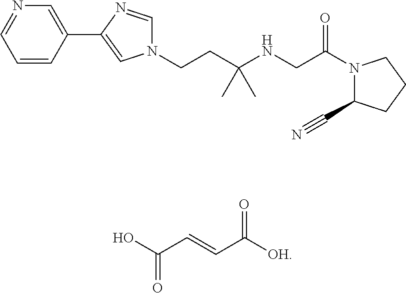 [SMILES: CC(C)(CCn1cnc(-c2cccnc2)c1)NCC(=O)N1CCC[C@H]1C#N ; O=C(O)/C=C/C(=O)O]

Pfizer disclosed a series of proline derivatives as DPP-IV inhibitors in international patent publication WO2005/116014. Some embodiments of the present invention include every combination of one or more compounds selected from compounds disclosed in WO2005/116014 and pharmaceutically acceptable salts, solvates, and hydrates thereof. One such compound is (3,3-difluoropyrrolidin-1-yl)-((2S,4S)-4-(4-(pyrimidin-2-yl)piperazin-1-yl)pyrrolidin-2-yl)methanone. In some embodiments, the DPP-IV inhibitor is selected from (3,3-difluoropyrrolidin-1-yl)-((2S,4S)-4-(4-(pyrimidin-2-yl)piperazin-1-yl)pyrrolidin-2-yl)methanone, and pharmaceutically acceptable salts, solvates, and hydrates thereof:

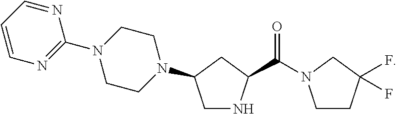 [SMILES: O=C([C@@H]1C[C@H](N2CCN(c3ncccn3)CC2)CN1)N1CCC(F)(F)C1]

GlaxoSmithKline disclosed a series of fluoropyrrolidine derivatives as DPP-IV inhibitors in international patent publication WO03/002531. Some embodiments of the present invention include every combination of one or more compounds selected from the DPP-IV inhibitors disclosed in WO03/037327 and pharmaceutically acceptable salts, solvates, and hydrates thereof. One such compound is (2S,4S)-1-[(2S)-2-amino-3,3-bis(4-fluorophenyl)propanoyl]-4-fluoropyrrolidine-2-carbonitrile (Denagliptin). In some embodiments, the DPP-IV inhibitor is selected from (2S,4S)-1-[(2S)-2-amino-3,3-bis(4-fluorophenyl)propanoyl]-4-fluoropyrrolidine-2-carbonitrile, and pharmaceutically acceptable salts, solvates, and hydrates thereof:

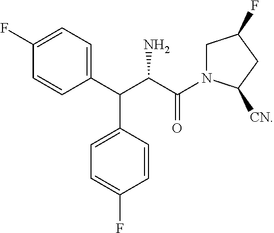 [SMILES: N#C[C@@H]1C[C@H](F)CN1C(=O)[C@@H](N)C(c1ccc(F)cc1)c1ccc(F)cc1]

Various crystalline forms of (2S,4S)-1-[(2S)-2-amino-3,3-bis(4-fluorophenyl)propanoyl]-4-fluoropyrrolidine-2-carbonitrile and salts have been disclosed in international patent publication WO2005/009956. One salt disclosed is (2S,4S)-1-[(2S)-2-amino-3,3-bis(4-fluorophenyl)propanoyl]-4-fluoropyrrolidine-2-carbonitrile p-toluenesulfonic acid salt (also referred to as (2S,4S)-4-fluoro-1-[4-fluoro-β-(4-fluorophenyl)-L-phenylalanyl]-2-pyrrolidinecarbonitrile p-toluenesulfonic acid salt, or Denagliptin tosylate). In some embodiments, the DPP-IV inhibitor is (2S,4S)-1-[(2S)-2-amino-3,3-bis(4-fluorophenyl)propanoyl]-4-fluoropyrrolidine-2-carbonitrile p-toluenesulfonic acid salt:

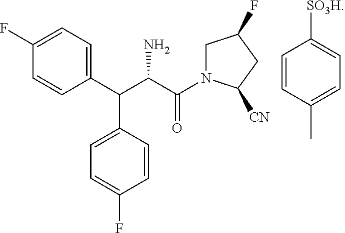

Abbott disclosed a series of substituted pyrrolidinyl derivatives as DPP-IV inhibitors in international patent publication WO2004/026822. Some embodiments of the present invention include every combination of one or more compounds selected from the DPP-IV inhibitors disclosed in WO2004/026822 and pharmaceutically acceptable salts, solvates, and hydrates thereof. One such compound is (2S,5R)-5-ethynyl-1-{N-(4-methyl-1-(4-carboxy-pyridin-2-yl)piperidin-4-yl)glycyl}pyrrolidine-2-carbonitrile. In some embodiments, the DPP-IV inhibitor is selected from (2S,5R)-5-ethynyl-1-{N-(4-methyl-1-(4-carboxy-pyridin-2-yl)piperidin-4-yl)glycyl}pyrrolidine-2-carbonitrile, and pharmaceutically acceptable salts, solvates, and hydrates thereof:

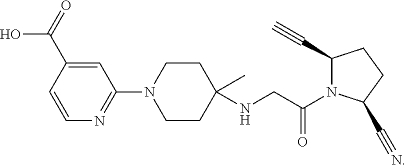 [SMILES: C#C[C@H]1CC[C@@H](C#N)N1C(=O)CNC1(C)CCN(c2cc(C(=O)O)ccn2)CC1]

Abbott has further disclosed a series of substituted cyclohexanyl/cyclohexenyl derivatives as DPP-IV inhibitors in international patent publication WO2007/027651. Some embodiments of the present invention include every combination of one or more compounds selected from the DPP-IV inhibitors disclosed in WO2007/027651 and pharmaceutically acceptable salts, solvates, and hydrates thereof. One such compound is (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine. In some embodiments, the DPP-IV inhibitor is selected from (1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine, and pharmaceutically acceptable salts, solvates, and hydrates thereof:

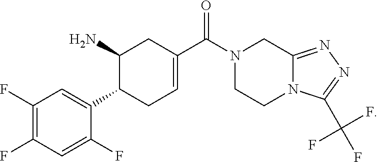 [SMILES: N[C@H]1CC(C(=O)N2CCn3c(nnc3C(F)(F)F)C2)=CC[C@@H]1c1cc(F)c(F)cc1F]

### Biguanides

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin ((phenylethyl)biguanide), metformin (dimethylbiguanide), buformin (butylbiguanide), proguanil (1-(p-chlorophenyl)-5-isopropylbiguanide), and biguanides known in the art.

In some embodiments, the first pharmaceutical agent is a biguanide selected from (phenylethyl)biguanide (chemical structure shown below) and pharmaceutically acceptable salts, solvates, and hydrates thereof:

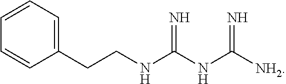 [SMILES: N=C(N)NC(=N)NCCc1ccccc1]

In some embodiments, the first pharmaceutical agent is a biguanide selected from dimethylbiguanide (chemical structure shown below) and pharmaceutically acceptable salts, solvates, and hydrates thereof; the chemical structure is as follows:

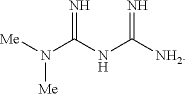

In some embodiments, the first pharmaceutical agent is a biguanide selected from butylbiguanide (chemical structure shown below) and pharmaceutically acceptable salts, solvates, and hydrates thereof; the chemical structure is as follows:

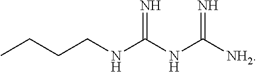

In some embodiments, the first pharmaceutical agent is a biguanide selected from 1-(p-chlorophenyl)-5-isopropylbiguanide (chemical structure shown below) and pharmaceutically acceptable salts, solvates, and hydrates thereof; the chemical structure is as follows:

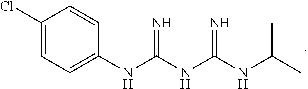 [SMILES: CC(C)NC(=N)NC(=N)Nc1ccc(Cl)cc1]

In some embodiments, the first pharmaceutical agent is a biguanide selected from the following biguanides: metformin, phenformin, buformin, and proguanil. In some embodiments, the first pharmaceutical agent is metformin. In some embodiments, the first pharmaceutical agent is phenformin. In some embodiments, the first pharmaceutical agent is buformin. In some embodiments, the first pharmaceutical agent is proguanil.

In some embodiments, the second pharmaceutical agent is a biguanide selected from (phenylethyl)biguanide (chemical structure shown below) and pharmaceutically acceptable salts, solvates, and hydrates thereof:

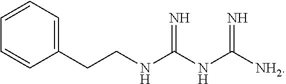 [SMILES: N=C(N)NC(=N)NCCc1ccccc1]

In some embodiments, the second pharmaceutical agent is a biguanide selected from dimethylbiguanide (chemical structure shown below) and pharmaceutically acceptable salts, solvates, and hydrates thereof; the chemical structure is as follows:

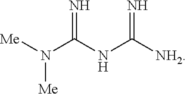

In some embodiments, the second pharmaceutical agent is a biguanide selected from butylbiguanide (chemical structure shown below) and pharmaceutically acceptable salts, solvates, and hydrates thereof; the chemical structure is as follows:

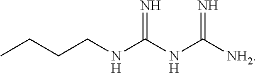

In some embodiments, the second pharmaceutical agent is a biguanide selected from 1-(p-chlorophenyl)-5-isopropylbiguanide (chemical structure shown below) and pharmaceutically acceptable salts, solvates, and hydrates thereof; the chemical structure is as follows:

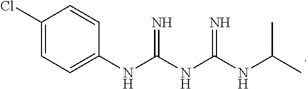 [SMILES: CC(C)NC(=N)NC(=N)Nc1ccc(Cl)cc1]

In some embodiments, the second pharmaceutical agent is a biguanide selected from the following biguanides: metformin, phenformin, buformin, and proguanil. In some embodiments, the second pharmaceutical agent is metformin. In some embodiments, the second pharmaceutical agent is phenformin. In some embodiments, the second pharmaceutical agent is buformin. In some embodiments, the second pharmaceutical agent is proguanil.

### Alpha-Glucosidase Inhibitors

Alpha-Glucosidase inhibitors belong to the class of drugs which competitively inhibit digestive enzymes such as alpha-amylase, maltase, alpha-dextrinase, sucrase, etc. in the pancreas and or small intestine. The reversible inhibition by alpha-glucosidase inhibitors retard, diminish or otherwise reduce blood glucose levels by delaying the digestion of starch and sugars. Some representative examples of α-glucosidase inhibitors include acarbose ((2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal), miglitol ((2R,3R,4R,5S)-1-(2-hydroxyethyl)-2-(hydroxymethyl)piperidine-3,4,5-triol), voglibose ((1S,2S,3R,4S,5S)-5-(1,3-dihydroxypropan-2-ylamino)-1-(hydroxymethyl)cyclohexane-1,2,3,4-tetraol), and α-glucosidase inhibitors known in the art.

In some embodiments, the first pharmaceutical agent is a α-glucosidase inhibitor selected from (2R,3R,4R,5R)-4-(2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal (chemical structure shown below) and pharmaceutically acceptable salts, solvates, and hydrates thereof:

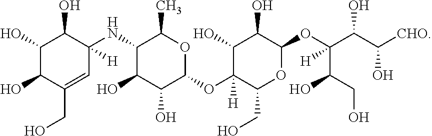 [SMILES: C[C@H]1O[C@H](O[C@H]2[C@H](O)[C@@H](O)[C@@H](O[C@@H]([C@H](O)[C@@H](O)C=O)[C@H](O)CO)O[C@@H]2CO)[C@H](O)[C@@H](O)[C@@H]1N[C@H]1C=C(CO)[C@@H](O)[C@H](O)[C@H]1O]

In some embodiments, the first pharmaceutical agent is a α-glucosidase inhibitor selected from (2R,3R,4R,5S)-1-(2-hydroxyethyl)-2-(hydroxymethyl)piperidine-3,4,5-triol (chemical structure shown below) and pharmaceutically acceptable salts, solvates, and hydrates thereof:

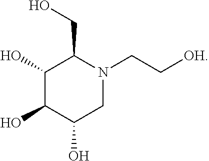 [SMILES: OCCN1C[C@H](O)[C@@H](O)[C@H](O)[C@H]1CO]

In some embodiments, the first pharmaceutical agent is a α-glucosidase inhibitor selected from (1S,2S,3R,4S,5S)-5-(1,3-dihydroxypropan-2-ylamino)-1-(hydroxymethyl)cyclohexane-1,2,3,4-tetraol (chemical structure shown below) and pharmaceutically acceptable salts, solvates, and hydrates thereof:

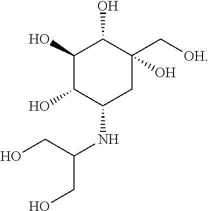 [SMILES: OCC(CO)N[C@H]1C[C@](O)(CO)[C@@H](O)[C@H](O)[C@H]1O]

In some embodiments, the first pharmaceutical agent is an alpha-glucosidase inhibitor selected from: acarbose, miglitol, and voglibose. In some embodiments, the pharmaceutical agent or the first pharmaceutical agent is acarbose. In some embodiments, the pharmaceutical agent or the first pharmaceutical agent is miglitol. In some embodiments, the pharmaceutical agent or the first pharmaceutical agent is voglibose.

In some embodiments, the first pharmaceutical agent is a α-glucosidase inhibitor selected from (2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal (chemical structure shown below) and pharmaceutically acceptable salts, solvates, and hydrates thereof:

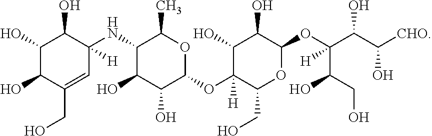 [SMILES: C[C@H]1O[C@H](O[C@H]2[C@H](O)[C@@H](O)[C@@H](O[C@@H]([C@H](O)[C@@H](O)C=O)[C@H](O)CO)O[C@@H]2CO)[C@H](O)[C@@H](O)[C@@H]1N[C@H]1C=C(CO)[C@@H](O)[C@H](O)[C@H]1O]

In some embodiments, the first pharmaceutical agent is a α-glucosidase inhibitor selected from (2R,3R,4R,5S)-1-(2-hydroxyethyl)-2-(hydroxymethyl)piperidine-3,4,5-triol (chemical structure shown below) and pharmaceutically acceptable salts, solvates, and hydrates thereof:

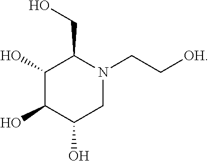 [SMILES: OCCN1C[C@H](O)[C@@H](O)[C@H](O)[C@H]1CO]

In some embodiments, the first pharmaceutical agent is a α-glucosidase inhibitor selected from (1S,2S,3R,4S,5S)-5-(1,3-dihydroxypropan-2-ylamino)-1-(hydroxymethyl)cyclohexane-1,2,3,4-tetraol (chemical structure shown below) and pharmaceutically acceptable salts, solvates, and hydrates thereof:

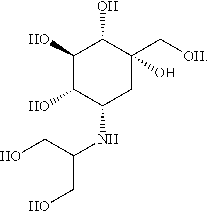 [SMILES: OCC(CO)N[C@H]1C[C@](O)(CO)[C@@H](O)[C@H](O)[C@H]1O]

In some embodiments, the first pharmaceutical agent is an alpha-glucosidase inhibitor selected from: acarbose, miglitol, and voglibose. In some embodiments, the pharmaceutical agent or the first pharmaceutical agent is acarbose. In some embodiments, the pharmaceutical agent or the first pharmaceutical agent is miglitol. In some embodiments, the pharmaceutical agent or the first pharmaceutical agent is voglibose.

In some embodiments, the first pharmaceutical agent is a α-glucosidase inhibitor selected from (2R,3R,4R,5R)-4-((2R,3R,4R,5S,6R)-5-((2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal (chemical structure shown below) and pharmaceutically acceptable salts, solvates, and hydrates thereof:

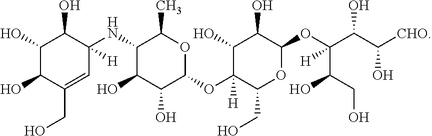 [SMILES: C[C@H]1O[C@H](O[C@H]2[C@H](O)[C@@H](O)[C@@H](O[C@@H]([C@H](O)[C@@H](O)C=O)[C@H](O)CO)O[C@@H]2CO)[C@H](O)[C@@H](O)[C@@H]1N[C@H]1C=C(CO)[C@@H](O)[C@H](O)[C@H]1O]

In some embodiments, the first pharmaceutical agent is a α-glucosidase inhibitor selected from (2R,3R,4R,5S)-1-(2-hydroxyethyl)-2-(hydroxymethyl)piperidine-3,4,5-triol (chemical structure shown below) and pharmaceutically acceptable salts, solvates, and hydrates thereof:

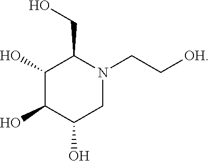 [SMILES: OCCN1C[C@H](O)[C@@H](O)[C@H](O)[C@H]1CO]

In some embodiments, the first pharmaceutical agent is a α-glucosidase inhibitor selected from (1S,2S,3R,4S,5S)-5-(1,3-dihydroxypropan-2-ylamino)-1-(hydroxymethyl)cyclohexane-1,2,3,4-tetraol (chemical structure shown below) and pharmaceutically acceptable salts, solvates, and hydrates thereof:

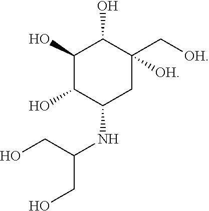 [SMILES: OCC(CO)N[C@H]1C[C@](O)(CO)[C@@H](O)[C@H](O)[C@H]1O]

In some embodiments, the first pharmaceutical agent is an alpha-glucosidase inhibitor selected from: acarbose, miglitol, and voglibose. In some embodiments, the pharmaceutical agent or the first pharmaceutical agent is acarbose. In some embodiments, the pharmaceutical agent or the first pharmaceutical agent is miglitol. In some embodiments, the pharmaceutical agent or the first pharmaceutical agent is voglibose.

In some embodiments, the second pharmaceutical agent is a α-glucosidase inhibitor selected from (2R,3R,4R,5R)-4-(2R,3R,4R,5S,6R)-5-(2R,3R,4S,5S,6R)-3,4-dihydroxy-6-methyl-5-((1S,4R,5S,6S)-4,5,6-trihydroxy-3-(hydroxymethyl)cyclohex-2-enylamino)tetrahydro-2H-pyran-2-yloxy)-3,4-dihydroxy-6-(hydroxymethyl)tetrahydro-2H-pyran-2-yloxy)-2,3,5,6-tetrahydroxyhexanal (chemical structure shown below) and pharmaceutically acceptable salts, solvates, and hydrates thereof:

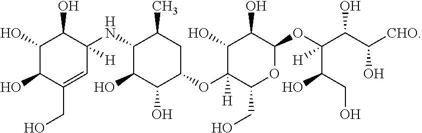 [SMILES: C[C@H]1C[C@H](O[C@H]2[C@H](O)[C@@H](O)[C@@H](O[C@@H]([C@H](O)[C@@H](O)C=O)[C@H](O)CO)O[C@@H]2CO)[C@H](O)[C@@H](O)[C@@H]1N[C@H]1C=C(CO)[C@@H](O)[C@H](O)[C@H]1O]

In some embodiments, the second pharmaceutical agent is a α-glucosidase inhibitor selected from (2R,3R,4R,5S)-1-(2-hydroxyethyl)-2-(hydroxymethyl)piperidine-3,4,5-triol (chemical structure shown below) and pharmaceutically acceptable salts, solvates, and hydrates thereof:

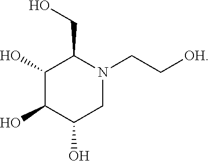 [SMILES: OCCN1C[C@H](O)[C@@H](O)[C@H](O)[C@H]1CO]

In some embodiments, the second pharmaceutical agent is a α-glucosidase inhibitor selected from (1S,2S,3R,4S,5S)-5-(1,3-dihydroxypropan-2-ylamino)-1-(hydroxymethyl)cyclohexane-1,2,3,4-tetraol (chemical structure shown below) and pharmaceutically acceptable salts, solvates, and hydrates thereof:

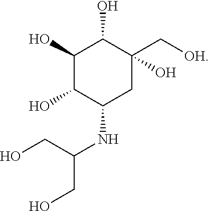 [SMILES: OCC(CO)N[C@H]1C[C@](O)(CO)[C@@H](O)[C@H](O)[C@H]1O]

In some embodiments, the second pharmaceutical agent is an alpha-glucosidase inhibitor selected from: acarbose, miglitol, and voglibose. In some embodiments, the pharmaceutical agent or the second pharmaceutical agent is acarbose. In some embodiments, the pharmaceutical agent or the second pharmaceutical agent is miglitol. In some embodiments, the pharmaceutical agent or the second pharmaceutical agent is voglibose.

### Insulin and Insulin Analogues

The term “insulin analogue” refers to the naturally occurring human hormone and insulin receptor ligands (i.e., synthetic insulin analogues). Insulin receptor ligands are structurally different from the natural human hormone, but have substantially the same activity as human insulin in terms of glycemic control. Examples of an insulin analogue include, NPH insulin (also known as Humulin N, Novolin N, NPH Lletin II, and insulin isophane), insulin lispro (28B-L-lysine-29B-L-proline-insulin, wherein insulin is human insulin), insulin aspart (28B-L-aspartic acid-insulin, wherein insulin is human insulin), insulin glulisine (3B-L-lysine-29B-L-glutamic acid-insulin, wherein insulin is human insulin), and insulin analogues known in the art.

NPH insulin is marketed by Eli Lilly and Company under the name Humulin N, and is considered as an intermediate-acting insulin analogue given to help control the blood sugar level of those with diabetes. Insulin lispro is marketed by Eli Lilly and Company under the name Humalog, and is considered a rapid acting insulin analogue. Insulin aspart is marketed by Novo Nordisk and sold as NovoRapid. Insulin aspart is considered a fast acting insulin analogue. Insulin glulisine was developed by Sanofi-Aventis and is sold under the trade name Apidra. Insulin glulisine is considered a rapid acting insulin analogue but shorter duration of action compared to human insulin.

In some embodiments, the first pharmaceutical agent is an insulin analogue selected from NPH insulin and pharmaceutically acceptable salts, solvates, and hydrates thereof. In some embodiments, the first pharmaceutical agent is an insulin analogue selected from insulin lispro and pharmaceutically acceptable salts, solvates, and hydrates thereof. In some embodiments, the first pharmaceutical agent is an insulin analogue selected from insulin aspart and pharmaceutically acceptable salts, solvates, and hydrates thereof. In some embodiments, the first pharmaceutical agent is an insulin analogue selected from insulin glulisine and pharmaceutically acceptable salts, solvates, and hydrates thereof.

In some embodiments, the second pharmaceutical agent is an insulin analogue selected from NPH insulin and pharmaceutically acceptable salts, solvates, and hydrates thereof. In some embodiments, the second pharmaceutical agent is an insulin analogue selected from insulin lispro and pharmaceutically acceptable salts, solvates, and hydrates thereof. In some embodiments, the second pharmaceutical agent is an insulin analogue selected from insulin aspart and pharmaceutically acceptable salts, solvates, and hydrates thereof. In some embodiments, the second pharmaceutical agent is an insulin analogue selected from insulin glulisine and pharmaceutically acceptable salts, solvates, and hydrates thereof.

### Sulfonylureas

The sulfonylureas are drugs which promote secretion of insulin from pancreatic beta-cells by transmitting signals of insulin secretion via receptors in the cell membranes. Examples of a sulfonylurea include tolbutamide (Orinase, N-(butylcarbamoyl)-4-methylbenzenesulfonamide); acetohexamide (Dymelor, 4-acetyl-N-(cyclohexylcarbamoyl)benzenesulfonamide); tolazamide (Tolinase, N-(azepan-1-ylcarbamoyl)-4-methylbenzenesulfonamide); chlorpropamide (Diabinese, 4-chloro-N-(propylcarbamoyl)benzenesulfonamide); glipizide (Glucotrol, N-(4-(N-(cyclohexylcarbamoyl)sulfamoyl)phenethyl)-5-methylpyrazine-2-carboxamide); glibenclamide, also known as glyburide (Diabeta, Micronase, Glynase, 5-chloro-N-(4-(N-(cyclohexylcarbamoyl)sulfamoyl)phenethyl)-2-methoxybenzamide); glimepiride (Amaryl, 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide); gliclazide (Diamicron, N-(hexahydrocyclopenta[c]pyrrol-2(1H)-ylcarbamoyl)-4-methylbenzenesulfonamide); and sulfonylureas known in the art.

In some embodiments, the first pharmaceutical agent is a sulfonylurea selected from N-(butylcarbamoyl)-4-methylbenzenesulfonamide (chemical structure shown below) and pharmaceutically acceptable salts, solvates, and hydrates thereof:

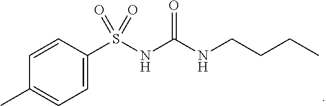 [SMILES: CCCCNC(=O)NS(=O)(=O)c1ccc(C)cc1]

In some embodiments, the first pharmaceutical agent is a sulfonylurea selected from 4-acetyl-N-(cyclohexylcarbamoyl)benzenesulfonamide (chemical structure shown below) and pharmaceutically acceptable salts, solvates, and hydrates thereof:

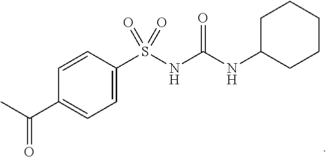 [SMILES: CC(=O)c1ccc(S(=O)(=O)NC(=O)NC2CCCCC2)cc1]

In some embodiments, the first pharmaceutical agent is a sulfonylurea selected from N-(azepan-1-ylcarbamoyl)-4-methylbenzenesulfonamide (chemical structure shown below) and pharmaceutically acceptable salts, solvates, and hydrates thereof:

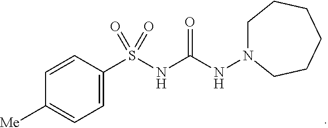 [SMILES: Cc1ccc(S(=O)(=O)NC(=O)NN2CCCCCC2)cc1]

In some embodiments, the first pharmaceutical agent is a sulfonylurea selected from 4-chloro-N-(propylcarbamoyl)benzenesulfonamide (chemical structure shown below) and pharmaceutically acceptable salts, solvates, and hydrates thereof:

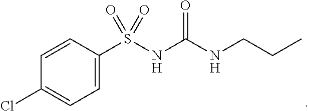 [SMILES: CCCNC(=O)NS(=O)(=O)c1ccc(Cl)cc1]

In some embodiments, the first pharmaceutical agent is a sulfonylurea selected from N-(4-(N-(cyclohexylcarbamoyl)sulfamoyl)phenethyl)-5-methylpyrazine-2-carboxamide (chemical structure shown below) and pharmaceutically acceptable salts, solvates, and hydrates thereof:

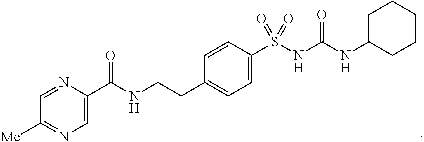 [SMILES: Cc1cnc(C(=O)NCCc2ccc(S(=O)(=O)NC(=O)NC3CCCCC3)cc2)cn1]

In some embodiments, the first pharmaceutical agent is a sulfonylurea selected from 5-chloro-N-(4-(N-(cyclohexylcarbamoyl)sulfamoyl)phenethyl)-2-methoxybenzamide (chemical structure shown below) and pharmaceutically acceptable salts, solvates, and hydrates thereof:

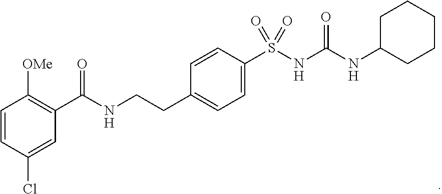 [SMILES: COc1ccc(Cl)cc1C(=O)NCCc1ccc(S(=O)(=O)NC(=O)NC2CCCCC2)cc1]

In some embodiments, the first pharmaceutical agent is a sulfonylurea selected from 3-ethyl-4-methyl-N-(4-(N-((1r,4r)-4-methylcyclohexylcarbamoyl)sulfamoyl)phenethyl)-2-oxo-2,5-dihydro-1H-pyrrole-1-carboxamide (chemical structure shown below) and pharmaceutically acceptable salts, solvates, and hydrates thereof:

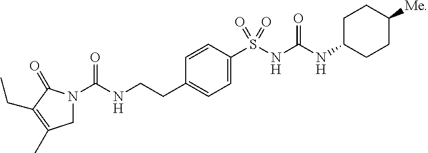 [SMILES: CCC1=C(C)CN(C(=O)NCCc2ccc(S(=O)(=O)NC(=O)N[C@H]3CC[C@H](C)CC3)cc2)C1=O]

In some embodiments, the first pharmaceutical agent is a sulfonylurea selected from N-(hexahydrocyclopenta[c]pyrrol-2(1H)-ylcarbamoyl)-4-methylbenzenesulfonamide (chemical structure shown below) and pharmaceutically acceptable salts, solvates, and hydrates thereof:

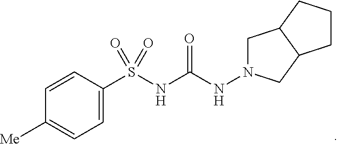 [SMILES: Cc1ccc(S(=O)(=O)NC(=O)NN2CC3CCCC3C2)cc1]

In some embodiments, the first pharmaceutical agent is a sulfonylurea selected from the following sulfonylureas and pharmaceutically acceptable salts, solvates, and hydrates thereof: glipizide, glimepiride, and glibenclamide. In some embodiments, the first pharmaceutical agent is tolbutamide. In some embodiments, the first pharmaceutical agent is acetohexamide. In some embodiments, the first pharmaceutical agent is tolazamide. In some embodiments, the first pharmaceutical agent is chlorpropamide. In some embodiments, the first pharmaceutical agent is glipizide. In some embodiments, the first pharmaceutical agent is glyburide. In some embodiments, the first pharmaceutical agent is glimepiride. In some embodiments, the first pharmaceutical agent is gliclazide.

In some embodiments, the second pharmaceutical agent is a sulfonylurea selected from N-(butylcarbamoyl)-4-methylbenzenesulfonamide (chemical structure shown below) and pharmaceutically acceptable salts, solvates, and hydrates thereof:

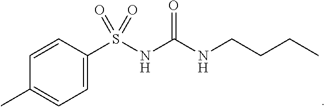 [SMILES: CCCCNC(=O)NS(=O)(=O)c1ccc(C)cc1]

In some embodiments, the second pharmaceutical agent is a sulfonylurea selected from 4-acetyl-N-(cyclohexylcarbamoyl)benzenesulfonamide (chemical structure shown below) and pharmaceutically acceptable salts, solvates, and hydrates thereof:

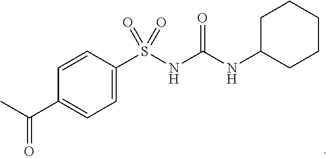 [SMILES: CC(=O)c1ccc(S(=O)(=O)NC(=O)NC2CCCCC2)cc1]

In some embodiments, the second pharmaceutical agent is a sulfonylurea selected from N-(azepan-1-ylcarbamoyl)-4-methylbenzenesulfonamide (chemical structure shown below) and pharmaceutically acceptable salts, solvates, and hydrates thereof:

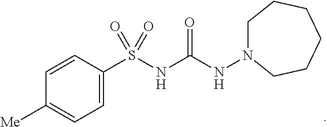 [SMILES: Cc1ccc(S(=O)(=O)NC(=O)NN2CCCCCC2)cc1]

In some embodiments, the second pharmaceutical agent is a sulfonylurea selected from 4-chloro-N-(propylcarbamoyl)benzenesulfonamide (chemical structure shown below) and pharmaceutically acceptable salts, solvates, and hydrates thereof:

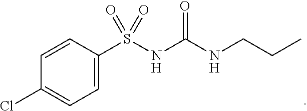 [SMILES: CCCNC(=O)NS(=O)(=O)c1ccc(Cl)cc1]

In some embodiments, the second pharmaceutical agent is a sulfonylurea selected from N-(4-(N-(cyclohexylcarbamoyl)sulfamoyl)phenethyl)-5-methylpyrazine-2-carboxamide (chemical structure shown below) and pharmaceutically acceptable salts, solvates, and hydrates thereof:

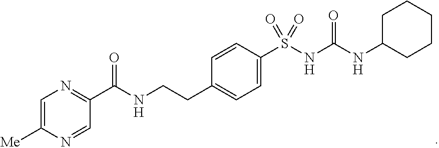 [SMILES: Cc1cnc(C(=O)NCCc2ccc(S(=O)(=O)NC(=O)NC3CCCCC3)cc2)cn1]

In some embodiments, the second pharmaceutical agent is a sulfonylurea selected from 5-chloro-N-(4-(N-(cyclohexylcarbamoyl)sulfamoyl)phenethyl)-2-methoxybenzamide (chemical structure shown below) and pharmaceutically acceptable salts, solvates, and hydrates thereof:

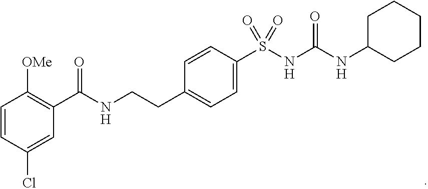 [SMILES: COc1ccc(Cl)cc1C(=O)NCCc1ccc(S(=O)(=O)NC(=O)NC2CCCCC2)cc1]

In some embodiments, the second pharmaceutical agent is a sulfonylurea selected from 3-ethyl-4-methyl-N-(4-(N-((1 r,4r)-4-methylcyclohexylcarbamoyl)sulfamoyl)phenethyl)-2-oxo-2,5-dihydro-1H-pyrrole-1-carboxamide (chemical structure shown below) and pharmaceutically acceptable salts, solvates, and hydrates thereof:

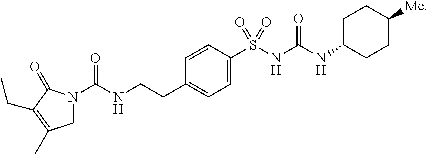 [SMILES: CCC1=C(C)CN(C(=O)NCCc2ccc(S(=O)(=O)NC(=O)N[C@H]3CC[C@H](C)CC3)cc2)C1=O]

In some embodiments, the second pharmaceutical agent is a sulfonylurea selected from N-(hexahydrocyclopenta[c]pyrrol-2(1H)-ylcarbamoyl)-4-methylbenzenesulfonamide (chemical structure shown below) and pharmaceutically acceptable salts, solvates, and hydrates thereof:

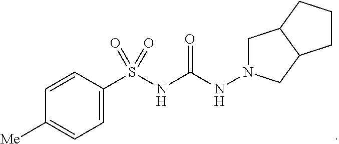 [SMILES: Cc1ccc(S(=O)(=O)NC(=O)NN2CC3CCCC3C2)cc1]

In some embodiments, the second pharmaceutical agent is a sulfonylurea selected from the following sulfonylureas and pharmaceutically acceptable salts, solvates, and hydrates thereof: glipizide, glimepiride, and glibenclamide. In some embodiments, the second pharmaceutical agent is tolbutamide. In some embodiments, the second pharmaceutical agent is acetohexamide. In some embodiments, the second pharmaceutical agent is tolazamide. In some embodiments, the second pharmaceutical agent is chlorpropamide. In some embodiments, the second pharmaceutical agent is glipizide. In some embodiments, the second pharmaceutical agent is glyburide. In some embodiments, the second pharmaceutical agent is glimepiride. In some embodiments, the second pharmaceutical agent is gliclazide.

### SGLT2 Inhibitors

Sodium-glucose transporter-2 (SGLT2) inhibitors belong to the class of drugs which inhibit the protein SGLT2 and the reabsorption of glucose in the kidney. The inhibition by SGLT2 inhibitors retard, diminish, or otherwise reduce the amount of glucose that is reabsorbed and therefore is eliminated in the urine. Some representative examples of SGLT2 inhibitors include dapagliflozin ((2S,3R,4R,5S,6R)-2-(4-chloro-3-(4-ethoxybenzyl)phenyl)-6-(hydroxymethyl)tetrahydro-2H-pyran-3,4,5-triol, Bristol-Myers Squibb and AstraZeneca), remogliflozin (ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate, GlaxoSmithKline), ASP1941 (Kotobuki/Astellas), canagliflozin ((2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol, Johnson & Johnson/Mitsubishi/Tanabe), ISIS 388626 (an antisense oligonucleotide, Isis Pharmaceuticals), sergliflozin (ethyl ((2R,3S,4S,5R,6S)-3,4,5-trihydroxy-6-(2-(4-methoxybenzyl)phenoxy)tetrahydro-2H-pyran-2-yl)methyl carbonate, GlaxoSmithKline), AVE2268 ((2R,3S,4S,5R,6S)-2-(hydroxymethyl)-6-(2-(4-methoxybenzyl)thiophen-3-yloxy)tetrahydro-2H-pyran-3,4,5-triol, Sanofi-Aventis), BI10773 (Boehringer Ingelheim), CSG453 (Chugai/Roche), LX4211 (Lexicon), and SGLT2 inhibitors known in the art.

In some embodiments, the first pharmaceutical agent is a sulfonylurea selected from (2S,3R,4R,5S,6R)-2-(4-chloro-3-(4-ethoxybenzyl)phenyl)-6-(hydroxymethyl)tetrahydro-2H-pyran-3,4,5-triol (chemical structure shown below) and pharmaceutically acceptable salts, solvates, and hydrates thereof:

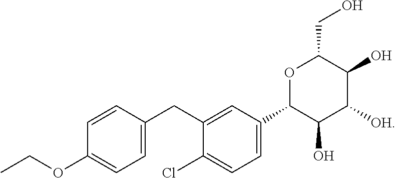 [SMILES: CCOc1ccc(Cc2cc([C@@H]3O[C@H](CO)[C@@H](O)[C@H](O)[C@H]3O)ccc2Cl)cc1]

In some embodiments, the first pharmaceutical agent is a sulfonylurea selected from ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate (chemical structure shown below) and pharmaceutically acceptable salts, solvates, and hydrates thereof:

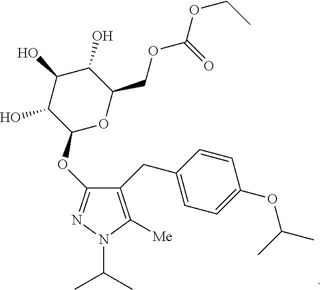 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

In some embodiments, the first pharmaceutical agent is a sulfonylurea selected from ethyl ((2R,3S,4S,5R,6S)-3,4,5-trihydroxy-6-(2-(4-methoxybenzyl)phenoxy)tetrahydro-2H-pyran-2-yl)methyl carbonate (chemical structure shown below) and pharmaceutically acceptable salts, solvates, and hydrates thereof:

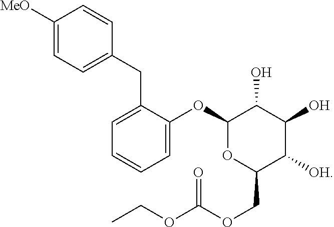 [SMILES: CCOC(=O)OC[C@H]1O[C@@H](Oc2ccccc2Cc2ccc(OC)cc2)[C@H](O)[C@@H](O)[C@@H]1O]

In some embodiments, the first pharmaceutical agent is an SGLT2 inhibitor selected from: dapagliflozin, remogliflozin, and sergliflozin. In some embodiments, the first pharmaceutical agent is dapagliflozin. In some embodiments, the first pharmaceutical agent is remogliflozin. In some embodiments, the first pharmaceutical agent is sergliflozin.

In some embodiments, the second pharmaceutical agent is a sulfonylurea selected from (2S,3R,4R,5S,6R)-2-(4-chloro-3-(4-ethoxybenzyl)phenyl)-6-(hydroxymethyl)tetrahydro-2H-pyran-3,4,5-triol (chemical structure shown below) and pharmaceutically acceptable salts, solvates, and hydrates thereof:

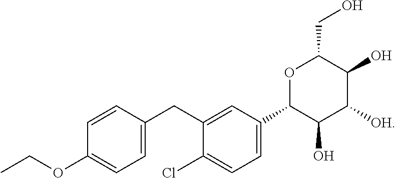 [SMILES: CCOc1ccc(Cc2cc([C@@H]3O[C@H](CO)[C@@H](O)[C@H](O)[C@H]3O)ccc2Cl)cc1]

In some embodiments, the second pharmaceutical agent is a sulfonylurea selected from ethyl ((2R,3S,4S,5R,6S)-3,4,5-trihydroxy-6-(4-(4-isopropoxybenzyl)-1-isopropyl-5-methyl-1H-pyrazol-3-yloxy)tetrahydro-2H-pyran-2-yl)methyl carbonate (chemical structure shown below) and pharmaceutically acceptable salts, solvates, and hydrates thereof:

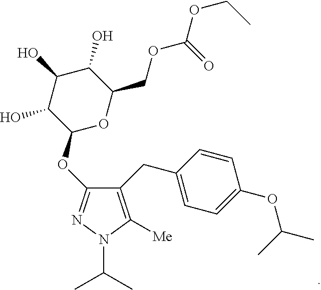 [SMILES: CCOC(=O)OC[C@H]1O[C@@H](Oc2nn(C(C)C)c(C)c2Cc2ccc(OC(C)C)cc2)[C@H](O)[C@@H](O)[C@@H]1O]

In some embodiments, the second pharmaceutical agent is a sulfonylurea selected from ethyl ((2R,3S,4S,5R,6S)-3,4,5-trihydroxy-6-(2-(4-methoxybenzyl)phenoxy)tetrahydro-2H-pyran-2-yl)methyl carbonate (chemical structure shown below) and pharmaceutically acceptable salts, solvates, and hydrates thereof:

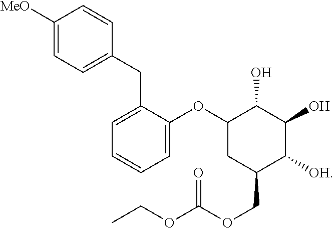 [SMILES: CCOC(=O)OC[C@H]1CC(Oc2ccccc2Cc2ccc(OC)cc2)[C@H](O)[C@@H](O)[C@@H]1O]

In some embodiments, the second pharmaceutical agent is an SGLT2 inhibitor selected from: dapagliflozin, remogliflozin, and sergliflozin. In some embodiments, the second pharmaceutical agent is dapagliflozin. In some embodiments, the second pharmaceutical agent is remogliflozin. In some embodiments, the second pharmaceutical agent is sergliflozin.

Astellas and Kotobuki disclosed a series of SGLT2 inhibitors in international patent publication WO2004/080990. Some embodiments of the present invention include every combination of one or more compounds selected from compounds disclosed in WO2004/080990 and pharmaceutically acceptable salts, solvates, and hydrates thereof.

Aventis disclosed a series of SGLT2 inhibitors in international patent publication WO2004/007517. Some embodiments of the present invention include every combination of one or more compounds selected from compounds disclosed in WO2004/007517 and pharmaceutically acceptable salts, solvates, and hydrates thereof. One such compound is (2R,3S,4S,5R,6S)-2-(hydroxymethyl)-6-(2-(4-methoxybenzyl)thiophen-3-yloxy)tetrahydro-2H-pyran-3,4,5-triol. In some embodiments, the SGLT2 inhibitor is selected from (2R,3S,4S,5R,6S)-2-(hydroxymethyl)-6-(2-(4-methoxybenzyl)thiophen-3-yloxy)tetrahydro-2H-pyran-3,4,5-triol, and pharmaceutically acceptable salts, solvates, and hydrates thereof:

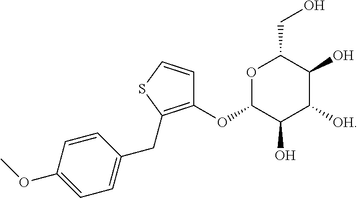 [SMILES: COc1ccc(Cc2sccc2O[C@@H]2O[C@H](CO)[C@@H](O)[C@H](O)[C@H]2O)cc1]

Tanabe disclosed a series of SGLT2 inhibitors in international patent publication WO2005/012326. Some embodiments of the present invention include every combination of one or more compounds selected from compounds disclosed in WO2005/012326 and pharmaceutically acceptable salts, solvates, and hydrates thereof. One such compound is (2S,3R,4R,5S,6R)-2-(34(5-(4-fluorophenyl)thiophen-2-yl)methyl)-4-methylphenyl)-6-(hydroxymethyl)tetrahydro-2H-pyran-3,4,5-triol. In some embodiments, the SGLT2 inhibitor is selected from (2S,3R,4R,5S,6R)-2-(3-((5-(4-fluorophenyl)thiophen-2-yl)methyl)-4-methylphenyl)-6-(hydroxymethyl)tetrahydro-2H-pyran-3,4,5-triol, and pharmaceutically acceptable salts, solvates, and hydrates thereof:

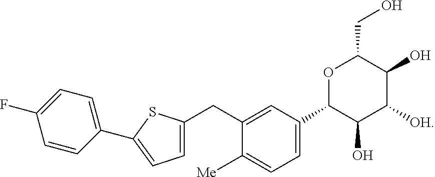 [SMILES: Cc1ccc([C@@H]2O[C@H](CO)[C@@H](O)[C@H](O)[C@H]2O)cc1Cc1ccc(-c2ccc(F)cc2)s1]

Boehringer Ingelheim disclosed a series of SGLT2 inhibitors in international patent publication WO2005/092877. Some embodiments of the present invention include every combination of one or more compounds selected from compounds disclosed in WO2005/092877 and pharmaceutically acceptable salts, solvates, and hydrates thereof.

Chugai disclosed a series of SGLT2 inhibitors in international patent publication WO2006/080421. Some embodiments of the present invention include every combination of one or more compounds selected from compounds disclosed in WO2006/080421 and pharmaceutically acceptable salts, solvates, and hydrates thereof.

Lexicon disclosed a series of SGLT2 inhibitors in international patent publication WO2008/109591. Some embodiments of the present invention include every combination of one or more compounds selected from compounds disclosed in WO2008/109591 and pharmaceutically acceptable salts, solvates, and hydrates thereof.

### Meglitinides

The meglitinides promote secretion of insulin by binding to the pancreatic beta-cells in a similar manner as sulfonylureas but at an alternative binding site. Examples of meglitinides include Novo Nordisk's repaglinide (Prandin, (S)-2-ethoxy-4-(2-(3-methyl-1-(2-(piperidin-1-yl)phenyl)butylamino)-2-oxoethyl)benzoic acid), nateglinide (Starlix, (R)-2-((1r,4R)-4-isopropylcyclohexanecarboxamido)-3-phenylpropanoic acid), mitiglinide ((S)-2-benzyl-4-((3aR,7aS)-1H-isoindol-2(3H,3aH,4H,5H,6H,7H,7aH)-yl)-4-oxobutanoic acid), and the like.

In some embodiments, the first pharmaceutical agent is (S)-2-ethoxy-4-(2-(3-methyl-1-(2-(piperidin-1-yl)phenyl)butylamino)-2-oxoethyl)benzoic acid (chemical structure shown below) and pharmaceutically acceptable salts, solvates, and hydrates thereof:

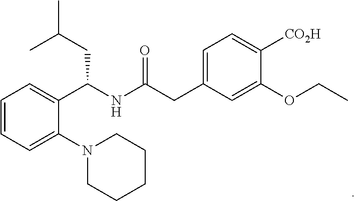 [SMILES: CCOc1cc(CC(=O)N[C@@H](CC(C)C)c2ccccc2N2CCCCC2)ccc1C(=O)O]

In some embodiments, the first pharmaceutical agent is a sulfonylurea selected from (R)-2-((1r,4R)-4-isopropylcyclohexanecarboxamido)-3-phenylpropanoic acid (chemical structure shown below) and pharmaceutically acceptable salts, solvates, and hydrates thereof:

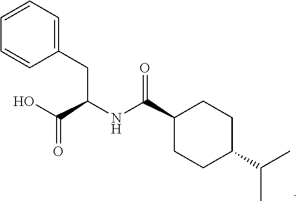 [SMILES: CC(C)[C@H]1CC[C@H](C(=O)N[C@H](Cc2ccccc2)C(=O)O)CC1]

In some embodiments, the first pharmaceutical agent is a sulfonylurea selected from (S)-2-benzyl-4-((3aR,7aS)-1H-isoindol-2(3H,3aH,4H,5H,6H,7H,7aH)-yl)-4-oxobutanoic acid (chemical structure shown below) and pharmaceutically acceptable salts, solvates, and hydrates thereof:

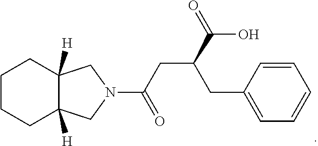 [SMILES: O=C(O)[C@H](CC(=O)N1C[C@H]2CCCC[C@H]2C1)Cc1ccccc1]

In some embodiments, the first pharmaceutical agent is a meglitinide selected from the following meglitinides: repaglinide, nateglinide, mitiglinide, and pharmaceutically acceptable salts, solvates, and hydrates thereof. In some embodiments, the first pharmaceutical agent is a meglitinide selected from repaglinide and pharmaceutically acceptable salts, solvates, and hydrates thereof. In some embodiments, the first pharmaceutical agent is a meglitinide selected from nateglinide and pharmaceutically acceptable salts, solvates, and hydrates thereof. In some embodiments, the first pharmaceutical agent is a meglitinide selected from mitiglinide and pharmaceutically acceptable salts, solvates, and hydrates thereof.

In some embodiments, the second pharmaceutical agent is (S)-2-ethoxy-4-(2-(3-methyl-1-(2-(piperidin-1-yl)phenyl)butylamino)-2-oxoethyl)benzoic acid (chemical structure shown below) and pharmaceutically acceptable salts, solvates, and hydrates thereof:

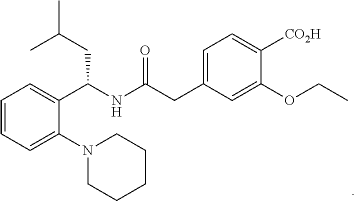 [SMILES: CCOc1cc(CC(=O)N[C@@H](CC(C)C)c2ccccc2N2CCCCC2)ccc1C(=O)O]

In some embodiments, the second pharmaceutical agent is a sulfonylurea selected from (R)-2-((1r,4R)-4-isopropylcyclohexanecarboxamido)-3-phenylpropanoic acid (chemical structure shown below) and pharmaceutically acceptable salts, solvates, and hydrates thereof:

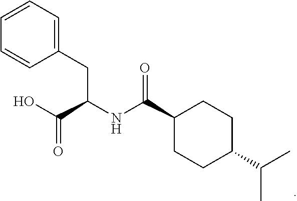 [SMILES: CC(C)[C@H]1CC[C@H](C(=O)N[C@H](Cc2ccccc2)C(=O)O)CC1]

In some embodiments, the second pharmaceutical agent is a sulfonylurea selected from (S)-2-benzyl-4-((3aR,7aS)-1H-isoindol-2(3H,3aH,4H,5H,6H,7H,7aH)-yl)-4-oxobutanoic acid (chemical structure shown below) and pharmaceutically acceptable salts, solvates, and hydrates thereof:

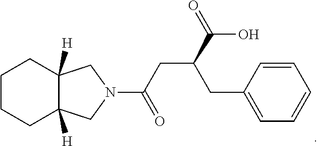 [SMILES: O=C(O)[C@H](CC(=O)N1C[C@H]2CCCC[C@H]2C1)Cc1ccccc1]

In some embodiments, the second pharmaceutical agent is a meglitinide selected from the following meglitinides: repaglinide, nateglinide, mitiglinide, and pharmaceutically acceptable salts, solvates, and hydrates thereof. In some embodiments, the second pharmaceutical agent is a meglitinide selected from repaglinide and pharmaceutically acceptable salts, solvates, and hydrates thereof. In some embodiments, the second pharmaceutical agent is a meglitinide selected from nateglinide and pharmaceutically acceptable salts, solvates, and hydrates thereof. In some embodiments, the second pharmaceutical agent is a meglitinide selected from mitiglinide and pharmaceutically acceptable salts, solvates, and hydrates thereof.

### Thiazolidinediones

Thiazolidinediones belong to the class of drugs more commonly known as TZDs. These drugs act by binding to the nuclear receptor peroxisome proliferator-activated receptor gamma (PPARγ) activate transcription of a number of specific genes leading to a decrease in insulin resistance. Examples of thiazolidinediones include rosiglitazone (Avandia, 5-(4-(2-(methyl(pyridin-2-yl)amino)ethoxy)benzyl)thiazolidine-2,4-dione), pioglitazone (Actos, 5-(4-(2-(5-ethylpyridin-2-yl)ethoxy)benzyl)thiazolidine-2,4-dione), troglitazone (Rezulin, 5-(4-((6-hydroxy-2,5,7,8-tetramethylchroman-2-yl)methoxy)benzyl)thiazolidine-2,4-dione), rivoglitazone (5-(4-((6-methoxy-1-methyl-1H-benzo[d]imidazol-2-yl)methoxy)benzyl)thiazolidine-2,4-dione), ciglitazone(5-(4-((1-methylcyclohexyl)methoxy)benzyl)thiazolidine-2,4-dione), and thiazolidinediones known in the art.

In some embodiments, the first pharmaceutical agent is 5-(4-(2-(methyl(pyridin-2-yl)amino)ethoxy)benzyl)thiazolidine-2,4-dione (chemical structure shown below) and pharmaceutically acceptable salts, solvates, and hydrates thereof:

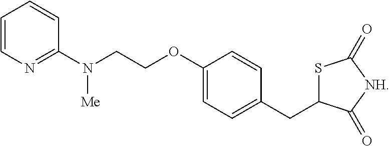 [SMILES: CN(CCOc1ccc(CC2SC(=O)NC2=O)cc1)c1ccccn1]

In some embodiments, the first pharmaceutical agent is 5-(4-(2-(5-ethylpyridin-2-yl)ethoxy)benzyl)thiazolidine-2,4-dione (chemical structure shown below) and pharmaceutically acceptable salts, solvates, and hydrates thereof:

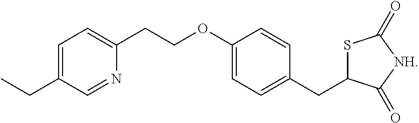 [SMILES: CCc1ccc(CCOc2ccc(CC3SC(=O)NC3=O)cc2)nc1]

In some embodiments, the first pharmaceutical agent is 5-(4-((6-hydroxy-2,5,7,8-tetramethylchroman-2-yl)methoxy)benzyl)thiazolidine-2,4-dione (chemical structure shown below) and pharmaceutically acceptable salts, solvates, and hydrates thereof:

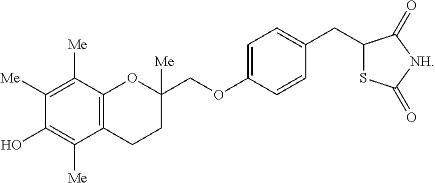 [SMILES: Cc1c(C)c2c(c(C)c1O)CCC(C)(COc1ccc(CC3SC(=O)NC3=O)cc1)O2]

In some embodiments, the first pharmaceutical agent is 5-(4-((6-methoxy-1-methyl-1H-benzo[d]imidazol-2-yl)methoxy)benzyl)thiazolidine-2,4-dione (chemical structure shown below) and pharmaceutically acceptable salts, solvates, and hydrates thereof:

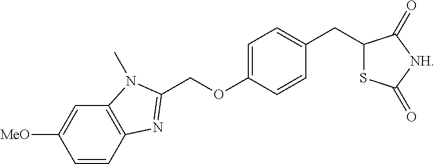 [SMILES: COc1ccc2nc(COc3ccc(CC4SC(=O)NC4=O)cc3)n(C)c2c1]

In some embodiments, the first pharmaceutical agent is 5-(4-((1-methylcyclohexyl)methoxy)benzyl)thiazolidine-2,4-dione (chemical structure shown below) and pharmaceutically acceptable salts, solvates, and hydrates thereof:

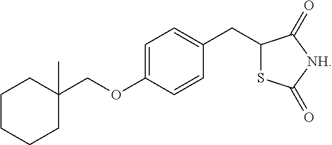 [SMILES: CC1(COc2ccc(CC3SC(=O)NC3=O)cc2)CCCCC1]

In some embodiments, the first pharmaceutical agent is a thiazolidinedione selected from rosiglitazone and pharmaceutically acceptable salts, solvates, and hydrates thereof. In some embodiments, the first pharmaceutical agent is a thiazolidinedione selected from pioglitazone and pharmaceutically acceptable salts, solvates, and hydrates thereof. In some embodiments, the first pharmaceutical agent is a thiazolidinedione selected from troglitazone and pharmaceutically acceptable salts, solvates, and hydrates thereof. In some embodiments, the first pharmaceutical agent is a thiazolidinedione selected from rivoglitazone and pharmaceutically acceptable salts, solvates, and hydrates thereof. In some embodiments, the first pharmaceutical agent is a thiazolidinedione selected from ciglitazone and pharmaceutically acceptable salts, solvates, and hydrates thereof.

In some embodiments, the second pharmaceutical agent is 5-(4-(2-(methyl(pyridin-2-yl)amino)ethoxy)benzyl)thiazolidine-2,4-dione (chemical structure shown below) and pharmaceutically acceptable salts, solvates, and hydrates thereof:

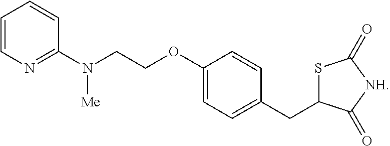 [SMILES: CN(CCOc1ccc(CC2SC(=O)NC2=O)cc1)c1ccccn1]

In some embodiments, the second pharmaceutical agent is 5-(4-(2-(5-ethylpyridin-2-yl)ethoxy)benzyl)thiazolidine-2,4-dione (chemical structure shown below) and pharmaceutically acceptable salts, solvates, and hydrates thereof:

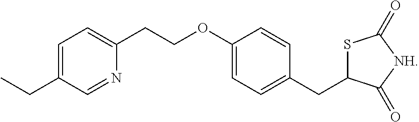 [SMILES: CCc1ccc(CCOc2ccc(CC3SC(=O)NC3=O)cc2)nc1]

In some embodiments, the second pharmaceutical agent is 5-(4-((6-hydroxy-2,5,7,8-tetramethylchroman-2-yl)methoxy)benzyl)thiazolidine-2,4-dione (chemical structure shown below) and pharmaceutically acceptable salts, solvates, and hydrates thereof:

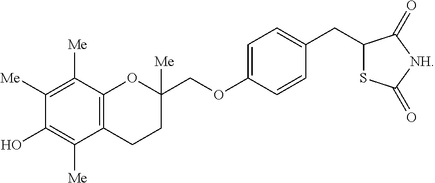 [SMILES: Cc1c(C)c2c(c(C)c1O)CCC(C)(COc1ccc(CC3SC(=O)NC3=O)cc1)O2]

In some embodiments, the second pharmaceutical agent is 5-(4-((6-methoxy-1-methyl-1H-benzo[d]imidazol-2-yl)methoxy)benzyl)thiazolidine-2,4-dione (chemical structure shown below) and pharmaceutically acceptable salts, solvates, and hydrates thereof:

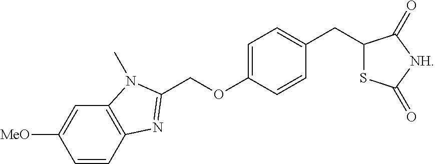 [SMILES: COc1ccc2nc(COc3ccc(CC4SC(=O)NC4=O)cc3)n(C)c2c1]

In some embodiments, the second pharmaceutical agent is 5-(4-((1-methylcyclohexyl)methoxy)benzyl)thiazolidine-2,4-dione (chemical structure shown below) and pharmaceutically acceptable salts, solvates, and hydrates thereof:

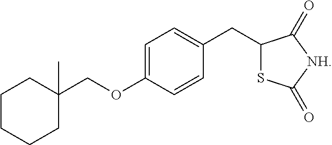 [SMILES: CC1(COc2ccc(CC3SC(=O)NC3=O)cc2)CCCCC1]

In some embodiments, the second pharmaceutical agent is a thiazolidinedione selected from rosiglitazone and pharmaceutically acceptable salts, solvates, and hydrates thereof. In some embodiments, the second pharmaceutical agent is a thiazolidinedione selected from pioglitazone and pharmaceutically acceptable salts, solvates, and hydrates thereof. In some embodiments, the second pharmaceutical agent is a thiazolidinedione selected from troglitazone and pharmaceutically acceptable salts, solvates, and hydrates thereof. In some embodiments, the second pharmaceutical agent is a thiazolidinedione selected from rivoglitazone and pharmaceutically acceptable salts, solvates, and hydrates thereof. In some embodiments, the second pharmaceutical agent is a thiazolidinedione selected from ciglitazone and pharmaceutically acceptable salts, solvates, and hydrates thereof.

### Anti-Diabetic Peptide Analogues

Anti-diabetic peptide analogues are peptides that promote secretion of insulin by acting as an incretin mimetic, such as, GLP-1 and GIP. Examples of an anti-diabetic peptide analog include, exenatide, liraglutide, taspoglutide, and anti-diabetic peptides analogues know in the art.

In some embodiments, the first pharmaceutical agent is an anti-diabetic peptide analogue selected from: exenatide; liraglutide; and taspoglutide. In some embodiments, the first pharmaceutical agent is exenatide. In some embodiments, the first pharmaceutical agent is liraglutide. In some embodiments, the first pharmaceutical agent is taspoglutide.

In some embodiments, the first pharmaceutical agent is L-histidylglycyl-L-α-glutamylglycyl-L-threonyl-L-phenylalanyl-L-threonyl-L-seryl-L-α-aspartyl-L-leucyl-L-seryl-L-lysyl-L-glutaminyl-L-methionyl-L-α-glutamyl-L-α-glutamyl-L-α-glutamyl-L-alanyl-L-valyl-L-arginyl-L-leucyl-L-phenylalanyl-L-isoleucyl-L-α-glutamyl-L-tryptophyl-L-leucyl-L-lysyl-L-asparaginylglycylglycyl-L-prolyl-L-seryl-L-serylglycyl-L-alanyl-L-prolyl-L-prolyl-L-prolyl-L-serinamide (i.e., exenatide) and pharmaceutically acceptable salts, solvates, and hydrates thereof.

In some embodiments, the first pharmaceutical agent is L-histidyl-L-alanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N-6-[N-(1-oxohexadecyl)-L-α-glutamyl]-L-lysyl-L-α-glutamyl-L-phenylalanyl-L-isoleucyl-L-alanyl-L-tryptophyl-L-leucyl-L-valyl-L-arginylglycyl-L-arginyl-glycine (liraglutide) and pharmaceutically acceptable salts, solvates, and hydrates thereof.

In some embodiments, the first pharmaceutical agent is H₂N-His-2-methyl-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-2-methyl-Ala-Arg-CONH₂ (taspoglutide) and pharmaceutically acceptable salts, solvates, and hydrates thereof.

In some embodiments, the second pharmaceutical agent is an anti-diabetic peptide analogue selected from: exenatide; liraglutide; and taspoglutide. In some embodiments, the second pharmaceutical agent is exenatide. In some embodiments, the second pharmaceutical agent is liraglutide. In some embodiments, the second pharmaceutical agent is taspoglutide.

In some embodiments, the second pharmaceutical agent is L-histidylglycyl-L-α-glutamylglycyl-L-threonyl-L-phenylalanyl-L-threonyl-L-seryl-L-α-aspartyl-L-leucyl-L-seryl-L-lysyl-L-glutaminyl-L-methionyl-L-α-glutamyl-L-α-glutamyl-L-α-glutamyl-L-alanyl-L-valyl-L-arginyl-L-leucyl-L-phenylalanyl-L-isoleucyl-L-α-glutamyl-L-tryptophyl-L-leucyl-L-lysyl-L-asparaginylglycylglycyl-L-prolyl-L-seryl-L-serylglycyl-L-alanyl-L-prolyl-L-prolyl-L-prolyl-L-serinamide (i.e., exenatide) and pharmaceutically acceptable salts, solvates, and hydrates thereof.

In some embodiments, the second pharmaceutical agent is L-histidyl-L-alanyl-L-α-glutamylglycyl-L-threonyl-L-phenylalanyl-L-threonyl-L-seryl-L-α-aspartyl-L-valyl-L-seryl-L-seryl-L-tyrosyl-L-leucyl-L-α-glutamylglycyl-L-glutaminyl-L-alanyl-L-alanyl-N-6-[N-(1-oxohexadecyl)-L-α-glutamyl]-L-lysyl-L-α-glutamyl-L-phenylalanyl-L-isoleucyl-L-alanyl-L-tryptophyl-L-leucyl-L-valyl-L-arginylglycyl-L-arginyl-glycine (liraglutide) and pharmaceutically acceptable salts, solvates, and hydrates thereof.

In some embodiments, the second pharmaceutical agent is H₂N-His-2-methyl-Ala-Glu-Gly-Thr-Phe-Thr-Ser-Asp-Val-Ser-Ser-Tyr-Leu-Glu-Gly-Gln-Ala-Ala-Lys-Glu-Phe-Ile -Ala-Trp-Leu-Val-Lys-2-methyl-Ala-Arg-CONH₂ (taspoglutide) and pharmaceutically acceptable salts, solvates, and hydrates thereof.

### Other Utilities

Another object of the present invention relates to radiolabeled compounds of the present invention that would be useful not only in radio-imaging but also in assays, both in vitro and in vivo, for localizing and quantitating GPR119 receptors in tissue samples, including human and for identifying GPR119 receptor ligands by inhibition binding of a radiolabeled compound. It is a further object of this invention to develop novel GPR119 receptor assays of which comprise such radiolabeled compounds.

The present disclosure includes all isotopes of atoms occurring in the present compounds, intermediates, salts and crystalline forms thereof. Isotopes include those atoms having the same atomic number but different mass numbers. One aspect of the present invention includes every combination of one or more atoms in the present compounds, intermediates, salts, and crystalline forms thereof that is replaced with an atom having the same atomic number but a different mass number. One such example is the replacement of an atom that is the most naturally abundant isotope, such as ¹H or ¹²C, found in one the present compounds, intermediates, salts, and crystalline forms thereof, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling of the present compounds, intermediates, salts, and crystalline forms thereof can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, intermediates, salts, and crystalline forms thereof, wherein the naturally occurring distribution of the isotopes in the composition is perturbed. Another aspect of the present invention includes compositions and pharmaceutical compositions comprising compounds as described herein wherein the compound is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or a scarcer radio-isotope or nonradioactive isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas: This procedure normally yields high specific activity products and requires halogenated or unsaturated precursors.

B. Reduction with Sodium Borohydride [³H]: This procedure is rather inexpensive and requires precursors containing reducible functional groups such as aldehydes, ketones, lactones, esters and the like.

C. Reduction with Lithium Aluminum Hydride [³H]: This procedure offers products at almost theoretical specific activities. It also requires precursors containing reducible functional groups such as aldehydes, ketones, lactones, esters and the like.

D. Tritium Gas Exposure Labeling: This procedure involves exposing precursors containing exchangeable protons to tritium gas in the presence of a suitable catalyst.

E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A representative procedure was reported by Zhu, G-D. and co-workers in J. Org. Chem., 2002, 67, 943-948.

B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled form of Compound 1 can be used in a screening assay to identify/evaluate compounds. In general terms, a newly synthesized or identified compound (i.e., test compound) can be evaluated for its ability to reduce binding of a radiolabeled form of Compound 1 to a GPR119 receptor. The ability of a test compound to compete with a radiolabeled form of Compound 1 for the binding to a GPR119 receptor directly correlates to its binding affinity.

Certain labeled compounds of the present invention bind to certain GPR119 receptors. In one embodiment the labeled compound has an IC₅₀ less than about 500 μM. In one embodiment the labeled compound has an IC₅₀ less than about 100 μM. In one embodiment the labeled compound has an IC₅₀ less than about 10 μM. In one embodiment the labeled compound has an IC₅₀ less than about 1 μM. In one embodiment the labeled compound has an IC₅₀ less than about 0.1 μM. In one embodiment the labeled compound has an IC₅₀ less than about 0.01 μM. In one embodiment the labeled compound has an IC₅₀ less than about 0.005 μM.

Other uses of the disclosed receptors and methods will become apparent to those skilled in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

### Example 1

### Syntheses of Compounds of the Present Invention

The compounds of the invention and their syntheses are further illustrated by the following examples. The following examples are provided to further define the invention without, however, limiting the invention to the particulars of these examples. The compounds described herein, supra and infra, are named according to AutoNom version 2.2, AutoNom 2000, CS ChemDraw Ultra Version 7.0.1, or CS ChemDraw Ultra Version 9.0.7. In certain instances literature names and/or common names are used and it is understood that these names would be recognized by those skilled in the art.

Proton nuclear magnetic resonance (¹H NMR) spectra were recorded on a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublets, ddd=doublet of doublet of doublets, dt=doublet of triplets, t=triplet, td=triplet of doublets, tt=triplet of triplets, q=quartet, m=multiplet, bs=broad singlet, bt=broad triplet. Microwave irradiations were carried out using a Smith Synthesizer™ or an Emrys Optimizer™ (Biotage). Thin-layer chromatography (TLC) was performed on silica gel 60 F₂₅₄ (Merck), preparatory thin-layer chromatography (prep TLC) was preformed on PK6F silica gel 60 A 1 mm plates (Whatman) and column chromatography was carried out on a silica gel column using Kieselgel 60, 0.063-0.200 mm (Merck). Evaporation was done under reduced pressure on a Büchi rotary evaporator.

LCMS spec: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of 1-Methylcyclopropyl 4-(5-Fluoro-6-(2-methyl-6-(methylsulfonyl)pyridin-3-yloxy)pyrimidin-4-yloxy)piperidine-1-carboxylate (Compound 1)

### Step A: Preparation of tert-Butyl 4-(6-Chloro-5-fluoropyrimidin-4-yloxy)piperidine-1-carboxylate

To a solution of 4,6-dichloro-5-fluoropyrimidine (1.00 g, 5.99 mmol) and tert-butyl 4-hydroxypiperidine-1-carboxylate (1.205 g, 5.99 mmol) in THF (10 mL) at −78° C. was added 1 M potassium tert-butoxide solution in THF (5.99 mL, 5.99 mmol) dropwise. The mixture became thick and additional THF (10 mL) was slowly added. After stiffing for 15 min, the mixture was diluted with water and extracted with EtOAc. The organic layer was concentrated under reduced pressure and purified by silica gel flash column chromatography to give the title compound as a colorless oil, which solidified slowly (1.9561 g, 98%). Exact mass calculated for C₁₄H₁₉ClFN₃O₃: 331.1. found: LCMS m/z=332.4 [M+H]⁺; ¹H NMR (400 MHz, CDCl₃) δ ppm 1.45 (s, 9H), 1.75-1.85 (m, 2H), 1.98-2.04 (m, 2H), 3.28-3.36 (m, 2H), 3.75-3.81 (m, 2H), 5.30-5.39 (m, 1H), 8.31 (s, 1H).

### Step B: Preparation of tert-Butyl 4-(5-Fluoro-6-(2-methyl-6-(methylsulfonyl)pyridin-3-yloxy)pyrimidin-4-yloxy)piperidine-1-carboxylate

A suspension of tert-butyl 4-(6-chloro-5-fluoropyrimidin-4-yloxy)piperidine-1-carboxylate (1.47 g, 4.43 mmol), 2-methyl-6-(methylsulfonyl)pyridin-3-ol (0.912 g, 4.87 mmol), and potassium carbonate (1.225 g, 8.86 mmol) in DMF (30.0 mL) was heated at 100° C. for 1 h. The mixture was filtered and the filtrate was concentrated under reduced pressure. The residue was purified by silica gel flash column chromatography to give the title compound as an off-white solid (1.52 g, 71.1%). Exact mass calculated for C₂₁H₂₇FN₄O₆S: 482.2. found: LCMS m/z=483.4 [M+H]⁺; ¹H NMR (400 MHz, CDCl₃) δ ppm 1.48 (s, 9H), 1.78-1.88 (m, 2H), 1.99-2.07 (m, 2H), 2.55 (s, 3H), 3.25 (s, 3H), 3.29-3.36 (m, 2H), 3.77-3.85 (m, 2H), 5.35-5.42 (m, 1H), 7.67 (d, J=8.4 Hz, 1H), 8.01 (d, J=8.4 Hz, 1H), 8.07 (s, 1H).

### Step C: Preparation of 5-Fluoro-4-(2-methyl-6-(methylsulfonyl)pyridin-3-yloxy)-6-(piperidin-4-yloxy)pyrimidine Hydrochloride

To a solution of tert-butyl 4-(5-fluoro-6-(2-methyl-6-(methylsulfonyl)pyridin-3-yloxy)pyrimidin-4-yloxy)piperidine-1-carboxylate (1.52 g, 3.15 mmol) in DCM (5 mL) was added 4 N hydrogen chloride solution in dioxane (4.43 mL, 17.72 mmol). The mixture was stirred at room temperature for 1 h. Additional 4 N hydrogen chloride solution in dioxane (4.43 mL, 17.72 mmol) was added and the mixture stirred at room temperature for an additional 1 h. The mixture was concentrated under reduced pressure and the residue was dried under reduced pressure at 50° C. to give the title compound as a light-yellow solid (1.32 g, 100%). Exact mass calculated for C₁₆H₁₉FN₄O₄S: 382.2. found: LCMS m/z=383.4 [M+H]⁺; ¹H NMR (400 MHz, CDCl₃) δ ppm 2.25-2.35 (m, 2H), 2.38-2.48 (m, 2H), 2.55 (s, 3H), 3.26 (s, 3H), 3.33-3.48 (m, 4H), 5.52-5.57 (m, 1H), 7.68 (d, J=8.4 Hz, 1H), 8.03 (d, J=8.4 Hz, 1H), 8.09 (s, 1H), 9.85 (bs, 2H).

### Step D: Preparation of 2,5-Dioxopyrrolidin-1-yl 1-methylcyclopropyl Carbonate

To a solution of 1-methylcyclopropanol (2.00 g, 27.7 mmol) in MeCN (100 mL) was added triethylamine (11.60 mL, 83 mmol) and N,N′-disuccinimidyl carbonate (10.66 g, 41.6 mmol). The mixture was stirred at room temperature over the weekend. The mixture was washed with saturated aqueous NaHCO₃ solution and brine, dried over anhydrous Na₂SO₄, and concentrated under reduced pressure. The residue was triturated with water to give the title compound as an off-white solid (3.1554 g, 53.4% yield). ¹H NMR (400 MHz, CDCl₃) δ ppm 0.73 (t, J=6.9 Hz, 2H), 1.10 (t, J=6.9 Hz, 2H), 1.63 (s, 3H), 2.82-2.84 (m, 4H).

### Step E: Preparation of 1-Methylcyclopropyl 4-(5-Fluoro-6-(2-methyl-6-(methylsulfonyl)pyridin-3-yloxy)pyrimidin-4-yloxy)piperidine-1-carboxylate (Compound 1)

To a solution of 5-fluoro-4-(2-methyl-6-(methylsulfonyl)pyridin-3-yloxy)-6-(piperidin-4-yloxy)pyrimidine hydrochloride (200 mg, 0.477 mmol) and 2,5-dioxopyrrolidin-1-yl 1-methylcyclopropyl carbonate (247 mg, 1.16 mmol) in DCM (3.5 mL) was added triethylamine (0.333 mL, 2.387 mmol). The reaction mixture was stirred at room temperature for 2 h. The mixture was concentrated under reduced pressure and the residue was purified by silica gel flash column chromatography to give the title compound as a white solid (183 mg, 80% yield). Exact mass calculated for C₂₁H₂₅FN₄O₆S: 480.15. found: LCMS m/z=481.2 [M+H]⁺; ¹H NMR (400 MHz, CDCl₃) δ ppm 0.63-0.66 (m, 2H), 0.87-0.90 (m, 2H), 1.56 (s, 3H), 1.82-1.84 (m, 2H), 2.02-2.04 (m, 2H), 2.55 (s, 3H), 3.35 (s, 3H), 3.28-3.37 (m, 2H), 3.78-3.79 (m, 2H), 5.36-5.40 (m, 1H), 7.68 (d, J=12 Hz, 1H), 8.01 (d, J=8 Hz, 1H), 8.07 (s, 1H). Solubility: <0.001 mg/mL (H₂O); 2.758 mg/mL (20% HPCD).

### Example 2

### Effects of Compound 1 on Glucose Homeostasis in Male 129SVE Mice (oGTT)

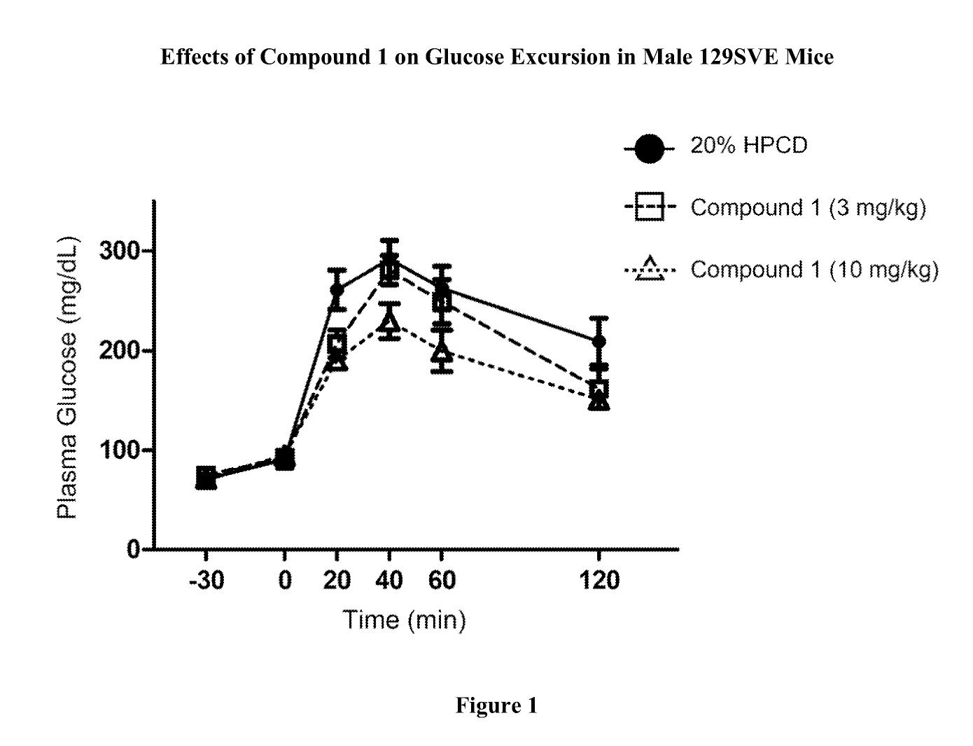

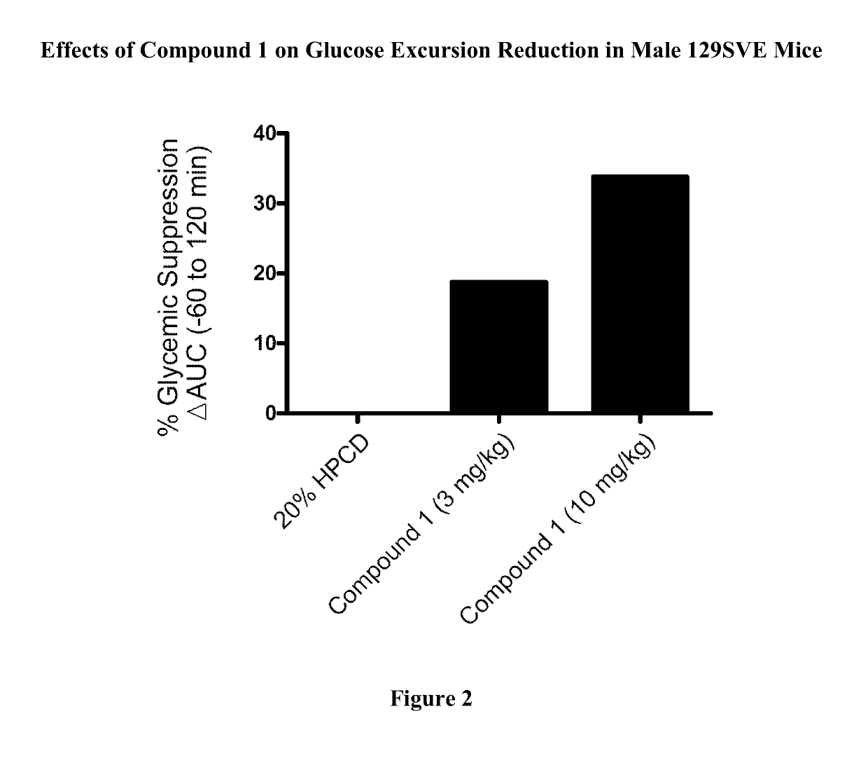

Male 129SVE mice (approximately 8 weeks old) were fasted for 18 h and randomly grouped (n=6) to receive a GPR119 agonist (Compound 1) at 3, or 10 mg/kg body weight. Compound 1 was delivered orally via a gavage needle (p.o., volume at 4 mL/kg) 30 minutes prior to glucose bolus (3 g/kg) (time=−30 min in FIG. 1), with a separate group receiving vehicle (20% hydroxypropyl-beta-cyclodextrin (HPCD) as a control. At time 0 min the glucose bolus was administered. Levels of blood glucose were assessed using a glucometer (One-Touch Ultra™, LifeScan) at time −30 min (prior to compound administration), at 0 min (when the glucose bolus was given), and at 20, 40, 60, 120 min post glucose bolus. The plasma glucose level (Table A) and glucose excursion curve (FIG. 1) are shown. Glucose excursion reduction (area under the curve (AUC)) in compound-treated animals relative to vehicle control is shown in FIG. 2. These results demonstrated that the GPR119 agonist, Compound 1, lowered blood glucose in 129SVE mice after challenge with glucose.

TABLE A

| | Plasma Glucose (mg/dL) | | | | | | | | |
|---|---|---|---|---|---|---|---|---|---|
| Time Relative | | | | Compound 1 | | | | | |
| to Glucose | 20% HPCD | | | (3 mg/kg) | | | (10 mg/kg) | | |
| Bolus (min) | Mean | SEM | n | Mean | SEM | n | Mean | SEM | n |
| −30.0 | 70.66666 | 4.45471 | 6 | 75.16666 | 3.070469 | 6 | 71.5 | 1.821172 | 6 |
| 0.0 | 90.83334 | 8.772748 | 6 | 92.0 | 2.977695 | 6 | 94.5 | 7.486655 | 6 |
| 20.0 | 261.0 | 19.75348 | 6 | 207.0 | 14.00714 | 6 | 190.3333 | 6.280481 | 6 |
| 40.0 | 291.8333 | 18.82094 | 6 | 280.6667 | 14.57547 | 6 | 229.6667 | 17.44834 | 6 |
| 60.0 | 262.6667 | 21.72352 | 6 | 248.8333 | 22.14259 | 6 | 199.6667 | 20.86411 | 6 |
| 120.0 | 209.1667 | 23.16235 | 6 | 161.8333 | 19.82493 | 6 | 150.6667 | 8.511431 | 6 |

### Example 3

### Effects of Compound 1 on GIP Release in Male 129SVE Mice

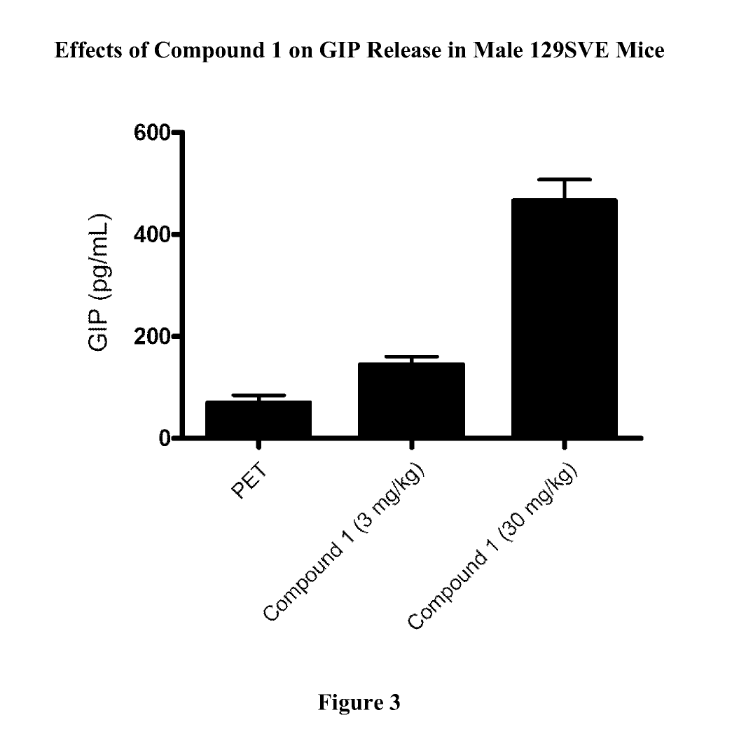

Male 129SVE mice (approximately 8 weeks old) were fasted for 18 h and randomly grouped (n=6) to receive a GPR119 agonist (Compound 1) at 3 or 30 mg/kg body weight. Compound 1 was delivered orally via a gavage needle (4 mL/kg), and after 45 min, a blood sample was collected to determine plasma total GIP levels. A separate control group received vehicle (PET (80% PEG:10% Ethanol:10% Tween80™)). Plasma GIP levels were determined using a GIP (total) ELISA kit from Millipore. The results are given in FIG. 3. These data demonstrated that the GPR119 agonist, Compound 1, stimulates the release of GIP.

### Example 4

### Homogeneous Time-Resolved Fluorescence (HTRF®) Assay for Direct cAMP Measurement

GPR119 agonist, Compound 1, was evaluated in an HTRF® cAMP detection assay (Cisbio, cAMP Dynamic 2 Assay Kit; #62AM4PEJ) according to the manufacturer's instructions using CHO-K1 cells stably expressing the GPR119 receptor. CHO-K1 cells were transduced with a lentiviral vector encoding the nucleotide sequence of GPR119 (NCBI mRNA and protein reference sequences: NM_{—}178471.2 & NP_{—}848566). The N-terminus of the GPR119 nucleotide sequence was modified to replace the first, methionine-coding, codon with a nucleotide sequence coding for a standard, nine amino acid, hemagglutinin tag. Following transduction, cells expressing the GPR119 receptor were isolated and a single clone was isolated following standard dilution-cloning procedures. On the day of the assay, cultured CHO-GPR119 cells were harvested, suspended in assay buffer and plated into 384-well assay plates (PerkinElmer® Proxiplate® #6008280) at a density of 2,000 cells per well. A cAMP standard curve was added to each plate. Compound 1 was dissolved in DMSO, serially diluted in DMSO and then diluted in assay buffer before addition to the cells. Compound 1 was evaluated in triplicate, using 10-point, 5-fold serial dilutions starting at 10 μM. The final DMSO concentration in the assay was 0.5%. Compound 1 and cells were incubated for 1 h at room temperature and then detection reagents were added to each well (cAMP-D2 in cell lysis buffer, followed by europium cryptate-labeled anti-cAMP antibody). Plates were then incubated at room temperature for 1 h prior to reading. Time-resolved fluorescence measurements were collected on PerkinElmer Envision™ or BMG Pherastar™ microplate readers. The compound N-(2-fluoro-4-(methylsulfonyl) phenyl)-6-(4-(3-isopropyl-1,2,4-oxadiazol-5-yl)piperidin-1-yl)-5-nitropyrimidin-4-amine was used as a positive control in each runset while assay buffer containing 0.5% DMSO was used as the negative control. Using the HTRF® assay, the EC₅₀ value for Compound 1 at GPR119 was observed to be 14.9 nM.

### Example 5

### Effects of Compound 1 on Glucose Homeostasis in ZDF Rats (oGTT)

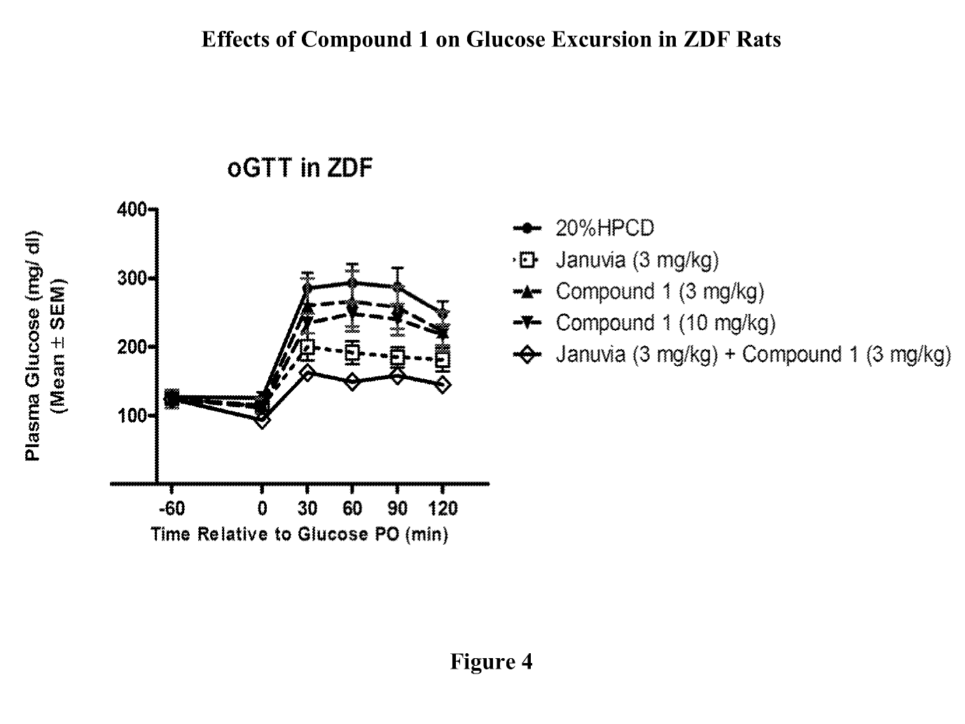

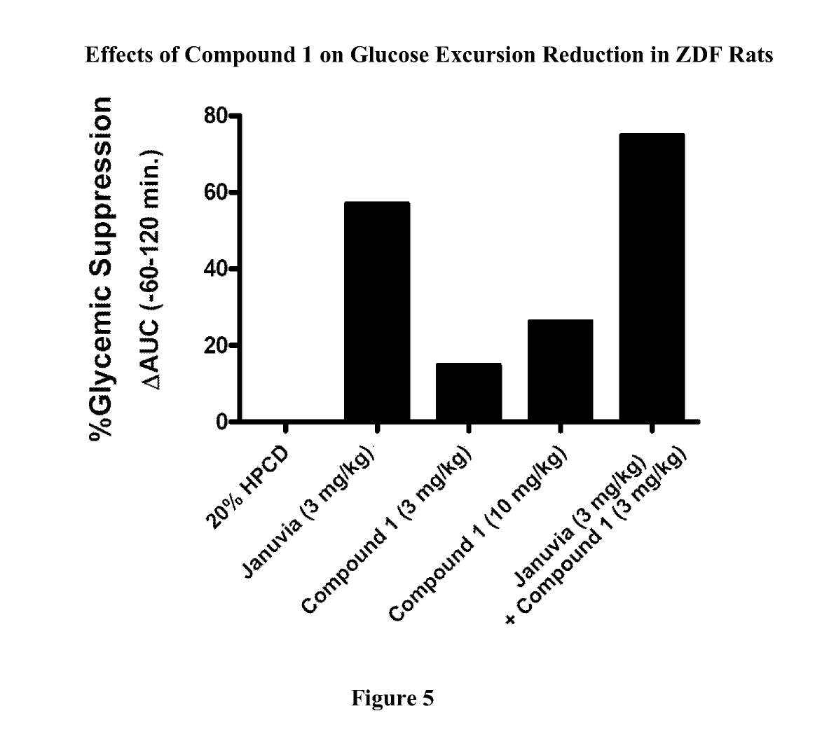

Male ZDF rats were fasted for 18 h and randomly grouped (n=6) to receive the GPR119 agonist Compound 1(3 or 10 mg/kg), the control compound, Januvia (3 mg/kg), or a combination of Compound 1(3 mg/kg) and Januvia (3 mg/kg). The compounds were delivered orally via a gavage needle (p.o., volume 4 mL/kg) 60 min prior to glucose bolus (3 g/kg) (time=−60 min in FIG. 4), with a separate group receiving vehicle (20% hydroxypropyl-beta-cyclodextrin (HPCD)) as control. At time 0 min, a glucose bolus was administered. Levels of blood glucose were assessed using a glucometer (One-Touch Ultra™, LifeScan) at time −60 min (prior to compound administration), at 0 min (at time when glucose bolus was given), and at 30, 60, 90, and 120 min post glucose bolus. The plasma glucose excursion curve (FIG. 4) is shown. Reduction in glucose excursion AUC in compound treated animals relative to vehicle control is given in FIG. 5, and in Table B. These results demonstrated that the GPR119 agonist Compound 1 lowered blood glucose after a challenge with glucose in diabetic ZDF rats.

TABLE B

| | % Inhibition of |
|---|---|
| Compound (Dose) | Glucose Excursion |
| Compound 1 (3 mg/kg) | 14.8 |
| Compound 1 (10 mg/kg) | 26.2 |
| Januvia (3 mg/kg) | 57.0 |
| Compound 1 (3 mg/kg) + Januvia (3 mg/kg) | 74.9 |

### Example 6

### Effect of Compound 1 on Gastric Emptying in SD Rats

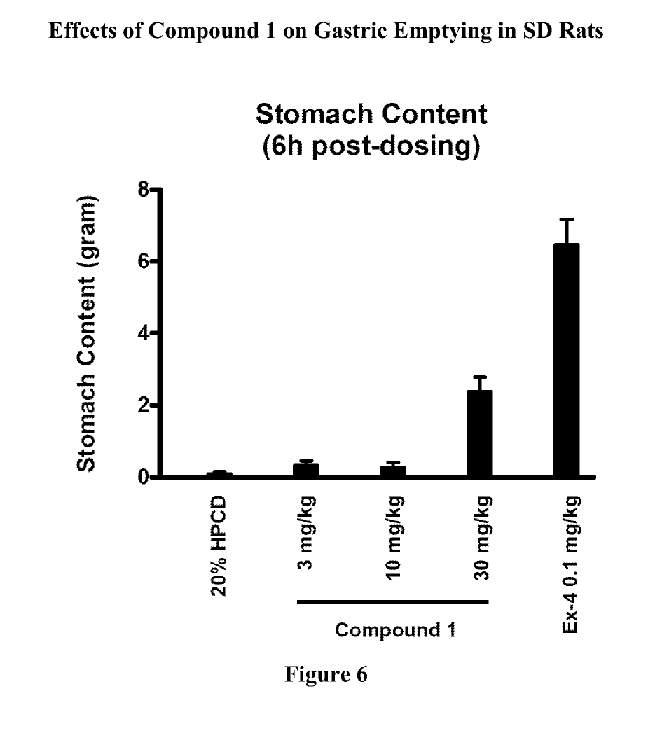

Male SD rats were fasted for 18 h and randomly grouped (n=5). Fasted rats were allowed to access food for 30 min, and the amount of food consumed was recorded for each rat. At the end of the 30 min food intake, rats were put in cages without food and water, and dosed with Compound 1 at 3, 10, or 30 mg/kg, or vehicle control (20% HPCD) orally, or with exendin-4 (Ex-4) at 100 μg/kg i.p. Six hours post-dosing, rats were euthanized with CO₂, and stomach content was determined. Effect of Compound 1 on gastric emptying was evaluated, based on the content in stomach after 6 h post-doing, compared to the positive control, Ex-4, which is known to suppress gastric emptying in rats. The results are given in FIG. 6. These results indicated that Compound 1 had an effect on gastric emptying at high dosage (30 mg/kg), but that effect was less pronounced than the positive control, Ex-4.

Those skilled in the art will recognize that various modifications, additions, substitutions, and variations to the illustrative examples set forth herein can be made without departing from the spirit of the invention and are, therefore, considered within the scope of the invention.

1. A compound selected from 1-methylcyclopropyl 4-(5-fluoro-6-(2-methyl-6-(methylsulfonyl)pyridin-3-yloxy)pyrimidin-4-yloxy)piperidine-1-carboxylate (Compound 1): 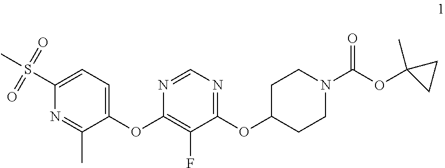 [SMILES: Cc1nc(S(C)(=O)=O)ccc1Oc1ncnc(OC2CCN(C(=O)OC3(C)CC3)CC2)c1F]
and pharmaceutically acceptable salts, solvates, and hydrates thereof.

2. A composition comprising a compound according to claim 1.

3. A composition comprising a compound according to claim 1 and a pharmaceutically acceptable carrier.

4. A method for preparing a composition comprising the step of admixing a compound according to claim 1 and a pharmaceutically acceptable carrier.

5. A composition comprising a compound according to claim 1 and a second pharmaceutical agent.

6. A method for preparing a composition comprising the step of admixing a compound according to claim 1 and a second pharmaceutical agent.

7. A pharmaceutical product selected from: a pharmaceutical composition, a formulation, a dosage form, a combined preparation, a twin pack, and a kit; comprising a compound according to claim 1 and a second pharmaceutical agent.

8. A method for increasing the secretion of an incretin in an individual, or for increasing a blood incretin level in an individual, comprising administering to said individual in need thereof, a therapeutically effective amount of: a compound according to claim 1.

9. A method for the treatment of a disorder selected from: a GPR119-receptor-related disorder; a condition ameliorated by increasing a blood incretin level; a condition ameliorated by increasing the secretion of an incretin; a condition characterized by low bone mass; a neurological disorder; a metabolic-related disorder; and obesity; in an individual; comprising administering to said individual in need thereof, a therapeutically effective amount of: a compound according to claim 1.

10-19. (canceled)

20. The method according to claim 9; wherein said incretin is GLP-1.

21. The method according to claim 9; wherein said incretin is GIP.

22. The method according to claim 9; wherein said incretin is PYY.

23. The method according to claim 9; wherein said disorder is a condition characterized by low bone mass selected from: osteopenia, osteoporosis, rheumatoid arthritis, osteoarthritis, periodontal disease, alveolar bone loss, osteotomy bone loss, childhood idiopathic bone loss, Paget's disease, bone loss due to metastatic cancer, osteolytic lesions, curvature of the spine, and loss of height.

24. The method according to claim 9; wherein said disorder is a neurological disorder selected from: stroke and Parkinsonism.

25. The method according to claim 9; wherein said disorder is a metabolic-related disorder selected from: diabetes, type 1 diabetes, type 2 diabetes, inadequate glucose tolerance, impaired glucose tolerance, insulin resistance, hyperglycemia, hyperlipidemia, hypertriglyceridemia, hypercholesterolemia, dyslipidemia, atherosclerosis, stroke, syndrome X, hypertension, pancreatic beta-cell insufficiency, enteroendocrine cell insufficiency, glucosuria, metabolic acidosis, cataracts, diabetic nephropathy, diabetic neuropathy, peripheral neuropathy, diabetic coronary artery disease, diabetic cerebrovascular disease, diabetic peripheral vascular disease, diabetic retinopathy, metabolic syndrome, a condition related to diabetes, myocardial infarction, learning impairment, memory impairment, a neurodegenerative disorder, a condition ameliorated by increasing a blood GLP-1 level in an individual with a neurodegenerative disorder, excitotoxic brain damage caused by severe epileptic seizures, Alzheimer's disease, Parkinson's disease, Huntington's disease, prion-associated disease, stroke, motor-neuron disease, traumatic brain injury, spinal cord injury, and obesity.

26. The method according to claim 9; wherein said disorder is type 2 diabetes.

27. The method according to claim 9, wherein said compound is administered in combination with a therapeutically effective amount of a second pharmaceutical agent, wherein said second pharmaceutical agent is selected from: an inhibitor of DPP-IV, a biguanide, an alpha-glucosidase inhibitor, an insulin analogue, a sulfonylurea, a SGLT2 inhibitor, a meglitinide, a thiazolidinedione, and an anti-diabetic peptide analogue.

28. The method according to claim 9, wherein said compound is administered in combination with a therapeutically effective amount of a second pharmaceutical agent, wherein said second pharmaceutical agent is an inhibitor of DPP-IV selected from the following inhibitors of DPP-IV and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one;
1-[2-(3-hydroxyadamant-1-ylamino)acetyl]pyrrolidine-2(S)-carbonitrile;
(1S,3S,5S)-2-[2(S)-amino-2-(3-hydroxyadamantan-1-yl)acetyl]-2-azabicyclo[3.1.0]hexane-3-carbonitrile;
2-[6-[3(R)-aminopiperidin-1-yl]-3-methyl-2,4-dioxo-1,2,3,4-tetrahydropyrimidin-1-ylmethyl]benzonitrile;
8-[3(R)-aminopiperidin-1-yl]-7-(2-butynyl)-3-methyl-1-(4-methylquinazolin-2-ylmethyl)xanthine;
1-[N-[3(R)-pyrrolidinyl]glycyl]pyrrolidin-2(R)-yl boronic acid;
4(S)-fluoro-1-[2-[(1R,3 S)-3-(1H-1,2,4-triazol-1-ylmethyl)cyclopentylamino]acetyl]pyrrolidine-2(S)-carbonitrile;
1-[(2S,3S,11bS)-2-amino-9,10-dimethoxy-2,3,4,6,7,11b-hexahydro-1H-pyrido[2,1-a]isoquinolin-3-yl]-4(S)-(fluoromethyl)pyrrolidin-2-one;
(2S,4S)-2-cyano-4-fluoro-1-[(2-hydroxy-1,1-dimethyl)ethylamino]acetylpyrrolidine;
8-(cis-hexahydro-pyrrolo[3,2-b]pyrrol-1-yl)-3-methyl-7-(3-methyl-but-2-enyl)-1-(2-oxo-2-phenylethyl)-3,7-dihydro-purine-2,6-dione;
1-((3S,4S)-4-amino-1-(4-(3,3-difluoropyrrolidin-1-yl)-1,3,5-triazin-2-yl)pyrrolidin-3-yl)-5,5difluoropiperidin-2-one;
(R)-2-((6-(3-aminopiperidin-1-yl)-3-methyl-2,4-dioxo-3,4-dihydropyrimidin-1(2H)-yl)methyl)-4-fluorobenzonitrile;
5-{(S)-2-[2-((S)-2-cyano-pyrrolidin-1-yl)-2-oxo-ethylamino]-propyl}-5-(1H-tetrazol-5-yl)10,11-dihydro-5H-dibenzo[a,d]cycloheptene-2,8-dicarboxylic acid bis-dimethylamide;
((2S,4S)-4-(4-(3-methyl-1-phenyl-1H-pyrazol-5-yl)piperazin-1-yl)pyrrolidin-2-yl)(thiazolidin-3-yl)methanone;
((2S,4S)-1-[2-[(4-ethoxycarbonylbicyclo[2.2.2]oct-1-yl)amino]acetyl]-4-fluoropyrrolidine-2-carbonitrile;
6-[(3R)-3-amino-piperidin-1-yl]-5-(2-chloro-5-fluoro-benzyl)-1,3-dimethyl-1,5dihydro-pyrrolo[3,2-d]pyrimidine-2,4-dione;
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile;
(2S)-1-{[2-(5-methyl-2-phenyl-oxazol-4-yl)-ethylamino]-acetyl}-pyrrolidine-2-carbonitrile;
(2S)-1-{[1,1-dimethyl-3-(4-pyridin-3-yl-imidazol-1-yl)-propylamino]-acetyl}-pyrrolidine-2-carbonitrile;
(3,3-difluoropyrrolidin-1-yl)-((2S,4S)-4-(4-(pyrimidin-2-yl)piperazin-1-yl)pyrrolidin-2-yl)methanone;
(2S,4S)-1-[(2S)-2-amino-3,3-bis(4-fluorophenyl)propanoyl]-4-fluoropyrrolidine-2-carbonitrile;
(2S,5R)-5-ethynyl-1-{N-(4-methyl-1-(4-carboxy-pyridin-2-yl)piperidin-4-yl)glycyl}pyrrolidine-2-carbonitrile; and
(1S,6R)-3-{[3-(trifluoromethyl)-5,6-dihydro[1,2,4]triazolo[4,3-a]pyrazin-7(8H)-yl]carbonyl}-6-(2,4,5-trifluorophenyl)cyclohex-3-en-1-amine.

29. The method according to claim 9, wherein said compound is administered in combination with a therapeutically effective amount of a second pharmaceutical agent, wherein said second pharmaceutical agent is an inhibitor of DPP-IV selected from the following inhibitor of DPP-IV and pharmaceutically acceptable salts, solvates, and hydrates thereof:
3(R)-amino-1-[3-(trifluoromethyl)-5,6,7,8-tetrahydro[1,2,4]triazolo[4,3-a]pyrazin-7-yl]-4-(2,4,5-trifluorophenyl)butan-1-one.
